SEQUENCE LISTING (4 sequences)

SEQ ID NO: 1 - protein, 1012 aa, Homo sapiens
MPASAPPRRPRPPPPSLSLLLVLLGLGGRRLRAEPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPCDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPETLERFCPEEFTVCTECSFFHTRKSLLAFIAFLFFASIISLSLIFYYSKKGRRSYK

SEQ ID NO: 2 - DNA, 5012 bp, Homo sapiens
cgcgcagcatgcccgccagcgccccgccgcgccgcccgcggccgccgccgccgtcgctgtcgctgctgctggtgctgctgggcctgggcggccgccgcctgcgtgcggagccgggcgacggcgcgcagacctgggcccgtttctcgcggcctcctgcccccgaggccgcgggcctcttccagggcaccttccccgacggcttcctctgggccgtgggcagcgccgcctaccagaccgagggcggctggcagcagcacggcaagggtgcgtccatctgggatacgttcacccaccaccccctggcacccccgggagactcccggaacgccagtctgccgttgggcgccccgtcgccgctgcagcccgccaccggggacgtagccagcgacagctacaacaacgtcttccgcgacacggaggcgctgcgcgagctcggggtcactcactaccgcttctccatctcgtgggcgcgagtgctccccaatggcagcgcgggcgtccccaaccgcgaggggctgcgctactaccggcgcctgctggagcggctgcgggagctgggcgtgcagcccgtggtcaccctgtaccactgggacctgccccagcgcctgcaggacgcctacggcggctgggccaaccgcgccctggccgaccacttcagggattacgcggagctctgcttccgccacttcggcggtcaggtcaagtactggatcaccatcgacaacccctacgtggtggcctggcacggctacgccaccgggcgcctggcccccggcatccggggcagcccgcggctcgggtacctggtggcgcacaacctcctcctggctcatgccaaagtctggcatctctacaatacttctttccgtcccactcagggaggtcaggtgtccattgccctaagctctcactggatcaatcctcgaagaatgaccgaccacagcatcaaagaatgtcaaaaatctctggactttgtactaggttggtttgccaaacccgtatttattgatggtgactatcccgagagcatgaagaataacctttcatctattctgcctgattttactgaatctgagaaaaagttcatcaaaggaactgctgacttttttgctctttgctttggacccaccttgagttttcaacttttggaccctcacatgaagttccgccaattggaatctcccaacctgaggcaactgctttcctggattgaccttgaatttaaccatcctcaaatatttattgtggaaaatggctggtttgtctcagggaccaccaagagagatgatgccaaatatatgtattacctcaaaaagttcatcatggaaaccttaaaagccatcaagctggatggggtggatgtcatcgggtataccgcatggtccctcatggatggtttcgagtggcacagaggttacagcatcaggcgtggactcttctatgttgactttctaagccaggacaagatgttgttgccaaagtcttcagccttgttctaccaaaagctgatagagaaaaatggcttccctcctttacctgaaaatcagcccctagaagggacatttccctgtgactttgcttggggagttgttgacaactacattcaagtagataccactctgtctcagtttaccgacctgaatgtttacctgtgggatgtccaccacagtaaaaggcttattaaagtggatggggttgtgaccaagaagaggaaatcctactgtgttgactttgctgccatccagccccagatcgctttactccaggaaatgcacgttacacattttcgcttctccctggactgggccctgattctccctctgggtaaccagtcccaggtgaaccacaccatcctgcagtactatcgctgcatggccagcgagcttgtccgtgtcaacatcaccccagtggtggccctgtggcagcctatggccccgaaccaaggactgccgcgcctcctggccaggcagggcgcctgggagaacccctacactgccctggcctttgcagagtatgcccgactgtgctttcaagagctcggccatcacgtcaagctttggataacgatgaatgagccgtatacaaggaatatgacatacagtgctggccacaaccttctgaaggcccatgccctggcttggcatgtgtacaatgaaaagtttaggcatgctcagaatgggaaaatatccatagccttgcaggctgattggatagaacctgcctgccctttctcccaaaaggacaaagaggtggctgagagagttttggaatttgacattggctggctggctgagcccattttcggctctggagattatccatgggtgatgagggactggctgaaccaaagaaacaattttcttcttccttatttcactgaagatgaaaaaaagctaatccagggtacctttgactttttggctttaagccattataccaccatccttgtagactcagaaaaagaagatccaataaaatacaatgattacctagaagtgcaagaaatgaccgacatcacgtggctcaactcccccagtcaggtggcggtagtgccctgggggttgcgcaaagtgctgaactggctgaagttcaagtacggagacctccccatgtacataatatccaatggaatcgatgacgggctgcatgctgaggacgaccagctgagggtgtattatatgcagaattacataaacgaagctctcaaagcccacatactggatggtatcaatctttgcggatactttgcttattcgtttaacgaccgcacagctccgaggtttggcctctatcgttatgctgcagatcagtttgagcccaaggcatccatgaaacattacaggaaaattattgacagcaatggtttcccgggcccagaaactctggaaagattttgtccagaagaattcaccgtgtgtactgagtgcagtttttttcacacccgaaagtctttactggctttcatagcttttctattttttgcttctattatttctctctcccttatattttactactcgaagaaaggcagaagaagttacaaatagttctgaacatttttctattcattcattttgaaataattatgcagacacatcagctgttaaccatttgcacctctaagtgttgtgaaactgtaaatttcatacatttgacttctagaaaacatttttgtggcttatgacagaggttttgaaatgggcataggtgatcgtaaaatattgaataatgcgaatagtgcctgaatttgttctctttttgggtgattaaaaaactgacaggcactataatttctgtaacacactaacaaaagcatgaaaaataggaaccacaccaatgcaacatttgtgcagaaatttgaatgacaagattaggaatattttcttctgcacccacttctaaatttaatgtttttctggaagtagtaattgcaagagttcgaatagaaagttatgtaccaagtaaccatttctcagctgccataataatgcctagtggcttcccctctgtcaaatctagtttcctatggaaaagaagatggcagatacaggagagacgacagagggtcctaggctggaatgttcctttcgaaagcaatgcttctatcaaatactagtattaatttatgtatctggttaatgacatacttggagagcaaattatggaaatgtgtattttatatgatttttgaggtcctgtctaaaccctgtgtccctgagggatctgtctcactggcatcttgttgagggccttgcacataggaaacttttgataagtatctgcggaaaaacaaacatgaatcctgtgatattgggctcttcaggaagcataaagcaattgtgaaatacagtataccgcagtggctctaggtggaggaaaggaggaaaaagtgcttattatgtgcaacattatgattaatctgattatacaccatttttgagcagatcttggaatgaatgacatgacctttccctagagaataaggatgaaataatcactcattctatgaacagtgacactactttctattctttagctgtactgtaatttctttgagttgatagttttacaaattcttaataggttcaaaagcaatctggtctgaataacactggatttgtttctgtgatctctgaggtctattttatgtttttgctgctacttctgtggaagtagctttgaactagttttactttgaactttcacgctgaaacatgctagtgatatctagaaagggctaattaggtctcatcctttaatgccccttaaataagtcttgctgattttcagacagggaagtctctctattacactggagctgttttatagataagtcaatattgtatcaggcaagataaaccaatgtcataacaggcattgccaacctcactgacacagggtcatagtgtataataatatactgtactatataatatatcatctttagaggtatgattttttcatgaaagataagcttttggtaatattcattttaaagtggacttattaaaattggatgctagagaatcaagtttattttatgtatatatttttctgattataagagtaatatatgttcattgtaaaaatttttaaaacacagaaactatatgcaaagaaaaaataaaaattatctataatctcagaacccagaaatagccactattaacatttcctacgtattttattttacatagatcatattgtatatagttagtatctttattaatttttattatgaaactttcctttgtcattattagtcttcaaaagcatgatttttaatagttgttgagtattccaccacaggaatgtatcacaacttaaccgttcccgtttgttagactagtttcttattaatgttgatgaatgttgtttaaaaataattttgttgctacatttactttaatttccttgactgtaaagagaagtaattttgctccttgataaagtattatattaataataaatctgcctgcaactttttgccttctttcataatcataaaaaaa

SEQ ID NO: 3 - DNA, 50081 bp, Homo sapiens
ttttctccccgacgaagccgctccagggctgctctcagaggacgcgcggcaggcaaagagaatgaacctgagcgtccacgaaacgtcctgcacggctcccgggagctgggagaaacaggtgcctttctccgacgtccgcgggcgacgcctgccgcaccttgcccgctgccgcgcccctcccgggcacccctcgccctcggcgcccctgcccccgcccccagtgccagggcggaggcagtcccggctcgcaggtaattattgccagcggagcccgccggggagcgggggtgggcgcgccggcggtgggcgggcgggcgcggcggggcgcgggcataaaggggcgcggcgcggggccccggagcctggctcccgcgcagcatgcccgccagcgccccgccgcgccgcccgcggccgccgccgccgtcgctgtcgctgctgctggtgctgctgggcctgggcggccgccgcctgcgtgcggagccgggcgacggcgcgcagacctgggcccgtttctcgcggcctcctgcccccgaggccgcgggcctcttccagggcaccttccccgacggcttcctctgggccgtgggcagcgccgcctaccagaccgagggcggctggcagcagcacggcaagggtgcgtccatctgggatacgttcacccaccaccccctggcacccccgggagactcccggaacgccagtctgccgttgggcgccccgtcgccgctgcagcccgccaccggggacgtagccagcgacagctacaacaacgtcttccgcgacacggaggcgctgcgcgagctcggggtcactcactaccgcttctccatctcgtgggcgcgagtgctccccaatggcagcgcgggcgtccccaaccgcgaggggctgcgctactaccggcgcctgctggagcggctgcgggagctgggcgtgcagcccgtggtcaccctgtaccactgggacctgccccagcgcctgcaggacgcctacggcggctgggccaaccgcgccctggccgaccacttcagggattacgcggagctctgcttccgccacttcggcggtcaggtcaagtactggatcaccatcgacaacccctacgtggtggcctggcacggctacgccaccgggcgcctggcccccggcatccggggcagcccgcggctcgggtacctggtggcgcacaacctcctcctggtgagtgcgaggggccaggcggagggccacgcaggggagacagagggcctccacaggggccagggggaagtgtgggaactgagtctcccccagacgaggcttcacttggacacgtgtatgtggtcaccgggggaaactgagcagttctgacttcccttggaaggcgtggaattaggagagaaatcccttagtgggcacacgagtgagtgccccttggagtccatctgtggaaaggaagcggtgataggtttccgcagtgaggaaagaaactcctttctctgggtgtagaggaattcctagaggtgagggcggggaggatttgctaggagtaactgcaggaagagaatgagcagggtggatgaaagaaacacgtttgttcttaagccgcacagatagcattactctctggagctgtcacgagttcagtgttaatccaataagatctgtcttgcttgtggcacaagttcacactgttgtgaaagtgtcaaaacacaactcccggagagtcagatttaaactgtgtttggaggtcccttctgcggcagggcagggaactgcatccaccaatcttattcaccggggtgtgaagaccgctttatacaatctcaaacaaagctagtcatagaaacacagaaacaactgcagggtgatatagattgttctaccacatctatatttccacagtgctctctacagttgaaagggttcctcacttatgacattgttagcctgataaaattaattgtaacacccttgtgcaaaatattttattaattttgacagcaggaaatattgctatgccatggtaaagttgtctcaagtatttcttcctccttttttctccttagtgttcaaatttaggcaccattgttttggggattcatttataatcatgtcagttgttagtacactttgtacagagttttgtatcacaataaaatattttatgtacatgtataaacattttaataaaagtgtttaaatgtacaacttttggtacccaattctgatcacttgtgggcctactggaagacttatttttaaatttagaattcagccacatattctgaacaaagtcagtactaaaaccatttctaaacacttttaattaaagccatagataatatgggcagagcagtactgggttattgaggtgacttagtgctctgagctgctggtaatgttgtaataatggctaaattagattttatgtcagttccacaaaacctgtttgctgctactggccataaaagaatgttggatttgtttggaaacacattgtgatttttgtgatttcatacttatgtatgtcttttcataaggatgttttagagcggtgattcttatactttttcaatccttttatggatctgaaaaactctaaagaccaccagtgtatggctggtgatagaaaaactatgggaagaatggtctcactttttgaagaaaacataagcaagttatcagttcatactggaagcaaataaatggatcagctggaggatctaatagatcatgcccataaaatctttactttagatttaaatcctacaagttctatttttagagtccctaggataaacttgcatatacaaagtagtcaattgttattatagtgacttataggatcattttattataagataaagacttgcgtgctctctggtgctctggtgttttaaataatcaacatgttttagatctttttgcccactcatggccctctgattaacttctcagttatatttttcaatgcttacacaaaaaatttgccaaacactaaccatccttcctagaaatgcagtggactttgtatttagggaaattagtaacaggtgaatgctccatttgctgatgttgcaaacaaactgttagttggtgtaaagtattaattttgctttcacatttcttttggtttggtgtttagtcattcttgtatcttatttagaagcgagttcccattgtcaaagaaagtatcttgaattaattttgttgtatgagatactttcattctgttttcaatgacctttctcggtgtgtatataggggttgggggaagcgagaatagaaaaatttgagcttacctgaaaaagataaaacattctgcagattttgataaaagggcattttagctggggcttagatattgctgctgtacagatataggggccagttgtgttccgggatctgatgttggtgaaggataaagactggctagcacccttccctcactccctctgtgtttccactaacttacaaatgggctaaggagatcccctgggatgatgggaagcttgacccctcctagagagtcaagaatcattgatggcctggaggacaggaggcagaacaaacaaacagcaggacaaataatggaggccctgctcagaatggaggtcttggtgttctttgtgggttcctcatactggagtgagccttggcaaaaatggtgtgtgtgtgtgtgtgtgtgtgtgagagagagagagagagagagagaggagacagaagagaaggagaagaggggagaggggaggagggacagagagagagaggtctcctacaaagagcttggagctcaggctgctttgctcatttgagtgtaatccaggaggatgcaccttttctctcggacttgaagaccaactgctcagccttcacagctcttgaaacaaagttcttaatgtcccctgaaatcagcactagggactgtagttggggagttcagccccagcagtgcctctgagtctttgttctgtatatgggttctaagaggattagaatcaagttggaaatacacgtgttcccatttcccaccttcaactcccctcaccgcctgagattcttgcaaacatttgtgcaaggaacctgtgaaaaagacttctttcacgtaaggaagtagggctaggagatattaatctgggcattgttgagcattagcatcagtgtgcaggtctcagcccatctgtctgcaatgtttgttgaagccatcacttcttgtcccttgaatccttcctcaacttggatttcaggccctggctctcccttgcttctcctctcagctccctggcgattccctcttagtctcctttgcttgttcctcctcaccttgcctgtaaatagtggagggctcagggctctgtctagattcactccaggccattgctttgaatactgctgcatcccgatgactggagattttgagtctccacccaggtttccccgagcatcagactggcatagacaaccgccttctccacgtggatgtctaatgggcatgtcacatcgaacacatcccaaaccaaacccctgcttccccttctctattatgatgactttttactcttccagttgttggaccccgaatcttaactactcccctttttcatgtcttgtttctaatccatcaacaaatcctgcagctctacctacctccaggatgtgtccagaatcaaaccacttctccccttatctatgactacgatggaggtgcaagacaccatccaaccttgcctgggtaatgggaacagcctcactgctggggttcttatttctgccttgccccctgcagtgtgccatgatggatgctctacagggtgacattatgtgtggacaagacagagtccttgctttttcaaatcccacctgttcttctggagccagctaacacgctcccttgggccccatgcctgaatgccatctcttccttctatggcatcctgcacaggacttcgctttgctcagatgcctcctcccgcgctcttccctttatttaaccatttgtctccattttaccccttcgctatctggggtaatcctgttcccttcatctgtatctctgaaaatggcatcttgccctcccttggtgctcagaaggtggcttctggtttaccgaatagcctgatttcacctttataaactcatgctactagaattttctctccttgtcagtctttctagccactttccaatggaacagagactaccaaatcctaatctgataaggaaaaataggtaaacataacgtggcagactctgacatctgcagtgaaaatttaaggttcatcatcaaatgtatgattccattgtgaaaatgtccagttaaaagctatctgtagtcaccatgcagcattatgaagaagttttcagaataagggcaggtggtaaagtctcctggccagctttaggagtatatattggaaggggctttgttagctgattgaatttttattccccaagtaattagtatgagcgatttgtctactgtatatatgctcaaattacatatactatgcacttttgagaaatactttacaagtttttctttcattaaaatgtatttggggccgggcgcagtggctcacacctgtaatcccagcactttgggaggctgaggcgggtggatcacctgagatcaggagtttgagaacagcctggccaacatggtgaaaccccgtctctactaaaaatatggaaaattcaccagacgtggtggcaggtgcctgtaatcccagctacttgggaggctgaggcaggagaatcgtttgaacatgggaggcagaggttgtagtgagctgagatcgcaccattgcactccatcctgagcaacaagagcgaaactccatctcaaaaaaatgtattttgtacataactagaataatcaactggattgaaagttaatataaattttagaatactactgaattcaatagtgcatttagtcagaaatagttaaaatatctcccaaacagcttgaatcactcctttttgaacacattgttttttgaaggttacagtcaagtccaagaaaaaattttaaaataggaaaagaattaatcaaatcatttcaaaatcatagcagtgttttaacaatgccaattattttgagttgaagaaaagaaggaaaatgtaaactatgcattcaagtaatgtcaatgcaccttctcaggtgcaaaatgaatgaacaattaactgtgccagctgaatatgtagcaatcacggcttttgcacatagaagccttctgaattatcttatattctcaaaaatatcattgctctaaactagaattttattataattaatttgtgcaaaagttgtctttctttcaatgaatgttgtctttttgaggcataaaggaaattttaggtggaggaggagaatagaatacatgcttactgggaggacaagtaataataggtgagtaaaaacacacagcttattgaattgtctcagatccacattttctccagaaatgcaaagttatacttgaaatctatatttaaacagataagcagaatgtgacttttatatgctctattttgatgtattctgagtatgaagataacctgaaggatgcctttttcccctctttatcttggggtaaactcttacttacccttcagagacttgactcaaatattttcattctgcaataaccacaggtagaatggatcatttactcagttgtggctctcttaagagcacagactacctatcatatttatttcaaaatttgtagtgcttatctcagtgcttggcacagaggaaagaaatacaatttttaataaaggcataagtggagaggtaaaatacatatttctggtttcaacgtgtccatattctttcttgtaaatggaatattgcgcttgcagtctgctctcacacaatatctgggaaggagttttaaagagtgaggaagaacagtaaagattttcctaggcagatacgttatgtgacctttgttgatttccagagcacataatatcccaatttcatccagtggacaggaaaagagaggaagagggaaaaggtggcctctcaagtgcctgacctggaagctgcacctatcactgtcccttgggcagaactcagtcacatggtcaaaaggagtctgcaaataattgtcccctgaggagccgggtgctcagtgaatgattctattaagttgaaatcagatgggaacagatcatttaggataaccagcaggctctaccacattgcctaactcccaggcatattgtgagaattaaaagcaccttatatatgtcaacgtgttttgaaaaaatacaaaaagctctacaaaagtgagctataaatttatcattaataataatagtaataatagcaaaatacttgagaaatggtcctcttgagctgtttagaaggaatcatacaaatgcattagacatggtagcctcacttaactacttaatttgcctcttctttgaaattatttcaatagcatttgaccaaaaactatcaaatcatttttgaaataacgtatttttacataaaacacattatcaaatatctttctggatccagcttggtggtaaaaagatacatactaaagtttatgactgatataactttatatattgactaaaccaaagataaatattgattgaacttttgtggccccagtttcaatagatttttttattaacattttttgtttgaagtacagatgtcacgtcatccatgaaccggtatcattatagcttgataaaatactcaaactgaaagcagtgatgtacattaattttaaatataatggttaagcaaatgttatttccatatctataagtgcattttatttgataattagaatgttagaatcagaaggaatttggaaaatcccaggttacacttctctccaggacgaactccttacagtggctctcctggggtcagctagcttttgcttgaccctttgaagcaggaggaactcagttccactcaagttgcccccttgattttctctttcatattgaacctgctgctgcacaggactcctccgtgactgtctctttgccttcctaaatctcaaagaataaagcaagtcactttttgacaagacatctttcacatatttgaaaagacatctttcacatatttgaaaagaccagtacgttttgttcttcctagaattttctccttcatgctaatgtctgctccaagaagacatggaggtgactggacatttagtcaggaacatttcttgagagcttaccctgtggcaggcaggtgcaatgttgaattctgagaatatgaatgtaacctttgaacaggtggctctgtcttctgacgtttcaggataaggtggaggagaggagaggggaaagtcgacctatctccccagcggggagggtttagggttgtctgtgggacctgcctctgtctcatcatccatcaccattgtcctgctgatgggtgcagaggactgaggacgagtggttggagttctccctgtgccggaccctgtagagagtccagagccctggctcccaaaggaggcatatttgtagggctcttttcgtgagggtccagaggacggctgcaaagctgcaggggaggatgggggcatgggaggcaagcccgttgccctgtcagagcctcttggggatgctgctcttccccgcctgtcctctcttctctttgatttctcatcatgtggtcctctttcctctgccttttcctctcttcggtgtcccaagttcttccatagagacctgtgtccccttgttcccatcaggcctttatgccagccctgcacaggtgcggggacagtggtaggggcgtctcactcccgactcaactacattctcccagaaattgtctgcagtcaaacacaactcagcgtggacattgctcaccaaaggtatactgcttgggctgcacgagagattcagttttctcctatctatctggcatgggctgtgagggggctcctaacctagggggcctttttactccttcctggccagagctgccatttccaagtttctgcactgtcagaaaagagggataaggtaagattcctgccctcatgtcacagattagtaggggagaggtaattgtcaaataattacagtaaaatgtataaatgccttaagagaaataagtacttgcttaggaacctatttcaagaaaggagagataggttagcgttttggggaggtagtacgggagcagtcagggaaagcaatacttgtaagatcagaggctggcaatctttttctgtagagggctaagttttggactttgcagatcctacagtgtctgttgcaactattacacagttctgtggttgcagcatgaatgcagccatgggcaatgtgttaatgaatgggcacagctgtgtttcaataaaactttatttacaaaaataggtagtgggccctgctgctgggcttggcttgctgggcacacaaagtgagaagggcattgtgcctttctaggaaacatcccttgcaaagtcagtacaggtgagacagtgtggccattcccaccacagcatgaggtcttttgaatggctgaagcgtcaggtgggaaaggatgaggagctgaagaagaaagcctgggaatttccaaggcctctcttaacatttgctgtgcagaaccaagcagtgccccaggtgttcagagtaggcttcagcgattacatttcttgttagacactgaattttgttcatgccgcccaagattgcattaacaattttggctgtcaccttaccgcggactcaaattagtttgcagtcagctaaaacctctcaggttttttccccccagcttgtgaattgctgtgaagccaaacatccctgtcatctctttggtagtttattttcaaactttcaatcttattaaatttcatctccaatgtgcaaagataagaggaagtttaccgctgatggtggtttattctttaaaactggacatcagctaaatgtgttaaggtaatccgttagaggaatgttggtacgcttatacagtggaatactaattggttattaaaatgttgatgtatgattattaaaaggcagcccatgttatgtctcttctgtatgaataagtatgtaaaaagtatgtaaagattactgtatagttcatgggtgggactgggctctctctagcctctgtccttcattctccctgccagtgccttttcccttgcagtgtcccttccctggctgcatactcaagactggatttgcccagccaaaccccattaactcacagaagcaagaggaaaaaaataaattcttattagttcaattctccaaattgggggcggtctatttcacagcaataggtaactgacgtaaaaggaaaggaaacaatgaagtaattccagaaaatttcctatagctggaggacatgcattttcatattgaatgggaagaatggggtgggggggtcatacctctgtgttattgcagggtcctggctcctagggacctaggcacctcttcagtcagtgaattatagcttggaaaattggctaagtcggggaactgtacaaggggtcatcactttttatgtagccatgctctatgaaccatctttataactccctggcttctggcagttaagcaccactcactatctggcctctgtaatttcaacttctgaccatccccgtgggtggcaacaagcccagctctgtgacacctctgctaccagcagccctggcctggagagcggtgcccccactgaatgtctcagtgagattggcacccccatgagcatattcctgagggtgcagtgaatgctctttctccctacttgggacttctgggaaacactgtcctctggtgggttgtttgacctcacataatatactggttcttgcatgccagcttctctctgcttcttgattcttttatcagctgttagggcaacaatgtcaggcatttccgcctgctcagcattgggcatccctttttacaggcttctcagagccaccctagcagcaactttaccgcgtctcctggggtccttgctggatcgtgaaaacatacttccggagaaagtgttccctagataatgaattatttcttgtcctgtgttaagttccagttccttggtcaaggaccccagcgctactcctcaggatttgcagtgcatgctcaatcttgaagcaatgttgggatatagtatcttttcttctctatcaggcccagctgtctccaggtgggttatactaggatttaatcctcgttagtgccgggtcagctcctgaggggtgcacctgttgctttttagggacgaggcctcttcagcataaagaagtgctatctcttactagggaagaatgggccacctctgtaagctccaaatattcccaggtttccccaaccggggccttgaccttggcataactgatctgccttggctgagcatttaaacatccctaaatctctgcaagtctatcaggtcctaggcctgctgctaagtttctctactcttccactgtaggagagaaggctgtggctcttatattgagcctttaactgtttgttattggccctcagtttctcattagctctctgtagagtctcaatacaatatatcagaaagcattcaattccatctccttgtaggcagggaatgcctaaatcattgcacctacaatgacattctccaccaggatatttcccaggttactgctgttgaaatatttagcaattgaagcaccatttgtgcaaggggactatctgcccctacacaacactcagaatggcattctctttgctttccagaaagtgaatgagctggttccccaacccctcttgtgttagtttgagtcctgaaagaagtaaatgtcaagataggattagatgtacaagaaatttattggggaaagaattgtgaaggataaaggggaaggagctaggggaggcattgaacctttggatcatgatgtgtgtccaacaactatgaaggagagtgggcagggagaaggatgggctgggaagagtttcagttggcactgtgggtctcagaacaacccaggcataggctgatggggacgccttgagccaatgttgcccattggcggagtccacatcttgctgaaatgggcctgcattagttcccctgccatgtttagtcatctgggagtagccgatgagaatcatgatcttaggatcaactgcaatggcagattcaaaggggtagcaactgatggcctcagtcaactgtgctccttatagcaggaacactgagcagtgcatttcttggccatcacaaagactagtgaggagtgcccttcagagaagggaatgaaaattatttccagcctagaatttgatacttatctaaactgtcaatcattcatgagagtcaaagtcccaaaaaataaatcttccataaaccctttctcaggaagttattggagggtaaccgccacataacatgaggaagacaaggaggaagacatgggatctatgaaagggcaggtctaacccaggaaaaggatgatgaactgtagacccatgatgtcagatgtgcagcagactaagatcagccagtgcagaatgggggagattccagaagtgtgtccccccaaaatattggaggctcatgtgacttccctggggaagtttctgctgagaggctattggaaattgagggaagaattagccacagtcccaaagaaaacagagccaatcaaaaagcaatgcaattatgaacttcaaagaaaacaaaaataagaaggaaaacagtcttacttactacattacaaggtccagctgtgaatgatattcatgttggcagaataatgtcaataccaaatgtcggttaaacccaaaactattgtataactaaattgcctgtgtaagagagctaaacccttatctagcataataggaagtcagtggatacttcctgaagtgtggatgtgtggagatgtaaattccaaaagaaaacgtcttaagagtcaaaagtggttgcctctagagagaactgggggaattgtagggcaatgtaggacagggaactgtgatttttcttgtaagttgttctgacattttaaaccatgaacatatattacttgtatttttaattttaaaaagacataaatattttttctatcactttaaatattaattttgctagttatagcacatatttatagcattacactctgtagagctcttttggattcaagttttggagaattttcaaagttttagattaatgcctttgtgagttttcatccctttgatgataacaagttacaaagaaatagggttatgaataaaccttgtgggatattgttagtcaccttctctcaagtcgatctgtcccatgaatcaatgtttcaaacattgaccagcatttgatacttaataagcaacgagtaagtttttgttgaaccaatgaatactcttaaaatatattttttcaagtggcaacagtactatcttatttgcactctacttttctttttgaccctaagaatgtcacaaaaatgtttagcaactgtcaagattattacatacagagatgactattgtgttctcagatatgctgtatgtcttagttcattttgtacagctataagagtagctgagactaggttatttatttatttatttatttattttttgagacggagtcttgctgtgttgcccaggctggagtgcagtggcacaatctcggctcactccaagctccgcctcccaggttcacgccattctcctgcctcagcctcccgagtagctgggactacaggcacctgccgccatgcctggctaattttttgtatttttagtagagacggggtttcaccgtgttagtcgggatggtctcgatctcctgaccttgtgatccactcacctcggcctcccaaagtgctgggattacaggcgtgagccaccgcgcctggccctattttttttttttttaaaggaatttatttcctcacagttctgttggctgggaagtccaagggcaagtctctggcatctggtgagagacttcttgctgctttctcccatggtggaaggtgagagggcaagagagagacaaaaggaggctgaactcatccttttacaaggaacccatgcctgagatattgaacccacttctatgataacagcattaattcattgatgaggacagagacctcattgcctaattggcctaaagtgagacctcttaaaggtctcgcttaatactattacaatggcaattaaatttcaacatgagttttggaggggcaagcattcaaatcatagcaccatataatacaataaaaattttctgagctaagtttggtaaatttattctaggaatccatgttgtctcctagttatcttcccttccatagtactgatgcatttttgttaaccattacctaattttgcttgagtctattatttctgaaattcacattatcttcccttttaaagttgagaaaattttcattcttcaagcgccttattttctataatctctcaaagtaactgatggctgttgcatgatcataagtgcaaattattttgctagaccacacttggagatgatgaatttggaatggcatgcagactcccgacatcaggagtcttgtctcctgcaataatcaggaacccaggcttaaaagggagcaggtacaacagaagggcaaggggtgacaatgctggtgaaagacatttgaggaaagctatcaacatgaaacagaataaaataaacagaaaagcaaaccagggaaaataaattatgcaggaaattaaacacatacacaaactgaaacaggaaccatcagaacatataaaaaattcttgaacatcagtaacacaatagttgaaacaaaatatttagtaaaatatttgaaaagtaaggtcaaagcaatgctgtaaaagtagtacaagatgataaagaaatagaacacatttttgaaagggaaaaagatttaaaggatattcataaagatccaacatcagactaatagaagttctggaaagagagaataaggaaatacaggtcaggaaatttgtaaagaaataatataataaaatgccccagaactgaagaacatgagcttttaactttaaagagccgactgagttcctagctcaatgaatgtatagacatgtaagggtattgaaagttcaccttcagtgtcatgtttcctaagaagctaatggaagatatgcaccagcaaaattgtagtaaatacataggaagaaatagtatatagaaaaagtatggtttcaatccataagaaataaaggaaactcccaggacgagagcagctcttgacagcatctagtccaaactggacttggaggctggaaacttctagcagggaaggaagagctctgggtgaaaaagtagactcaacagaatagatacgatcatagaaaacatgatagagaatcactaacacattgaaaaaatcacatatagaatattctgcacgcttaataatgaggtcattatttattcaagggaaaattaaaagctgtttagaaaagggaaatgttatagtgccctatttggctctaaaatgaacatttatataggaatcttcatgtaaatactaacaatgatttaaataagaacagacatttcggaaaataagggaagaaaatggggcatgtaaaagagctaactcctcattatcctaatgaattattaaatttcacaatagcatagtatttagaaatatgatagttattacgagaagaaagagctaaaaggttgccagtggggagcaggagtgagggttagagacgggattgggggagatgcttactgttttcatgataagcctttggtactatttgattttaaactatataaatgcatttattaatttaaagtaatttaaaaaacccataccactggataatgcttgataatttctagagtcctttttttttgtattttggggcaggtaaattcattgagagacccagagagtttagctgactttcctgtgggtaccaagggtcagagctggggtcaaaactcaggttttctgaacccctattcccagtgtacattccatgactccaggctgcctcccgcattgcacaggttacatctaggggtgtgctagcaaatgcctagaccatcctcgtccacatcagcatctgaaatggacaagaatgttagtcatgacttgccactaacgtctttaaccttaattgacatctgagagtgtcatcattacatcattacaaaaacactaacccagatacatctgttcccattactatttctgcgtaattcccccagacttaattgctttaaataaccattttatttggtttacaaacttatgggtcaggattaggggagggctcacccacgcagtttttctctggtccgcagtcatctgaagcttgaacggggtggggcatgcaagacggctcacacatgtgatccgcggttgagcctggctgtggaccagagcatctctgttggcctcgcttacacaagtggtctcagattagtagcctctgtacatggaaactagcttccctcccggcaagcatcccaagagaactaggaggaagtggtatggacttcttttcttcttcttcttcttcttttcacttattcttgatatcatgtagcttcatttctatcagagcagtcacatgcccacagattcaagggggagggtccacagatccgccaatgggaggaacagccaggttatatcgtaaaagatcatggggcatgggagatacccatctattttctgtaaaaaatacattttgccacagcatcactggctttccagctcacagtgatctgcctggaatgcctttttccgtctctccaggatctacatctttcaatataaggtttagaaactacctcctacaggaagacttccttgatttccctcagcaagaaagaatctttccttcctctgaatcgccattgcattaaaaaaaaaatccatcttctggcgttttcactttcagacttatactgtaattattctatgtacctatgtttgacccctcacgatgcccagcataataatttgtctattgagtaaatatttgcttaatgaaataatcattacatgataactcaaatagcagtcctgaaaaagtgcatttcaattcagatctctcttttttcttccttcacaattctcatttccgaaaaatgaaagaagccagaggatcctttatgaggagttacagtataacttatgcgtggctgtttcctgtgtttactgctactcagtgagaaatacgggagatgggagagtgaaaaaccatgtcatttacaatttgattaaaaagctttttatcttttcctttcacgtttaagccttgccgttttaaaaatttccctttcgtcacaggggatcaagcagcagttaacgctgcagttccctgttctggaaacactctcaaaggtgtttcaacacattttgtctcaactctgactcctgccccgctgccccacgccatccagccacactgaaggtcttgcatttgtgccttggggcattatttttttttactcttctccctgcctggaaggttcttcccaacctgccctaccgccatagccacagaaccaacccttcactttccttaagtctatggtcacaagctccttctcaaaggaggcacacctctaaccacccccatttggtgacttcctgtctcctggaggcccccggaactagtcttctctacttccagcaacccactttacgagcagcggagaagactgactacttctgggggcctcgaggagacaggaagtcaccaaatttacaggtgctgtggatgaaactgtgtgataatgaatttattgggcttgtttttttagcttctgaatagaagaaccaataagatcatttttttaaaaaagataaaaacagacaaacacaaaccctctagtataaaagcatttttttttaaaaaagatgaacacacaccctcagattgccttcttttgaaaaggcaatctgagattccttatgaaatccccagacagaagctgtttctttgaatttaatatgctgtacactgtagagccaagaggcttatagagtgttaattaacaccccttgtcaaacatttgtaaatgaatctggctaaagctcaaggaaaccaggtttttctgtgtgataaaatataatctttggagattatttatgatcacaaagggagactgtacagaaaattttcctagacttggaaatgaggcaggattattggtgtccatctggactgaggcctcaggcaggcctgcctagttaatcccattctcctatcctcattctctgggggtgaagaaggcagtgcacctttgttcaatttgctgcttaccatggattagggcattttaaattctgtaagggtagtttttaacttgtagaaaactgatagcgatgggaaggattcttgcctaatagggacacaaacggattttgttctgtagagatgtaaatgaaaagatgaaaatcacaacacatttaatgaaaggaaaataaagactcttgtgagtgccaccaaaaaaaaaaataaataaaatgtatctcctctccgtggcaccccaggccttccttacctcactctatttcttcatagcacttattgccttagaacattctttataatttacatttattatgtttgttgttggttgtcgctcctcctgctaaaatggaatctccgtgagggcacgggtttttctttgatttgttccctgttgtgccccaaagtgctagaatcatgccaggcacacaatagatgctcaataaatacttctggaatgaaaatccccctccacttagaccagtgagttccaaacttttttgatcttgacccatagtaagaaaggcattttccatgttgaatatacaccattgaaacaaaattttcacagaaaaaacttaccattattacaggcagtgcactgtgatatttcctaatctcttctattttgattttcaaaattgctgaggctactcataggttgatttcacaagtggagtttgtgttatgaaaaactttgacttaacaccatgtcgaaactgctaccacaaaaaggcaaatgcgaaagaagggggaaagagcaggccgatgactttccgctatccaccactccataaagcatatttcattttcctgtaattgtatcctgttcaatgatgtagaaatcctcacacactcacatgccactttttctttgggtgaaaagcgttctctactgcaagatgaattgagttatttcaaaagcaaagagctataaatgagcctgttaaagaaagtctcaaggagagtctgttggcatctgctgttgataacttaaagcaggagaattagataaggaggcagaagtagaatgtttagaaaataaaagtgaccattatagaggaaaaactgcttgtctcagttttgttgatattggagagtagctctttcatgagggtattgtggaattattggcaattatactaatagatgtttactgaaaaaatcctatttgactgatgaaccatggaatacttttgctgaccttgtggaaaacatcacttatctgagttccttatcttcttgtctcttttttctcatctagcctatgcctccctacctgttccctcatggttctcattttgttgctattagaaaaacagagatacaaaaaccaggaattggaacccctctgtgtctccagtaataattctgtgattaatgggttagatggattctggtcacaagctggatattttgttaacaacctagcgtcaatgacttggaatgattttcaccgcagcatgatttagtattgaatagaatgatttaactaatgttaattagttctgtacagataaattaatgaagcaagaagctcaatctctgatttattgatgtatttacccagtgtaagttatgaaatcttttttatttcatttgaaggaagtttttatttaaatacaaataaataagccctttattgtcacctactttggaaaagtccagataaaacaatcttaagtaacaaaactccaaaattacaacatgattttcaaaaactaccctgacctttgtcttgcctggttgttacagtgtacttttaactaaacggattcttatagaatctcaagtttggttatatttgtattaaggaactctatatttgcatttgaccagccctaactaaaacaagcttaaggaagaaaagggactttacgacaaggacaaaggattgaagaagaactccagggactttagatgttaacaactgtatttgtcactaaagtaaccattactgtcaaggtgggttctccttctccctcactaaacacatgcacacacaccacacacacatacacataccacatacataccacacacatgcacacacaccacacacacaccacaccacacacacagaaatacacacagacatacacacatgcacttatgcttgggtcagtttggcttttatcatcccagagagtctcttcccatgtcagcaactccagattcacttcctctcctaatataggacagtcacatgtctctattccagcaaaacaatctcagggaaggtatctcattggcctggcttgggtcattcaacttgaagtacaagaagagaaaacagtattcttttatttttatagatttcctgtaaaatcagtggataatggtaaaaatttcatgacagcaggtgttttaccttatttatgtttactatctctagaacccagtatgatagttcataataaatgcttactaaaacaattatcaaactgtacacttaaaaattaaaagagtaaattttaagttgtgtatgttttaccacagtaaaacaaaataaaataaacctattattgatcttattgtctatttctcaaaagtagcatacgtcataatttcatgtgatttctaaaggagatctaatctcaaacttagttcttagagtaaaataaaaggttttggcaatcatatacagcagtagactttaccttgaagatttaacaaaggtttgaaagcaaaatgctatgatactgaatatactaatgtttgagggcttgtgaaaaggtcttaatataggaatcatatatcttcttttagattgtgtcttggggaacattgggggtcagtttttctatcagtgttagtatgtagtaagtaatggcatacatctaagcttgagattttgctcatttctgtattcttagcacttatatatccaacatataataggtgttggataatatttgttgaataaatgactaaataaatgtcttggcatgagaattatgccatctacaaaaccgtcacttttaaaaaaaacaacaaagatttttgaacctgtagatccagccagaaagcaagaaatatcttcacctttccagactgacttattttttggtctagctgtgtactatgcatgagctgtcaactttaatactttattttttaatgcctaggtctagtgagttacaatgtgatgtagactgattgaattaaaggccccaatgttgtcctctcttcattgtatccatgccttttgtcatgtaactttgcagtgtcctctactctaggtgtccattcagcttatcctttgactctggccttcttctcctttgactctggctttatccatgtgacttactttagccaacaatcataatgctggcaaacactgagaaaatgcttattagtgtctgcttagtctcttgcttctctgcaattgccatgagaaaatgcccaagctagtacactggaggatgagacatgtggggcagaagcaacctgtcccatttgtcccagccaacaccatcctagattaaccagatgagaccagagcagaagacatgttcagtggagcccaacccagctcacgaataatatttcttcatgagcatgtgagcaatgtgtaactaatgcaccatgctctttttattctgcacttccaaacatctctcagattgccctgttgttttgcttttcagtagtcatcacctttgtccagaaccttaattgtatctgcattttggcagtatcctgccaattttccacatccagctgttcacattctaatgcattctgcaatggatgattttgagcatatgactgatcatcactgtctcagaaatttatgctaatctattgtgacctattctgtcaaatccaaataactttacattcaaaacttttcatattctggccccatactgtctattgtaatcttattttctaaaagccacaaactattaatcctgtagcctaattaggttgatatcatcattttcacaacacgttgggtaaattcttatatctgttcctttaatcatagtttccatgcttagacactccattgccctccctaagcaccagccagagtccactatggggggatattttaaaggctcttttcccctaacccatctgccccagaggtggcacaggctagaaaagatagtccaattatttggcttaattatttggatcttacagtttttttgggagagttccagatgatttgatgaaatgcaacatggtcaaaaacattttggtggagaataatttaatcaactagaccatttggtgaaaaataaatcccagttactagacttcccagtattaataattttggtatgccaggaatttttaaaacatttctaacaaattttttcagaaatcagaatgactgaaaggtataacaataggaaattaggaaatcaataagtaatcattgaaaagtgagtgattgaattagttggaacaatctatgccatccatcctgtgggggaaaaaatgaaaacagctaaacaagcagagcagacatcatcaacttagaaaaacaatatagtaaataatttgaactagaaaattttccaaaattgaaaattccagttttaaaaaatttgtgttttcttattcaaaaagcaatacatatttcgttacataaaagtcagaaaatcagagaagcagaagaacataataaatgcccactgtcctactcaatagagactataactgctatctctgtacaaatcctttaaaatcctgtcttatgtgtatttattttacatctgtacatatgattccaaaattaggtaatactgtacgtggtctatctatagctaactatagtttgtaatttaataagctatatttgtccatggtgatcatttattgctcataaaattaataaattataatttatttatcaacactgatcatttgttgctaataatttatttcatttaatgtcttattcctgggttattttaactaatccctattgttagccctttaggttgtttcaactaatttaagaattataaatggcccatagtagttacttaataaatatttgagtgaaaggataaatgaatgaataattccacattattggatgttcaagttgtttccattcaactttattgttgtgggggagtataatacaatcttggtgaagagccaccaacttaggaagaactatgaagaagcatgttatttactcctgcttgtgggtggggtgcattgggaaaggaatgcctaaatgcacgtggggagccaaacaagacttcacaggggagatgggaattgagccaaatttggaagaagtgagaattttcagttgataattggggaagatagtactaagagtaagtggggttcattaggaactacttcttagaagaggcaagtccagaacagaaggcagaaagggatatggatatgaaggaaaagtagaatgcaacttcagggctagtaggttggcatccaacgtaggtacagccctggatgaggagtaatgagaaactgacctggctgtagcaaaacagctaggctgggaagtgatgctgtttacatttatatgcgattctactggtcttcctgttactttgtagaagactgtaaggcttggtgctcagagctagaaatgcaacttatttgatttgctaagaagtccaaagcaggtaggggtgccaagctgctgcctgttcacacatattcgtctaactgggtgtcagggaaagcagatatgagcccccttgcattggttaaatcctgagcaacttttaaggaactgcacacagagactactggctattttttgtggataaagttgtagatagttatttttgggaaattatttatgtgttctattaggtggttgttttgtgggaggctgtttgaaattctgtcttaaggaactgcagcttataaaccatagtgctgatagaaataagagaaataatttggtctgcgagcacaaacatcaggctagttggcatgcttatgtaaacaccacacagtagcagtgacttttaaggaagtcctcagacaatgtaatcccaatactcttcttgaagttgaggtaatagggtgccagaaagaaaaagaaaataaatctctagtgccttctaaaattttttcaatcctttgacctttttgaaaacggtcacctttaattcagaactaaagtaacatctggagggcccttttcttagatagggagaagacataaatgagctatttgaattattttctgcttatgggctgcatttcatttcttccaccattggtctcagtccatttaattaagattatttgtaggattaaattaatcctttaagaattattccatttcaatttttaaaaaatctaatgaatgatagataacaaaaagcggtccattcagtattacccctgaaacatatttgggtgattggtgataccatccatgtctctccaacttcagcttcatatactcagctgagtaccaggcatcttcattgaaagctagacttgtgacctaaatagaacttgatcccttcccttccccaactccttattttttgttttctttgagttccttgattcctcttcccctcctcacatgcaacatgtaacatccaattcatcagcaggttctacagattctgccttcaataatagcctaaatctagccacttgttactatcttttttggtccaaaccatcatgatcttttgcctggaccattgcaatagttgtctaactggtatccttgctcccattttgtcctcctagatcctattttccacgtagtagccactgatttttggaaacttatcaatcattacccagacacaatgagtacatttacatagttgtttgattccatttacataaagtcccacaatcactctatgtactgggaatcacaacagtggtctcctatgttgagaattgattaagagtcacaaggaaacaaggagtaatattccattgtattaacagaccacactgttcatacattctcctgtaaatggacatttatgttgttttcagtttttttgctattgtgattaaagctattaggttggtgccaaagtaattgcagtttttgccattactttttaaatggcaaaaactgcaattactttggcaccaacctaatactgtaagtatctttgaacttaccaatcaaacattactacaacacttgaataagaaacacttccatagcatttacaattctaagtgctttccatgtattgacttagttaatcctcacaaggatataaccatgaggtgctattactatccctactttacagatgaggaaatagaggcacagacttcagataatttgcccaaggtcccacagctaataagggggcagaattaggaaagcctggctgagtctatgctctcatcacttgctcaactctgcctggaatgcccttcccgtaccctctaccgcctctgcccaatcctccgggttctctgctccttccacacacccccatctcaggtcccagtcttttttagcattcttcactttttctgttacagatcctcagacaatgcttccttgaagaagacttgcctgtctaaactgccttcccatcccaatccctgttaccttctatcctttctctgctttgttttcatttgttatctctcttcttcccaagaatgtaagctccataagaacaggatcatattttgaatcccctagaaaagtgcctagtgtaatatttgttgaatgaatatgatccctgcaattaaaagctaaatatatatatttttttaactaacagattactgtagtagtttcaaagatgaatttatgtttcggagaaaatcagtattctcttgccacataaattgtaggtaattatatttctatacctgaatagctttgccaatgactaagatattaatctattatatatttattaatctatagatctttaaaattgatgcacatgttttataagcaatttgatgaattttgatgactacatacacctgtatgtgtatggatatctgtaacaaatatccaagtaaagatatagaatattctcattaccccagaaagtttctttgtgacttctaatcaattctcacctccgtaggaaaccgctgttgtgattcccatcaccacagactgattttgggactttatgtaaatggaatcaaacaactctgtgaatgtactctgtgtctgagttctttcaatctacacaatctttttgacattaatccatattactgcctagaagagtagttcactctttgcattaatgaatagtattccattgtataaacaaaccacactgtttatacattctcctgtgaatggacatttgtgttgttttcagttttctgctattgcgatttaagctactataagcatttttttttaattgtgatggggtttcgctcttgttccccaggctggagtgcagtggcagcgatctcagctcactgcagcttctgccacgtgggtccaagcgattctcctgcctcagcctcccgagtacctgggattacaggcatgtgccaccacacctggctaagtttcgtattttcagtagagacggggtttcaccatgttggtcaggctggtctcgaagtcctgacctcaggtgatccacccacctcggcctcccaaagtgctgggattacagtcttgagccactgtgcccggccagcatctttgaacatatcaatttgtaagttttatcttaatattgtacaagtcttttggggggcttatgttgtcatttctcttggtaaatacctaggtatgaacttgctagattatagagaaaatctatctttaattttataagaaactgtcaaatagttttccaaagtggtggtactatttatactcccaccatcaatgtatgaaatttccgttgttttacgtccttgccagaatttgttggtagtctttttttttttttgaggcagcatctcactgtgttgcccaggcatacaatggtgtgatctcggctcactgcaacctctgcctcccaggttcaagggattttcatgcctcagcatcccaagcagctgggactacagaggcgtgccaccacacccagctaatttttgtatttttagtagagatggggtttcaccatgttgcccaagttggtctcgaactcctggcttcaagtgattcgcccgcctcagcctcccaaattgctgggattataggcgtgagtcactgtacccagcctgttgccagtatttttagttgtaggcatcttagtgggtgtgagtgctcgttggggttttaatttgcattttcctgatagtgttgatgttgaggacatttctatgtgtttactgagcattggtgaagattctcttgtgaaatatctattcaaatattttgctcatggtgggaaggggagttatttttcttttactactgataggtaggcttacgtatttatttcggatataattattttgtcaattatatactaatcataaacaaaaactgataaattggacgacgtcaaaattaaaacctgctcatcaaatgttagcgaaatgtaaaggcaaatcacatactgaggggagatattttaatatatgtatatttatatagtgctttctgtgttctaagaagtattttcctactccaagataaagagactattctcttacattttgttctataagttttatagttttagcttttagctttggatctatgatctgtctcaaatttttatgcaagattggggtttaattttttcatacacttttccagttgtcaaggatcatttgttgaaacgtctttcctgttgccacataattgctttgatgcattcgttagaaatcagttggctgtgggtttattttggaattttctgttctgctcctttgatgtatttgtctatccttatgccaatatcaccctatattaaataattatagctttataataagtcttgaaatcaggtaatgtgaatgtttcaactgtgtttttcctttttctagttattttagctgttttatgttcttattgtatatattttagaatcaacttattcatttctacaaaaagtttattgggattctggatgagatggtgttgattcagtaggtcaatctggggaaatctgataacaatattgactcttccaatccatgaaaatggtatctcattctttatttacatattctttaatttctgttagcaatgtgttataattgtagcaaacttgcacatcttttgttaaattattttctaagtattttacgattttggtaccactgtaagtggcattgtatttaaaatttattttctgtttgttttctgttcatatataaatgcaattgattttcttttttttttttttttttttttttttttgagacagaggcttactttgtcacccaggctggagtgcagtggcgtgatcagcactcactgcagacttgaactcctgggctgaagggagcctctcacctcagcctcccaagtagctgggactatgggtgtgagccagtgttcctggccaaatgcagctgatttttgtattgacattgtattctgccaacttgctaaattaacttattcgttttaatagtttttctgtttttttaaaaatcttaggatttctacacagacaatcatgtttttaatgaacaacaaagtttgtttttttgtttgtttgtttttcccttttcaatcaacatgccttttatgtttttatttgccttactgcactggctaggacctccagtacaattttaatagcaatggtgagagtgtttaaatgtactcctgtgtatattttattcttttggaacttattataaatggaattgttttcttaattttctttttggactgttcattgctattgtacagaaatacaactgactattgtgtgttgatcttgtaccttgcaattttgctgaaatcgtttattttttgcaatagatttttgtgaattctttaggattttccatatgtagaatcatgttatctgtgaatagggatagttttacttcttttctaacttggatagttttttccttcctaattgctctggcaagaacttctagtacaatgttagagagcaatagtgaaagcaggcatcttcctttcaatcctgatgttaggggtgaagctctcagcctttcactgtaatgttggctgtggattttcataattttttgtttgtttgtttttttgtttgagacggagtttctcttgttgcctgggctggagtgcagtggcgtgatctcggctcatcacaacctctgcctcctgggttcaagcgattctcctgcctcagcctccagagtagctgggattacaggtgcctgccaccacaccgactaattttgtatttttagtagagacggggtttctcgatgttggtcaggctgctctcgaactcctgacctcagatgatccgcccgcctcggcctcccaaagtgctgggattacaggcatgagccaccacgcccggcccataaatgcttttcttaatcatattaaggaagttcctttctagtcgtagttttctgagtgtttttattatgaaagactttcagatttttgtaaaatgcttttcctgcgttaattgagataatcatatgggttttctccccctttactctattgatgtaatgcattacaacgattttttttaatgtttacccatctttgcattcctggaataaatactagttgattgtgctgtataattcttaaaatatgctgctggatttgttttgttagtatttggttgcattcttttgcatctatattcataagggatattgatctgtaactattattttcttgtggtggctttatctggctttggtatcaagataatgctggccacattggctaagttagaaagtgttctttcttctattttttgaagagcttgaaaagagtcgtgttaattcttctttaaatatttggtacaattcactattaaagccactagtcctgggcttttcttagtttgaaagtttttgattactaattcaatctcttttacacctagattagattttgtattttttccttagccacttttggtaatgtgtgtgcttccgggaatttggccaggtcatctctgttatctaatttgctggcatccaaatgttcataatgttctcttgtaatcctttttattatagaaatgatatacagaaaaggtcagttaccactttcttttatgattgcattaatttgcttcttttctctttttttctctagtcagtcttgctaaaggttggtctgttttgttgatttttttcaaagaaccaacttttgattttgttgattctataatttttctgctctgtattttgtgtatatccattctaatttttattagttcctttattctgttagctgtgagtttagttggctcttcttttttattttcttaaggtggaagactaagttattgagatatatcttgtttttttttttgatgtaggtatttaaagctataaaatttcctctgagcattgcttttgctgcaatttataagtattggtatgttacatattcagttagttttttatatacacatttaagcttttgctaatctaccttgtgatttcttcattgacttattgcttgtttgagtgtgtcaacaatttccacgtatttgtgaatattctagttttccttttgttattgatttctagtttcatttcattgtggtaagaaacaatactttttatgatctcaacagtttaaaatttattaagacttattttctggtctaacatatgatctatcctggaaaatgtatcatgtgcacttgagaaaaatgtatattctgatttttttaggtgggtggcatgttctattaatatatatgtctgttagctctaggtgaattatagtgatgttcaaagcctccattttgttattaatataaccatgccagatttttaatgctgtccatttgcataatatgtctctttccattctttttcttttacatgatcactttatatttatagtatatttcttgcacacagaatgtaattgaaacttacttttctaaaatctactttcttttttttttttttgagatggagtcttgctctgttgcccaggctggagtgcagtggcacaatcttggctcactgcaatctctgcttcccgggttcaagcaattctcctgtctcagcctcccgagtagctgggattacaggcacctgtatttttagtagagacggggtttcaccatgttggtcaggctggtcttgaactcctgacctcgtgatccacctgccttggcctcccaaagtgctgggattacaggcgtgagccactgcacccagcccctaaaatctgctttcatagcatctgtcttgtacttggaacatttagtccatgtgtatttgaatatttattgatatgtttgggtttatgtttacaatcctgctatttgttttctttttgttccatgtgttttttgttttttatttctgcatcttttggaaaaatcagattttttggtaattcatttttcttattttattggctttttagagaaaccttttaatatctttgtgagttttagggattacattatttattcttattattattccatttagaattaatattgttaatcccattcaaaaatttgtttatttcgcttttcagttctagaattttcatgttttcttcagttgttgtttctctgttgttattctccatttgttcattatatctatcttttctttgaaatccttaaaaatatttataatagctattttaaagtcctctgctaatcccaatgtctgggacatcttggcttctgttgatattgaatacttctttttttccccttaattaagggtttcattttcctgctttttcacatatctagtagatttttattatgctctgtgtgctatgaatgagatattataggaagcctggtcaatattgtctctttttaaagggtgttgtatttagttctgccaagaagttaaattaccagatttacttgatgtggctttagtctttgttagagctggtctattcctgctttgttcttacttctcaggtaatgaccttcctgagtttcagctggatgcctgaagtgctcagctgcatttttctactctggctattctgaaatgcaatattttccagacctacccaacctttggtattcatctcccagacctgtggctgcttctctttgctgagcctcacaaaatcttgtcctgtgcatggacagccaaggatccttgggaaatctcatgcagacttctaggtccttcctttgtgtagatttcttctctccagtacgttgacctgcttcagagtcctcatcattgctttcttccttttcagctcagcaatactgctgtgtattgtgtgggcttcatttgcctcagccatacctagaacacaacccaaggcagaaagctggagtggacctgaggctcatcacctgtttccttccacccaccatttaaaagcagcttatttgtgtgaaactttttgttgggttctgtgggggatacgtataggcaaatgacagggagcttataatttagccaaagagaaaatgtctatatgttctgtggaattcagagtattttcctcgacttccaaaattattttcctcgacttccaaaatttggggctactagctagttggtaggaaggaattccagatccatttttcttctagatttttttcagactccatgtttcaaaatctagatggaaggtaagaaggaagcaaggagggcatcacatacgagagagacttataccccaaagtggatcattagcacattatgagataatatggtattattctcagaggcctgccatataaaattccttctaatttattttttaatggtatgtgcctgaaagttttctgtctttcatgatcttgaaagcaaaataagaaccagagtagcttatgagagagttttcctgcttcagcccagaaaaatgttgcttctgtcctagaccctttgatctggttcttagtgccagccttcattcctctagatctgtttattgaatactggctctgttgcagagactttgctagaatctgagagataaaagactcaatgccttcaaaacacctacaatccagacacttaaataaatgcaataatatatggttaaggtcttggatagaggaccatcatagaaacacatagaacagacatttagtccatgctagggagagggaagatgtcaaggaatgcttcctggaggtggcgacatctaagctgagttttaaagtgggtgtaagagtgaaggaactgggaataggcaaaacataccttagagagctgcacctccaaagcatggaaggctagataatcttagaacttcaagtggggttcaatgtgcctgggacttggatttcaggcagagaccatagggcaagttggagaacaggtgggcagggtgagatgaagcagagacttttaataaccatcaaacaggcctgccatccaactgctatcagccattttccctttttgaattttatttttttaatctctagagcaatacatgtttatggtaggaaatgaaaaaaatatatagatgggtaaaatgaagtgaattataccacccacaatcctgctgtaaaacaggttttaacaagttataaatccctccagatcaaagtgctactaaactctacttgctattttataacctttttctgttcagagtattttagaaacgtttttacatgttaattgatattcttttgcattatcctgaataatggtatttctttagttatactatcatgtattaaaacattcatacatggtaaacatatttgaatattagacatttcaattgttttcaatgtttggctattaaaaatagtatttagatgaatgttcttggccatacagttaataacttcactgagtccttgtcaataactttattaaatcccaatcttaagacaacaagcctataagattaacataccaggaataattagaaagcattgcaagactatatgtaaccatctccctgttaatcatccttatttgttgcaacatcctaaatagcgaccttcccacactctgcctcataatatttaatctgtcaaatgtaccatccgcaaagggagtgaactggctcgtgaagaggtggtgaagctggttcaactcagtttaacagacaatgagggcctccgtgtgccagggagggagtgagggaactccacagggagctcattctggggagggatgcccctcattatctgcagtaaatgatattagagaagtatgtgtgagactcttggtgtccagaggaggggcatgcagcacagtatgggggtccatggggttgcagcgatttcttagagacggtgccatgcctgggatgaccaccaagttaggatgctgtaaagagtaatcaaataagtttcaacagggttttggcatgaggtgagcagttaatccaaatgatatctgaaatgctttctaatgctgcaagtgtattcctctgtaaaataatcaactacttaaaagacactctcacttgcattatgtcatttaatcctcacagtgtccctacgggacaatagatgtcattcccacttgtacagagcagtaagtgaggctcacaccggtcatagttagaggagtagggaatctaaacctaagtctcttctactccaaaccccaagttttatcagtatgtcacatcgctactgtgggctgaattccttagcaggggttgtttcctccacctgcaatattcttccttttggtttgttcatcctctagagagagaagggatacgcattcatggagatcctactgttgcaaactctgcattaggagttttacatgaattgtctcatttagtcctcacaacagtcctgtgaggcacaggaaagttagatttacctgcccaaattaaggaatgtggccaactcaagagtgacgaaggccaggtgcggtggctcatgcctgtaatctcagcactttgggaggctgaggtgggaggctgaggatcgcttgagcccaggagtttaagaccagccagggcaacacagcaagatttaatctctactagaaataaaaaagaaattagccaggtgcctgtagtcccagttacttgggaggttgaagtgggacgattgcttgagcctgggaggtagaggctgcagtgagccatgatcccatcacttcactccagtcagggtgacagagtgagacccagtcttaaaacaaaacaaaacaaaaaagagtgatggagcaggaactttatctgccatcagagaccatatatgtctttccttatggcccaggtcatttatcatgtccttgctaaacctcctcctctgagccttgttaaagtcctcaaggttagaagagtggtctataattataaaaaattccattgtatgacttcagctacctgcaaaactgaccgagattaatgcattctttgtttgctttcactctttcattccctgttcagcgcttattctacagaaaggtgcctggtagatttaggacatgactgttcataggcctcaaatgcggtcaaattaggaaagtcccctggtttttggtctcaagaggttaattgctgagtactagcctggactgctcaatggattttatgtttaactgttgtgtttatttgtttgtttgtttttgttttgtgagacatatctttctctgtcacctgggctggaggtcagtggtgtgaacatggctcaccgaagcctccatctcctgggctcaagtgatcctcctgcctcagccttcctagtagctgggactacaggcacacagcaccatgactggttaagttttggattttttggtagaaatggggtctcactctttgcgcaggctagttgtgaactcctggtatcaagggattctcccaatgtgctgggatttcggcctcccaaagtgctgggactgcaggcatgagccatcatgcccagcctctgtttaactgttaacatcatgaggtttcctttcaatgaggaagggaggcctggggaggtgtgatggggaagatggagaagggtaggagacatcataaaactgacagaagtgtggcacgattaagaccctggtttcttagaccacataagcaagtgccactattcttttgatcaacatttgattctctatttccttcttagcatatagatagctgcttcaggtcgtgaaaaaatacatttagtgaaatgaaacatagtagatatgttcaccaagaaactaaaaagaaaagttagccacaaactatcttatttcattgaaatgtttggctgaacccataagaatgttgatgaggccattcttggatgcctgtactgaaatgaaccacgagaggaatatttaggatatgttgaagaggctgtttggcttaatgaacaaaggagtttctgcaggcccagggagtggaatgaaaattggcatgatcattttggagaatatatggagtcaaacagagaatttgaatggagttttcaaagaggatatgttaggggaataaaggctgataaacaacacttgtatttgtaggaaaatagggagagtaaatcaaggaattcaaagagaaagagaaactcacttcaagtaggggagaaaaaacccctataattttcactcttccttgtaaataaaaaggaaacaaatgaaaataagatattagaagtcagtaagaatttatgggagtataaagttggttttatggatgcaaagcccttttcactgctgtacgaaactcctggctgcatgctaacaatggacaactgatttccttgcagctgtattttgctgttttttgctcttggcttggacctagcaccctgggtctgtgggaaaccagaactgtcccagagttctggagggtaggccaaggttagatgctggagtgggttctttaatttattgtactgattcttcttgggaagaaagaagattgcttgttagaattttagctacgagagatgactatgaaacagtaaattaactccaacgacctgagtcattttgaaaactcccagtctcaggataaaaaatataatcctatttagaaattcctggtgtgatcacagatgtagcattggttcttttcatgaaacccgtaaattaaaaagtacataatccaaagtcaattaaatagtaagctattataacaaattcttttatttcattagcttttcaaaatgtggataactacacactcaacccaaggaatctacatttttccactgactgctaaagaccaatggaaataactctagtccccgtagcacctcactgtggggtgacctacctttgaaataatgtattggttctagctgatttttatattgttagtcattaagttaggcttgatgagaaacagatataatctgatttggggattcaagtattatattgcatttctcctcacaactagagataaatttgccatggtttttctcttcataggctcatgccaaagtctggcatctctacaatacttctttccgtcccactcagggaggtcaggtgtccattgccctaagctctcactggatcaatcctcgaagaatgaccgaccacagcatcaaagaatgtcaaaaatctctggactttgtactaggttggtttgccaaacccgtatttattgatggtgactatcccgagagcatgaagaataacctttcatctattctgcctgattttactgaatctgagaaaaagttcatcaaaggaactgctgacttttttgctctttgctttggacccaccttgagttttcaacttttggaccctcacatgaagttccgccaattggaatctcccaacctgaggcaactgctttcctggattgaccttgaatttaaccatcctcaaatatttattgtggaaaatggctggtttgtctcagggaccaccaagagagatgatgccaaatatatgtattacctcaaaaagttcatcatggaaaccttaaaaggtatgattgtgggtaaagttctcatttcctgccaaaatcttctggaaaaaaatctctaagattatctaacataaatgatgtgaatttatatttttaaatcctaatggagacattcattttggcaatagtagaatgcattcatttaacacctttctcatttggagtcttgaggaacttgaattaatttttaaaaacccatttgtaaatgagaaactgggttataatatttgtaattacttaactttcagttattaatctagatttttagattaaattgaacataaaacaaatcccaggatatctagctctctgcacatgtttttcagttcttgttattttggttgaataaaacactttaaagaaaaaggaatgtccatgttttctagagaaaatagtataaatagatcatgcttttaaagccttcatttatttatttattgcatcagacacaaagctgggtgtctaggatggaaagtggtacaagacatctttccagccctgtagaatatctattataaataaggaactattttttcaaggtgctcagaaatccaaaaaacatattagataggccaattttgagggcatttatttgtagagttatataggtttgattagagtctttcgtcaagaagaaaaatcattggcttaccaaacgagaagcattacactttatttatttaagtaggaaacgctcagctgctcttgaaccatgatgcaagtgcccagcgaagggtcatgttgctcttgtcccctcttccctttgcagccatcaagctggatggggtggatgtcatcgggtataccgcatggtccctcatggatggtttcgagtggcacagaggttacagcatcaggcgtggactcttctatgttgactttctaagccaggacaagatgttgttgccaaagtcttcagccttgttctaccaaaagctgatagagaaaaatggcttccctcctttacctgaaaatcagcccctagaagggacatttccctgtgactttgcttggggagttgttgacaactacattcaagtaagtcagctgacaaaaccaatcagcagtctcaccaagccctatcactagtaagtagtgcttccttcctaggctgattgtcatggcacattgtccgttctttgagccaaaaacaattccttatgagtacactaagggcacaatttggaatgctgcacccttctctccaaaactcttccaatcttcatcttgtttaagttagatccaaagataaataaatttaaagcatatcaatatttaagatccgattaagacagtaaaaagataaaacactctcttttcatactgtggtttttgatcctttttaaggcagttgagttttttcatgaacaggatctaacacagaactccaaagcctctgagtttcagtggtgctgctgagactgaggcaggaacattaggcagagtcctccagaggcacaactgtgggctccacaaatgtgcagaaataccctaagaaagtaaaccctagatccaatgattcactggtcagaatgtcttttttagcaatagtcattgaaatgatacgaaatttcttcagaatgatcaaccaatatttattgagcatcttctcagtagtaagcccttaacattctttcagacttcctaaattttgaaggggcttgttttccagcatttgactggatactctagtaagcacttattggatgtctagtgtgtccgaagccttgtgttagttgctcgggtcgcttggttaaggggagtgcaggtagagggtatactgagatgagtaagggtaacctttgctttcaaaggagcaaaggagtctactgagcgaaaacaatgtatgcacaaatgatgcaatggagtgaagcgggcatggtggtaagtaacaagggcggggctgggggattgctgctgatagagtcccaagtgtgaaaatagccctcaagacagagacagagttcagtgtccatagacaagcagttggctttgacatgttgggttatggtagccaattaattggttctgcaaatcacagcttgaaaggaaacacttggaagaatgtgaaatgggttgctgttttcttgtaaatatccaattgaaatcttttatttataaggaaataaattaacaccatccttagtacattttttgctggttgggattattcttctttttcagaccacccagttcattttacaggcagtctcagacttaaaccctcgccttccatttaaaagatgactggctcacgcctgtaatcccagcactttgggaggccgaggcgggcggatcatgaggtcaggagatcaagaccatcctgaataacacggtgaaaccccgtctctactaaaaatacaaaaaaaaaaaaaaaaattatccgggtgtggtggcgggcacctgtagtcccagctactctggaggctggggcaggagaatggcatgaacccaggaggcggagcttgcagtgagccgagattgcgccactgcactccagcctgggtgacagagcaagagtccgtctcttaaaaaaaaaaatgactggatgtgtcatcttttatgccaggatatgtgagcccaggagaaaggcttctgagctccctcctgctcggtgtgcaattttctgccctgccccgactctctccttctctcccagcctcctgctatttgaaatctccttatcctaatttccctcctcagagtggattccactgtggggttcagagaggatctgaggtgggagaagtgaggctggtgaggaagaaggggaggagaaagggaagaagacctccgtagccttccttcctcctcctctttactggggttggggatagatcggatggtccctggtccttgttctatctcttgaccttctgcctgctccctgctgagcacggatctctgatagcagcctgagtctggcaggttcagtcctttgtatgcggcacaatctcccagccagcattgctgtgcagatcatgggaacgaatgcagaacaagagtgggggtgtcggagggagccctacttctcctgttctattcctcatcagggggctgtgcgctggctttgggaattggtaaatagtgagaaagtcttaagggtacatcctatttccttgagggagaagagaaaacgctggtcagaagcaataagtatagcagtgaatagcaagggagatgggagataattccttttcctactacactctagaagctattgttttagaatctgacctaaggtcagccactaattggccccagaggtctctctctcagatcacacggtccttttttcctcatcagcttggggaccccacccctcctcctggcagtctcctcctgtgcagaacccaacaaacacaaaattaagtcactctcaaacccacagcagatgagagcttctctggaagctccctggtggggaaaggctgcaattgctattttcttcttctggttttcacctcaggctttgtgttatattgacagtacccttctcaagctaactccctaactgacctgacgtagtcaaaataagttctttgtatgtcagttctgaggtgtgtgtgttttcacttacaaacagtactctacagctttaagacattatattaaagtcctgagaagtgatttttaaaccactgaacttcatcttttccctcctggctagtatttcagactttcagtgtttgaggcatgcatttcacctgaacaacttgaaaaataatatcctaagaagcacacaacctgactttaggctcattcacatggattgtcactttacttggacccactttctcggctgagaggtttgttttcccataaccacggatgctcatagttaatataaatattgaactcactatgtagtgaggacatagagcctctttaacattggtccctgttaggagaaagtttctcccataacatactaaatacatgttttaatagccgttccttctgaaaggtccaacttcactattttatttttttagtaaaatcttagttaacaaattaatggaggttaggtggaattttgccccaaaagtcctgtattttctttttttttttttctttttttttgacagagtcttgctctgtcgcccaggctggagtgcagtggcgtgatctcggctcactgcaagctccgcctcctaaggtcacgccattctcctgcctcagcctcctgagtagctggggctacaggtgcccgccaccgcacccggctaattttttgtatttttagtagagaccgggtttcactgtgttagccaggatggtctcaatctcctgacctcgggatccacccacctcggcctcccaaagtgctgggattacaggcgtgagccaccatgcccggcctgtcctgtattttcaagaaactttttttttcctccagaaatgataccctagtctttcatatttgttttcagatggactgaataaaagctgttgttttggaacaatcacggttaaaaaaaaaagttatgaatttagtcaactcagagctctataaaaataatccaaaaaattccttcaaactctgaacgcttcaaaagagcgtgcaaatattctgtccttcaaagctaaggaaacatgatttgtggggtgcatcacagtggaaaaatactctgacagcattcccacagcattaggggaagtgcatgtgtgggtgttctgcaagggacaattctccagaaaaggcaatttccctttgacatgctgtttttaatgacttttctttataaacacacttatctctccagagaaatagcagtgcatttgcaacaggcccgtaaaatgcaacaaaacctctgctatggtttctgacccctgcttttatacagagcatcagaccaaggaacctgttctaacaggattatttcagaggggaacacaggcttagggtgcagatcttccagctggatttttcactttgcattccctccacagcagacacatgaaggaatgattttgtgattttgattttataatttgcacacttttcctaaatactttttttaaatttttatttgggaggattttatagcatatgattgagaactataatcatcatcattgttacagaagaataatttagaaaaattttttaactacgttaaaaattccactatgggtggatgacaatattgttctttccttccacattctccctccttagactttcttttctttttttctattttttttttgagatgaagtctcgctctgtcactcaggctggagtgcagtgccatgatcctggctcactgcaacctctgcctcccgggttcaagtgattctcccgcctcagcttcctgagtagctgggattacaggtgtgcaccaccacacctggctactttttgtatttttagtagagatggggtttcaccatgttggtcaggctggtctcaaactcctgatctcatgatctgcccgccttggccccgcaaagtgccgggattacaggcgtgagccactgcgcctggcctctctctcggactttctaccatcagtcagattgaatttgttaaattctgtcactgaccctaaacccaacaaaaggcaagagttatgtttatttagcacttcctctacctatagcaaacctcaatttagagcgtaattttaagcacaatttaattataaatatcttttcattttcttacttaactcactcagttttttaaatcttcttttttgagacaagatcttgctctgtcactgaggccgatgtacagtgatgtgatcatgacttactgcagccttgacctcccaggcttaggtgatcctcatacctcagcctcccgagcaactaggactacaggcccgtgccaccatgccgggccaagacggggtttggacgtgttgccccagctggtctccaactcctggcctcaagtgaccctcccgcctcggcctctcaaagtgctgggattataggcatgagccaccgcacctggccaactcactcacattttaagttttttctttttttcatctagttttttttctttttaaatttgaaagcctcatgacattaatgatttcttacattaaaagaaaaacacccaaaaatactctgcttacataacaccgacaagtagtgtgcaagactcattagcatttgtcatctgaagtgaccaaatccagacttttgggggtcacattaaagaaacagttgaagagttagaactatgggtaaagcgagtgtgcatatcagaaagtggaatattgtcttcctcaggagctgacaatttatgaaaaatagttcacattctcagctagaaaggcttctatttttgctcatattcctggctagttttgctgaaataattgctttgaattacttcctcaggactgcccaggtgacgctaatgtttactctgcccttcacaggtagataccactctgtctcagtttaccgacctgaatgtttacctgtgggatgtccaccacagtaaaaggcttattaaagtggatggggttgtgaccaagaagaggaaatcctactgtgttgactttgctgccatccagccccagatcgctttactccaggaaatgcacgttacacattttcgcttctccctggactgggccctgattctccctctgggtaaccagtcccaggtgaaccacaccatcctgcagtactatcgctgcatggccagcgagcttgtccgtgtcaacatcaccccagtggtggccctgtggcagcctatggccccgaaccaaggactgccgcgcctcctggccaggcagggcgcctgggagaacccctacactgccctggcctttgcagagtatgcccgactgtgctttcaagagctcggccatcacgtcaagctttggataacgatgaatgagccgtatacaaggaatatgacatacagtgctggccacaaccttctgaaggcccatgccctggcttggcatgtgtacaatgaaaagtttaggcatgctcagaatgggaaaatatccatagccttgcaggctgattggatagaacctgcctgccctttctcccaaaaggacaaagaggtggctgagagagttttggaatttgacattggctggctggctgagcccattttcggctctggagattatccatgggtgatgagggactggctgaaccaaagaaacaattttcttcttccttatttcactgaagatgaaaaaaagctaatccagggtacctttgactttttggctttaagccattataccaccatccttgtagactcagaaaaagaagatccaataaaatacaatgattacctagaagtgcaagaaatgaccgacatcacgtggctcaactcccccagtcaggtggcggtagtgccctgggggttgcgcaaagtgctgaactggctgaagttcaagtacggagacctccccatgtacataatatccaatggaatcgatgacgggctgcatgctgaggacgaccagctgagggtgtattatatgcagaattacataaacgaagctctcaaaggtaaggagccctagctgcggctatctcctgaaggttatgtcaccagagggcatgacacttgattaaatctccaacatcaacacacactgccacccttggaatggagggctatccattttgtgcctcactgaaacagtccaagagatatctagcatttccccaaggataaaggagtgtagctaaaagtagaagaccagaaatccctagcccctactctggatctatgcaagcctagattcttgtcttccatcttggatggctccacagcagtcttaactgtttcatgtacataaagcagtacataaagatttaaccttgctgggcatggtggctcacacctgtaatcccagcattttggaaggccaaggcaggaggattgcttgagcctagaagtttgagaccagcctgggcaacatagtgagaccttgtctctactaaaaatcacaaaaattagctgggcacggtggcatatacgcctgcagattcagttacttgggaggagaggcgggaggattgcttgagcttgggaggtccagctgcagtgaatcatgatcacagcactgcaatctggcctgggtgacagagcaagacactatttcaaaaaaaaaaagaccaagcatggtggctcatgcctgtaatcccagcactttgggaggctgaggcaggtggatcatctgaggtcagaagttcaagaccagcctgaccaacatggtgaaaccccgtctctactgaaaatacgaaaattatccaggtgtagtgatgcacacctgtaatctcagctactcgggaggctgaggcagaagaatcacttgaactggggacgtggaggctgcagtgagccaagattgcaccattgcactccagcctgggtgacagagcaagactccatctcaaaaaaaaaaaaaaaaaaaaaaggatttaacccaagtatatcatagtagattgaattatgtaaaacacccatttaacaaccaggtccaggtttgttctctctgtgtagtaaatcaatcactgtgacacaggttttgcaaaagagaaaagatttatttgtaaggggaccaagcgagggggtgggagaataacttccaatcctgcctctctgaagacaaggcttaggaatatgtatgggttagggaatgggtggtctaaggcatggtgaagagtgattggcagggggggaaaatgaagtaacaggttagacacatgcacagaaaatggtggtgttagcatgatctgagggcagagttttgggccctctgacgtcaaaagaccacctctcaggcacttgtgcaggcccagtggaagggtcagtggtcttaactagtttgaactggacaggagctgccccaagttcttggaaaaacaactgaagtgaccattgccatggtaacctatgaatgtcatcagtaaagtagccagtgaaggttaagtttcagcatacaatgggacaaccttcagcttcatggaaaaaggaaaaaaaaaaaacacatacacacacgaaaagcaagtgaccaaaagcaagcaggacaggcagacctgatccaattaacccctgggtttcaaccctgctaaatgcagctcaatatttgtcttgataatttgcctatttggctttacataaaataaagccttttctgatgaaatctaattgagtctgaagttgtattaaatggtatcggaaacttcccagcaggaaggctacgtaaaagtggccgggcgtggtgactcacgcctgtaatcccagcactttgggaggctgaggcaggcagatcacaaggtcaagaaatcgagaccatcctggccaacatggcgaaatcccatctctactaaaaaaaaaaatacaaaaatttgccaggtgtggtggtgctcacctgtagtcccagctactcaggaggctgaggcaggagaatctgttgaacctgggaggcggaggttgcagtgagtcaagatggtgccattgcactccagcctgtgtgacagagcaagactccgtctcaaaaaaaaaaaaaagtgatgtgttgtgtgcaaaatacgtaataactactctcctatccttttgtttttccagcccacatactggatggtatcaatctttgcggatactttgcttattcgtttaacgaccgcacagctccgaggtttggcctctatcgttatgctgcagatcagtttgagcccaaggcatccatgaaacattacaggaaaattattgacagcaatggtttcccgggcccagaaactctggaaagattttgtccagaagaattcaccgtgtgtactgagtgcagtttttttcacacccgaaagtctttactggctttcatagcttttctattttttgcttctattatttctctctcccttatattttactactcgaagaaaggcagaagaagttacaaatagttctgaacatttttctattcattcattttgaaataattatgcagacacatcagctgttaaccatttgcacctctaagtgttgtgaaactgtaaatttcatacatttgacttctagaaaacatttttgtggcttatgacagaggttttgaaatgggcataggtgatcgtaaaatattgaataatgcgaatagtgcctgaatttgttctctttttgggtgattaaaaaactgacaggcactataatttctgtaacacactaacaaaagcatgaaaaataggaaccacaccaatgcaacatttgtgcagaaatttgaatgacaagattaggaatattttcttctgcacccacttctaaatttaatgtttttctggaagtagtaattgcaagagttcgaatagaaagttatgtaccaagtaaccatttctcagctgccataataatgcctagtggcttcccctctgtcaaatctagtttcctatggaaaagaagatggcagatacaggagagacgacagagggtcctaggctggaatgttcctttcgaaagcaatgcttctatcaaatactagtattaatttatgtatctggttaatgacatacttggagagcaaattatggaaatgtgtattttatatgatttttgaggtcctgtctaaaccctgtgtccctgagggatctgtctcactggcatcttgttgagggccttgcacataggaaacttttgataagtatctgcggaaaaacaaacatgaatcctgtgatattgggctcttcaggaagcataaagcaattgtgaaatacagtataccgcagtggctctaggtggaggaaaggaggaaaaagtgcttattatgtgcaacattatgattaatctgattatacaccatttttgagcagatcttggaatgaatgacatgacctttccctagagaataaggatgaaataatcactcattctatgaacagtgacactactttctattctttagctgtactgtaatttctttgagttgatagttttacaaattcttaataggttcaaaagcaatctggtctgaataacactggatttgtttctgtgatctctgaggtctattttatgtttttgctgctacttctgtggaagtagctttgaactagttttactttgaactttcacgctgaaacatgctagtgatatctagaaagggctaattaggtctcatcctttaatgccccttaaataagtcttgctgattttcagacagggaagtctctctattacactggagctgttttatagataagtcaatattgtatcaggcaagataaaccaatgtcataacaggcattgccaacctcactgacacagggtcatagtgtataataatatactgtactatataatatatcatctttagaggtatgattttttcatgaaagataagcttttggtaatattcattttaaagtggacttattaaaattggatgctagagaatcaagtttattttatgtatatatttttctgattataagagtaatatatgttcattgtaaaaatttttaaaacacagaaactatatgcaaagaaaaaataaaaattatctataatctcagaacccagaaatagccactattaacatttcctacgtattttattttacatagatcatattgtatatagttagtatctttattaatttttattatgaaactttcctttgtcattattagtcttcaaaagcatgatttttaatagttgttgagtattccaccacaggaatgtatcacaacttaaccgttcccgtttgttagactagtttcttattaatgttgatgaatgttgtttaaaaataattttgttgctacatttactttaatttccttgactgtaaagagaagtaattttgctccttgataaagtattatattaataataaatctgcctgcaactttttgccttctttcataatca

SEQ ID NO: 4 - DNA, 250 bp, Homo sapiens
ccgacgtccgcgggcgacgcctgccgcaccttgcccgctgccgcgcccctcccgggcacccctcgccctcggcgcccctgcccccgcccccagtgccagggcggaggcagtcccggctcgcaggtaattattgccagcggagcccgccggggagcgggggtgggcgcgccggcggtgggcgggcgggcgcggcggggcgcgggcataaaggggcgcggcgcggggccccggagcctggctcccgcgcagc

(19) United States
(12) Patent Application Publication
(10) Pub. No.: US 2022/0008520 A1
(43) Pub. Date: Jan. 13, 2022

(54) METHODS AND MATERIALS FOR REDUCING AGE-RELATED STRIATED MUSCLE AND COGNITIVE DECLINE

(71) Applicant: University of Pittsburgh - Of the Commonwealth System of Higher Education, Pittsburgh, PA (US)

(72) Inventors: Fabrisia Ambrosio, Pittsburgh, PA (US); Aaron Barchowsky, Pittsburgh, PA (US); Amin Cheikhi, Pittsburgh, PA (US); Rada Koldamova, Bethel Park, PA (US); Iliya Lefterov, Bethel Park, PA (US); Amrita Sahu, Pittsburgh, PA (US)

(21) Appl. No.: 17/294,726

(22) PCT Filed: Nov. 21, 2019
(86) PCT No.: PCT/US2019/062682

§ 371 Date: May 18, 2021

Related U.S. Application Data
(60) Provisional application No. 62/770,615, filed on Nov. 21, 2018.

(51) Int. Cl. A61K 38/47; A61K 9/127; A61P 21/06

(52) U.S. Cl. CPC A61K 38/47; A61K 48/00; A61P 21/06; A61K 9/127

(57) ABSTRACT

This document provides methods and materials for treating aging. For example, a mammal having, or at risk for developing, an age-related impairment (e.g., age-related cognitive decline) can be treated by increasing the level of one or more myokine polypeptides (e.g., one or more Klotho polypeptides) within cells within the mammal. This document also provides methods and materials for increasing the ability of muscle progenitor cells to regenerate muscle cells by increasing the level(s) of one or more myokine polypeptides (e.g., an α-Klotho polypeptide) within a muscle progenitor cell.

### CROSS-REFERENCE To RELATED APPLICATIONS

This application claims the benefit of U.S. Patent Application Ser. No. 62/770,615, filed on Nov. 21, 2018. The disclosure of the prior application is considered part of (and is incorporated by reference in) the disclosure of this application.

### STATEMENT AS To FEDERALLY SPONSORED RESEARCH

This invention was made with government support under AG052978 awarded by National Institutes of Health. The government has certain rights in the invention.

### BACKGROUND

### 1. Technical Field

This document relates to methods and materials for treating aging. For example, a mammal having, or at risk for developing, an age-related impairment (e.g., sarcopenia and/or age-related cognitive decline) can be treated by increasing the level of one or more myokine polypeptides (e.g., one or more Klotho polypeptides) within cells within the mammal. This document also relates to methods and materials for increasing the ability of stem cells to regenerate tissue-specific cells (e.g., increasing the ability of muscle progenitor cells (MPCs) to regenerate muscle cells). For example, the ability of MPCs to regenerate muscle cells can be increased by increasing the level of one or more myokine polypeptides (e.g., an α-Klotho polypeptide) within a MPC.

### 2. Background Information

Aging is associated with a loss of muscle mass (sarcopenia) and an impaired skeletal muscle regenerative capacity after an acute injury, resulting in declines in force-producing capacity. The impaired regenerative response of aged muscle is characterized by a shift from functional myofiber repair following injury to fibrotic deposition (Brack et al., Science, 317(5839):807-10 (2007)). This increased fibrosis has been attributed to muscle stem (satellite) cell (MuSCs) dysfunction (Brack et al., Science, 317(5839):807-10 (2007)). Aging also is generally associated with cognitive dysfunction. Brain atrophy begins in the third decade of life, and the rate of decline increases steadily over time (Kirk-Sanchez et al., Clin. Interv. Aging, 9:51-62 (2014); and Fjell et al., J. Neurosci., 29(48):15223-31 (2009)).

### SUMMARY

This document provides methods and materials for treating aging. For example, a mammal having, or at risk for developing, an age-related impairment (e.g., sarcopenia and/or age-related cognitive decline) can be treated by increasing the level of one or more myokine polypeptides (e.g., one or more Klotho polypeptides) in cells (e.g., neurons, stem cells such as muscle stem cells, or muscle cells) within the mammal. In some cases, one or more myokine polypeptides (e.g., one or more Klotho polypeptides) can be administered to a mammal having, or at risk for developing, an age-related impairment (e.g., sarcopenia and/or age-related cognitive decline) to treat the mammal. In some cases, nucleic acid encoding one or more myokine polypeptides (e.g., one or more Klotho polypeptides) can be administered to a mammal having, or at risk for developing, an age-related impairment (e.g., sarcopenia and/or age-related cognitive decline) to treat the mammal. In some cases, an exosome containing (a) one or more myokine polypeptides (e.g., one or more Klotho polypeptides) and/or (b) nucleic acid (e.g., mRNA) encoding one or more myokine polypeptides (e.g., one or more Klotho polypeptides) can be administered to a mammal having, or at risk for developing, an age-related impairment (e.g., sarcopenia and/or age-related cognitive decline) to treat the mammal. In some cases, a gene editing system designed to reduce or eliminate methylation of a promoter that directs expression of a myokine polypeptide (e.g., a Klotho polypeptide) can be administered to a mammal having, or at risk for developing, an age-related impairment (e.g., sarcopenia and/or age-related cognitive decline) to treat the mammal.

This document also provides methods and materials for increasing the ability of stem cells (e.g., MPCs) to regenerate more differentiated cells (e.g., tissue- or organ-specific cells such as muscle cells). For example, the ability of MPCs to regenerate muscle cells can be increased by increasing the level of one or more myokine polypeptides (e.g., an α-Klotho polypeptide) within a muscle progenitor cell. The levels of one or more myokine polypeptides (e.g., an α-Klotho polypeptide) within stem cells (e.g., MPCs) can be increased by administering one or more myokine polypeptides (e.g., an α-Klotho polypeptide) and/or by administering nucleic acid encoding one or more myokine polypeptides (e.g., an α-Klotho polypeptide) to a mammal having stem cells (e.g., MPCs) as described herein. In some cases, the levels of one or more myokine polypeptides (e.g., an α-Klotho polypeptide) within stem cells (e.g., MPCs) can be increased by administering an exosome containing (a) one or more myokine polypeptides (e.g., one or more Klotho polypeptides) and/or (b) nucleic acid (e.g., mRNA) encoding one or more myokine polypeptides (e.g., one or more Klotho polypeptides) to a mammal having stem cells (e.g., MPCs) as described herein. In some cases, a gene editing system designed to reduce or eliminate methylation of a promoter that directs expression of a myokine polypeptide (e.g., a Klotho polypeptide) can be used to increase the level of one or more myokine polypeptides (e.g., an α-Klotho polypeptide) within stem cells (e.g., MPCs).

As demonstrated herein, young skeletal muscle displays a robust increase in local α-Klotho polypeptide expression following an acute muscle injury with transient demethylation of the Klotho promoter. However, aged muscle displays no change in Klotho promoter methylation and no increase in α-Klotho polypeptide expression following injury. Levels of α-Klotho polypeptides in MPCs derived from aged mice are decreased relative to those levels in young animals, and genetic knockdown of α-Klotho polypeptide expression in young MPCs confers an aged phenotype with pathogenic mitochondrial ultrastructure, decreased mitochondrial bioenergetics, mitochondrial DNA damage, and increased senescence. Further supporting a role for α-Klotho polypeptides in skeletal muscle vitality, mice heterozygously deficient for Klotho (Kl+/−) have impaired MPC bioenergetics that is consistent with a defective regenerative response following injury. Indeed, the regenerative defect of Kl+/− mice mice is rescued at the cellular and organismal level when mitochondrial ultrastructure is restored through treatment with the mitochondria-targeted peptide, SS-31. Also, as demonstrated herein, systemic delivery of exogenous α-Klotho polypeptides rejuvenates MPC bioenergetics and enhances functional myofiber regeneration in aged animals in a temporally-dependent manner. Together, these findings demonstrate a role for α-Klotho in the regulation of MPC mitochondrial function and skeletal muscle regenerative capacity.

The ability to promote muscle vitality and regeneration provides an opportunity to treat mammals having, or at risk for developing, an age-related impairment (e.g., age-related cognitive decline) and/or to treat mammal having acute muscle injury.

In general, one aspect of this document features a method for reducing sarcopenia or age-related cognitive decline within a mammal. The method comprises, or consists essentially of, (a) identifying the mammal as having sarcopenia or age-related cognitive decline, and (b) administering an α-Klotho polypeptide or a nucleic acid encoding the α-Klotho polypeptide to the mammal. The mammal can be a human. The method can comprise administering the α-Klotho polypeptide to the mammal. The method can comprise administering the nucleic acid to the mammal. The nucleic acid can be a viral vector. The viral vector can be an AAV8 vector.

In another aspect, this document features a method for reducing sarcopenia or age-related cognitive decline within a mammal. The method comprises, or consists essentially of, altering a promoter nucleic acid sequence of an α-Klotho polypeptide present within a neuronal cell to remove one or more methylation sites. The mammal can be a human. The altering can occur in vivo. A gene editing system can be used to alter the promoter nucleic acid sequence. The gene editing system can be a TALEN system or a CRISPR/Cas9 system.

In another aspect, this document features a method for reducing sarcopenia or age-related cognitive decline within a mammal. The method comprises, or consists essentially of, administering exosomes comprising an α-Klotho polypeptide or a nucleic acid encoding the α-Klotho polypeptide to the mammal. The mammal can be a human. The method can comprise administering exosomes comprising the α-Klotho polypeptide to the mammal. The method can comprise administering exosomes comprising the nucleic acid to the mammal.

In another aspect, this document features a method for increasing the ability of muscle progenitor cells to regenerate muscle cells within a mammal having a muscle impairment. The method comprises, or consists essentially of, (a) identifying the mammal as having the muscle impairment, and (b) administering an α-Klotho polypeptide or a nucleic acid encoding the α-Klotho polypeptide to the mammal. The mammal can be a human. The muscle impairment can be sarcopenia. The method can comprise administering the α-Klotho polypeptide to the mammal. The method can comprise administering the nucleic acid to the mammal. The nucleic acid can be a viral vector. The viral vector can be an AAV8 vector.

In another aspect, this document features a method for increasing the ability of muscle progenitor cells to regenerate muscle cells within a mammal. The method comprises, or consists essentially of, (a) identifying the mammal as being in need of muscle progenitor cells having an increased ability to regenerate muscle cells, and (b) administering an α-Klotho polypeptide or a nucleic acid encoding the α-Klotho polypeptide to the mammal. The mammal can be a human. The method can comprise administering the α-Klotho polypeptide to the mammal. The method can comprise administering the nucleic acid to the mammal. The nucleic acid can be a viral vector. The viral vector can be an AAV8 vector. In another aspect, this document features a method for increasing the ability of muscle progenitor cells to regenerate muscle cells. The method comprises, or consists essentially of, altering a promoter nucleic acid sequence of an α-Klotho polypeptide present within a muscle progenitor cell to remove one or more methylation sites. The muscle progenitor cell can be a human muscle progenitor cell. The altering can occur in vitro. The altering can occur in vivo. A gene editing system can be used to alter the promoter nucleic acid sequence. The gene editing system can be a TALEN system or a CRISPR/Cas9 system.

In another aspect, this document features a method for increasing the ability of muscle progenitor cells to regenerate muscle cells within a mammal having a muscle impairment. The method comprises, or consists essentially of, administering an exosome comprising an α-Klotho polypeptide or a nucleic acid encoding the α-Klotho polypeptide to the mammal. The mammal can be a human. The muscle impairment can be sarcopenia. The method can comprise administering an exosome comprising the α-Klotho polypeptide to the mammal. The method can comprise administering an exosome comprising the nucleic acid to the mammal.

In another aspect, this document features a method for increasing the ability of muscle progenitor cells to regenerate muscle cells within a mammal. The method comprises, or consists essentially of, administering an exosome comprising an α-Klotho polypeptide or a nucleic acid encoding the α-Klotho polypeptide to the mammal. The mammal can be a human. The method can comprise administering an exosome comprising the α-Klotho polypeptide to the mammal. The method can comprise administering an exosome comprising the nucleic acid to the mammal.

In another aspect, this document features a method for increasing the ability of a stem cell to regenerate a more differentiated cell within a mammal. The method comprises, or consists essentially of, administering an α-Klotho polypeptide, a nucleic acid encoding the α-Klotho polypeptide, or an exosome comprising the polypeptide or the nucleic acid to the mammal. The mammal can be a human. The method can comprise administering the α-Klotho polypeptide to the mammal. The method can comprise administering the nucleic acid to the mammal. The nucleic acid can be a viral vector. The viral vector can be an AAV8 vector. The method can comprise administering the exosome to the mammal. The stem cell can be a muscle progenitor cell. The stem cell can be an aged stem cell. The stem cell can be present within a human over the age of 50.

In another aspect, this document features a method for reducing sarcopenia or age-related cognitive decline within a mammal. The method comprises, or consists essentially of, administering vesicles comprising an α-Klotho polypeptide or a nucleic acid encoding the α-Klotho polypeptide to the mammal. The mammal can be a human. The method can comprise administering vesicles comprising the α-Klotho polypeptide to the mammal. The method can comprise administering vesicles comprising the nucleic acid to the mammal.

In another aspect, this document features a method for increasing the ability of muscle progenitor cells to regenerate muscle cells within a mammal having a muscle impairment. The method comprises, or consists essentially of, administering a vesicle comprising an α-Klotho polypeptide or a nucleic acid encoding the α-Klotho polypeptide to the mammal. The mammal can be a human. The muscle impairment can be sarcopenia. The method can comprise administering a vesicle comprising the α-Klotho polypeptide to the mammal. The method can comprise administering a vesicle comprising the nucleic acid to the mammal.

In another aspect, this document features a method for increasing the ability of muscle progenitor cells to regenerate muscle cells within a mammal. The method comprises, or consists essentially of, administering a vesicle comprising an α-Klotho polypeptide or a nucleic acid encoding the α-Klotho polypeptide to the mammal. The mammal can be a human. The muscle impairment can be sarcopenia. The method can comprise administering a vesicle comprising the α-Klotho polypeptide to the mammal. The method can comprise administering a vesicle comprising the nucleic acid to the mammal.

In another aspect, this document features a method for increasing the ability of a stem cell to regenerate a more differentiated cell within a mammal. The method comprises, or consists essentially of, administering a vesicle comprising an α-Klotho polypeptide or a nucleic acid encoding the α-Klotho polypeptide to the mammal. The mammal can be a human. The method can comprise administering a vesicle comprising the α-Klotho polypeptide to the mammal. The method can comprise administering a vesicle comprising the nucleic acid to the mammal. The stem cell can be a muscle progenitor cell. The stem cell can be an aged stem cell. The stem cell can be present within a human over the age of 50.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 shows that α-Klotho expression is increased in young skeletal muscle after injury, but the response is attenuated with age. A-D. Immunofluorescent imaging of α-Klotho (green) and F-actin (red) in uninjured skeletal muscle from uninjured young (UIY; 4-6 months) and old mice (UIO; 22-24 months) as well as 14 days post injury (dpi) in young (YI) and old (OI) mice (n=3/group; scale=50 μm; ****p<0.0001). E. Quantification of α-Klotho across the four comparison groups, UIY, UOI, YI, and OI. F. ELISA analysis of serum obtained from UIY, UOI, YI and OI mice 14 dpi. (n=8-11/group). G. RT-PCR analysis of α-Klotho in young and old muscles a 0 (control), 3, 7, and 14 dpi (n=3-4/age/time-point) H. MSPCR analysis of young and old muscle at 0 (control), 3, 7 and 14 dpi. (n=3-4/age/time point). I. ChIP analysis of DNMT3a in young and old muscle at 0 (control), 3, 7 and 14 dpi (n=3-4/age/time point). J. ChIP analysis of H3K9me2 in the Klotho promoter in young and old muscle at 0 (control), 3, 7 and 14 dpi (n=4/age/time point). *p<0.05 compared to young, sex-matched uninjured muscles #p<0.05 indicates a significant difference between young and aged groups at the respective time-point.

FIG. 2 shows that genetic and muscle-specific loss of α-Klotho impairs skeletal muscle regeneration (A) Immunofluorescence of α-Klotho (green) and laminin (red), as well as F-actin (red) and Sirius red stain and in wild type and Kl+/− mice14 dpi. Scale: 50 μm. (B) Quantitation of α-Klotho (green) in wild type versus Kl+/− mice 14 dpi. (C, D, E) Quantitation of the regenerative index (calculated as % of centrally nucleated fibers), fiber cross-sectional area and collagen deposition. (F) Representative Hematoxylin & Eosin stain of non-targeting control (NTC) and shRNA to α-Klotho (0.2-3.82×106 TU/TA) Scale: 50 μm. (G, H) Quantification of the % centrally nucleated fibers and ratio of myofiber area to total area, respectively, in NTC and Klotho shRNA-treated mice at 14 dpi. (I) Representative immunofluorescence imaging of lipid (red) in NTC and Klotho shRNA treated muscle at 14 dpi. Scale: 50 μm. (J) Quantification of lipid in NTC and Klotho shRNA treated muscle 14 dpi. (K) Quantification of collagen deposition based on Sirius red staining. (L) Quantification of fiber cross-sectional area of regenerating muscle fibers in NTC and Klotho shRNA treated muscle. (M) Representative second harmonic generation (SHG) images of tibialis anterior (TA) muscles injected with NTC or α-Klotho shRNA. Scale: 30 μm. (N) SHG quantification of the regeneration index in NTC and shRNA to Klotho-treated mice at 14 dpi. (O) SHG quantification of myofiber volume in NTC and Klotho shRNA treated muscle 14 dpi. (P) Hang impulse (calculated as hanging time x mouse weight) at 14 dpi represented as a fold-change from baseline score pre-injury. *p<0.05, ****p<0.0001.

FIG. 3 shows that α-Klotho expression in quiescent and activated MuSCs (A) Klotho expression in isolated MuSCs versus whole skeletal muscle lysates as per RNA seq analysis. (B) Representative structured illuminescent microscopy of α-Klotho (green) in young and old MPCs. Scale: 5 μm. (C) Quantification of α-Klotho in young and old MPCs. (D) ELISA analysis of α-Klotho in culture media alone, 48-hour conditioned media from young MPCs and conditioned media of old MPCs. (E) Immunofluorescent co-localization of MyoD (red), phalloidin (white) and α-Klotho (green) 3 dpi. Scale: 50 μm (F) Heat-map representation of α-Klotho expression as well as markers of MuSC activation (MyoD1, Fos, Jun, Myf5) in quiescent and activated cells from RNASeq analysis described elsewhere (van Velthoven et al., 2017 Cell Rep, 21:1994-2004). (G) Immunofluorescence of freshly sorted MuSCs and fibroadipogenic progenitors (FAPs) fixed and stained for α-Klotho (green) and DAPI (blue) immediately after isolation (Day 0) or after activation in culture (Day 3). Scale: 12.5 μm. (H). Quantification of α-Klotho expression in MuSCs and FAPs at Day 0 and Day 3. (I). Quantification of α-Klotho expression in the conditioned media of MuSCs and FAPs freshly sorted from uninjured muscle. Conditioned media was obtained after 3 days in culture. (J) Quantification of α-Klotho (green) expression in MuSCs and FAPs isolated from uninjured muscle and muscle 3 dpi. (K) Immunofluorescence imaging-based quantification of α-Klotho (green) and DAPI (blue) in MuSCs and FAPs freshly sorted from uninjured muscle and muscle 3 dpi. Scale: 50 μm. (L) Immunofluorescent staining of Pax7 (green) and MyoD (red) in MPCs isolated from wild type and Kl+/− mice. DAPI stain in blue. Scale: 50 μm. (M) Quantification of the % of MyoD+ cells in MPCs from wild type and Kl+/− mice. (N) Quantification of the % of Pax7+ cells in MPCs from wild type and Kl+/− mice. (O) Immunofluorescent staining of MyoD (red) and DAPI (blue) in the injured muscles of non-targeting control (NTC) and shRNA to α-Klotho 14 dpi. Scale: 25 μm (P) Quantification of the percentage of MyoD+ nuclei within the injured muscles of non-targeting control (NTC) and shRNA to α-Klotho 14 dpi. *p<0.05, **p<0.01, ***p<0.001, ****p<0.0001).

FIG. 4 shows that loss of α-Klotho drives mitochondrial dysfunction and disrupts mitochondrial DNA integrity (A) TEM images of young, old, young+scramble and young+siRNA MPCs showing mitochondria (M), lipid droplet accumulation (L), as well as endoplasmic reticuli (ER). Scale: 400 nm. (B, C) Seahorse analysis of young, old, young+scramble and young+siRNA MPCs quantifying the basal oxygen consumption rate (OCR) (D) Seahorse analysis of basal OCR on MPCs isolated from wild-type and Kl+/− mice. (E, F) Seahorse analysis of reserve capacity (calculated as the difference between basal and maximum OCR) of young, old, young+scramble and young+siRNA MPCs. (G) Seahorse analysis of reserve capacity of MPCs isolated from wild-type and Kl+/− mice. (H, I) RT-PCR based analysis of mtDNA damage in young, old, young+scramble and young+siRNA MPCs.

(J) RT-PCR analysis of mtDNA damage in MPCs isolated from wild-type and Kl+/− mice. (*p<0.05, **p<0.01, ****p<0.0001).

FIG. 5 shows that mitochondrial structure and function are impaired in Kl+/− mice, but the defect is rescued with SS-31 treatment (A, B) Representative TEM images and analysis of damaged mitochondria of wild-type (WT), Kl+/− and Kl+/−+ SS-31 groups. Scale: 500 nm. (C, D) Representative immunofluorescent images and quantification of cardiolipin content, by Nonyl Acridine Orange staining (NAO, Red) in WT, Kl+/− and Kl+/−+SS-31 MPCs. Scale: 50 μm. (E, F) Representative immunofluorescent images and quantification of ROS as determined by MitoSox staining (Green) on live cells from WT, Kl+/− and Kl+/−+ SS-31 group. Scale: 50 μm. (G) RT-PCR-based analysis of mtDNA damage on WT, Kl+/− and Kl+/−+ SS-31 MPCs. (H, I) Seahorse analysis of basal OCR and reserve capacity of WT, Kl+/− and Kl+/−+ SS-31 MPCs. (J, K) Quantification of MyoD+ cells at the site of injury of TA muscles from WT, Kl+/− and Kl+/−+ SS-31 groups. Scale: 25 μm. (L, M) Representative SHG images and analysis of the percentage of centrally nucleated fibers from WT, Kl+/− and Kl+/−+ SS-31 mice at 14 dpi. Scale: 50 μm. (N) Quantification of myofiber cross-sectional area from WT, Kl+/− and Kl+/−+ SS-31 groups. (O) Hang impulse (calculated as hanging time x mouse weight) at 14 dpi, represented as a fold-change from 1 dpi score during the wire hang test. Each mouse was used as its own control in order to account for baseline variability (shown in FIG. 16). (****p<0.0001, ***p<0.001, **p<0.01, *p<0.05).

FIG. 6 shows that supplementation α-Klotho in vitro improves the mitochondrial function of old MPCs in vitro and improves muscle function in vivo. (A) RT-PCR-based analysis of mtDNA damage in old and MPCs and in old MPCs that received supplementation with recombinant α-Klotho in the culture medium for 48 hours. (B, C) Seahorse analysis of basal OCR and reserve capacity of old and old+Klotho MPCs (D, E) Representative immunofluorescent images and quantification of α-Klotho expression in old muscle 14 dpi after systemic supplementation of α-Klotho via an osmotic pump, as compared to saline-infused control muscles. Scale: 50 μm. (F) Quantification of the percentage of centrally nucleated fibers as per histological analysis across saline and α-Klotho infused animals 14 dpi. (G, H) Representative SHG imaging and quantification of the percentage of centrally nucleated fibers of saline versus α-Klotho infused animals at 14 dpi. Scale: 35 μm. (I, J) Representative images and quantification of MyoD+cells at the site of injury 14 dpi in animals receiving osmotic pump delivery of saline or α-Klotho. Scale: 25 μm. (K) Representative immunofluorescent images showing laminin (red) and DAPI (blue) in animals receiving i.p. administration of saline, α-Klotho 1-3 dpi and α-Klotho 3-5 dpi. Scale: 50 μm. (L) Quantification of percentage of centrally nucleated fibers in aged animals receiving i.p. administration of saline, α-Klotho 1-3 dpi and α-Klotho 3-5 dpi. (M) Quantification of fiber cross-sectional area across the three i.p. injection groups. (N) Fold-change hang impulse score over baseline scores across the 3 i.p. injection groups. (O) Force-frequency curves obtained from in-situ contractile testing analysis of specific force. (n=6-8/group). (*p<0.05, **p<0.01, ***p<0.001, ****p<0.0001).

FIG. 7 shows a graphical abstract. Youthful levels of the circulating hormone α-Klotho are critical for the maintenance of muscle stem cell (MuSC) mitochondrial ultrastructure, which thereby inhibits mtDNA damage and mitochondrial ROS production. This maintenance of healthy mitochondria within MuSCs is required for muscle stem cell activation and contribution to functional skeletal muscle regeneration. However, age-related declines in α-Klotho causes disrupted mitochondrial ultrastructure, increased mtDNA damage, ROS, resulting in an increased senescence, and impaired skeletal muscle regeneration.

FIG. 8 contains a sequence listing of an amino acid sequence (SEQ ID NO:1) of a human klotho precursor polypeptide and a nucleic acid sequence (SEQ ID NO:2) encoding a human klotho precursor polypeptide.

FIG. 9 shows antibody validation. (A) To validate the antibody (MAB1819, R&D Systems) used for histology, muscle sections were co-stained for Klotho and DAPI in wild type and Kl^{-/-} mice. Minimal α-Klotho was detected in the Kl^{-/-} mice. Knockdown of α-Klotho by lentiviral shRNA revealed ˜3-fold decrease in expression in the muscle (B, C) and a decline in circulating Klotho (D). (E, F) MPCs isolated from wild type and Kl^{+/-} micewere co-stained for α-Klotho and DAPI. Immunofluorescence imaging revealed that MPCs from Kl^{+/-} mice expressed ˜50% less α-Klotho. (G, H) In the MPCs, α-Klotho knockdown using a siRNA, revealed ˜3-fold decrease in α-Klotho expression. (I) The ELISA kit (Cloud-Clone Corp, SEH757Mu, Lot#L170622859) was validated by comparing serum levels of α-Klotho from young uninjured (n=8) and Kl^{-/-} mice (n=6). Serum from Kl^{-/-} mice revealed some non-specific binding of the target protein. (J) Intra-assay precision was determined by the coefficient of variation for 7 samples repeated in quintuplicate (*p<0.05, ***p<0.001, ****p<0.0001, student t-test). (A, B, E, G) Scale: 50 μm. Data represented as mean±SEM.

FIG. 10 shows that α-Klotho is also expressed in female muscle with a contusion injury. To confirm that α-Klotho's response to injury is not unique to male mice or cardiotoxin injury, TA muscle sections from a female contusion model were co-stained for α-Klotho and F-actin and imaged using confocal microscopy (Scale: 50 μm).

FIG. 11 shows aging results in a blunted Klotho response following injury in female mice. (A) Klotho expression is increased in young females 3 days post injury (dpi), after which time levels return to the basal state. However, this response is blunted with aging. (B) Demethylation of the Klotho promoter occurs 3 dpi in young female mice, but the response is absent in aged female muscle. (C) There was a decrease in the DNMT3a binding in the young females which is returned to basal binding at 7 and 14 dpi. The reverse trend was observed in aged females. (D) H3K9M2 binding to Klotho promoter declined at 3 dpi and then increased by 14 dpi. However, aged females displayed a decreased H3K9M2 binding to Klotho promoter at 14 dpi. (n=3/group/time point, *p≤0.05 compared to young uninjured muscles; #p≤0.05 indicates a significant difference between young and aged groups at the respective time-point, two-way ANOVA with tukey's post hoc test). Data represented as mean±SEM.

FIG. 12 shows α-Klotho expression in MuSCs and FAPs. (A, B) The intensity of α-Klotho was quantified in a purified population of flow-sorted muscle stem cells (MuSCs), which were subsequently cultured for 6 days. Aged MuSCs display significantly less α-Klotho when compared to young counterparts (Scale: 50 μm ; ****p<0.0001, Student's t-test). (C) Flow sorted MuSCs express Pax7, MyoD and α-Klotho. (D) Confirmation of flow sorted FAPs expressing PDGFRa and α-Klotho. Scale: 50 μm. Data represented as mean±SEM.

FIG. 13 shows decreased α-Klotho expression in MPCs is associated with increased cellular senescence. Aged MPCs display increased senescence as evidenced by an increase in senescence-associated (3-galactosidase expression (A, D; Scale: 100 μm) and increased cytoplasmic expression of HMGB1 (B, E; Scale: 50 μm). When young cells were treated with 25nmol of silencing RNA (siRNA) to α-Klotho, the average percentage of senescent cells was significantly higher when compared to young controls, as determined by SA-f3 gal and cytoplasmic HMGB1. There was no difference in the senescence profiles between old MPCs and young MPCs treated with siRNA to α-Klotho. (C, F) Inhibition of α-Klotho in young MPCs decreased cellular proliferation (Ki67 positivity) (Scale: 50 μm). Klotho supplementation in media while inhibiting Klotho using an siRNA stimulated repair of (G) abasic lesions (n=3) and (H) 8-oxo-dG (n=3). A minimum of 150 cells were analyzed per group for A-F. (****p<0.0001; ***p<0.001; *p≤0.05, one-way ANOVA with tukey's post-hoc test). Data represented as mean±SEM.

FIG. 14 shows that α-Klotho expression does not affect mitochondrial quantity or morphology. (A, B) Confocal and STED microscopy of young, old, young+scramble and young+siRNA MPCs revealed that there is no difference in (C) total mitochondrial volume, (D) volume of each mitochondrion within a cell, (E) the number of mitochondria per cell in any of the groups, or the (F) mitochondrial sphericity (calculated as the ratio of the surface area of the given object to the surface area of a sphere with the same volume as the given object). At least 50 cells per group were analyzed. (p>0.05, one-way ANOVA with tukey's post-hoc test). Data represented as mean±SEM.

FIG. 15 shows that expression of α-Klotho affects the bioenergetics profile of the cells but does not affect the mtDNA copy number. (A, B) Representative bioenergetic profiles for oxygen consumption rate (OCR) and extracellular acidification rate (ECAR) of the MPCs. OCR and ECAR were quantified using a Seahorse XFe96 analyzer. These profiles are representative of eight separate biological repeat experiments performed in 4-6 replicates per run. (C, D) mtDNA copy number in MPCs is not altered with aging or when α-Klotho is knocked-down in young MPCs with an siRNA to α-Klotho. Data represented as mean±SEM.

FIG. 16 shows that SS31 rescues the bioenergetics profile of Kl^{+/-} MPCs to wild-type control levels, but does not significantly alter muscle strength in the absence of injury. (A) Representative bioenergetic profiles for oxygen consumption rate (OCR) of the muscle myoblasts isolated from Kl^{+/-} mice, as determined by Seahorse XFₑ96 analyzer. These profiles are representative of four separate biological repeat experiments performed in 4-6 replicates per run. (B) No significant differences (p>0.05, one-way ANOVA with tukey's post-hoc test) were observed in the hang impulse (weight * number of seconds hanging on the wire) across the three experimental groups at baseline (i.e. prior to injury). Data represented as mean±SEM.

FIG. 17 shows a representative gating strategy used to flow sort MuSCs and FAPs. (A) Sample was gated for live cells as well as a singlet discrimination gate, based on pulse processing parameters. (B) A negative population for CD31 and CD45 was gated on a forward scatter (FSC)/side-scatter (SSC) plot. (C) (CD31+CD45)− population was further gated to determine Sca1⁻+α7 integrin⁺ and Sca1⁺+α7 integrin⁻ populations to yield MuSCs and FAPs, respectively.

FIG. 18 shows size distribution of particles obtained by Zetasizer Nano ZS from: A, brain, B, plasma, and C, CSF. D, Western blotting of isolated fractions from cortex. Homogenate with (CB) and without collagenase (WB) and three fractions were separated by SDS-PAGE and immunoblotted using anti-TSG101, CD81, Kl and ATP5A antibodies.

FIG. 19 shows that human serum-derived exosomes are transporters of Klotho mRNA and protein. (A) Klotho messenger RNA (mRNA) expression levels derived from a public repository of experimentally validated RNA-seq data analyses from the published literature of human blood exosomes of healthy and coronary heart disease patients. (B) Multispectral flow imaging shows that Klotho is expressed in CD63 positive exosomes of murine blood serum. These data suggest that exosomal Klotho cargo may vary in response to (or drive) pathological conditions.

FIG. 20 shows that exosomes express Klotho and exosomal protein gets upregulated with NMES. (A) Confocal imaging shows that Klotho is expressed in CD81 positive exosomes (Scale:20 μm). (B) Protein quantification of exosomes indicates that NMES induces ˜2 times more exosomal proteins in blood serum as compared to serum samples acquired from mice without any rehabilitation or exercise intervention (p=0.08).

FIG. 21 shows that exosomal Klotho gets upregulated with NMES, as analyzed using a Surface Plasmon Resonance (SPR) technique. (A, B) SPR analysis revealed that injected samples were positive for CD31, CD45, Klotho and CD63. Recycling of the injected sample revealed an upregulation of Klotho signal in NMES intervention group only. (C) A subsequent injection of another exosome marker, CD81, confirmed the presence of exosomes carrying the aforementioned markers. (A-C) SPR profile normalized to CD81 indicated that NMES upregulates Klotho carrying exosomes in circulation by ˜2 times compared to control.

FIG. 22 shows Raman Spectroscopy showing a different fingerprint with NMES. (A-C) Raman Spectroscopy analysis on isolated exosomes suggests that there is a difference in the spectra of rehab and no-rehab samples, especially in the Raman shift regions corresponding to phenylalanines and fatty acids. (D) Raman spectra from NMES group can be significantly distinguished from the control samples.

FIG. 23 shows that aged muscle progenitor cells (MPCs) cultured in the presence of young serum display and extracellular vesicle-dependent increase in MyoD expression and bioenergetics. (A) Immunofluorescent imaging of MyoD and DAPI in aged MPCs cultured with serum from aged or young mice. Scale: 25 μm. (B) Quantification of MyoD across the groups (***p<0.001, one-tailed Mann Whitney test, n=73-88 cells/group). (C) Seahorse analysis of oxygen consumption rates (OCR, *p<0.05, one-tailed Student's t test). (D) Immunofluorescent imaging of cardiolipin (NAO) in aged MPCs cultured with young or aged serum. Scale: 50 μm. (E) Quantification of cardiolipin content across groups (*p<0.05, one-tailed Mann Whitney test). (F) Representative nanoparticle tracking curve for nanoparticle concentration of young serum and young serum depleted of extracellular vesicles (EVs) diluted 1:1000. (G) Quantification of MyoD in aged MPCs cultured with EV-depleted serum (****p<0.0001 ##p<0.01 when compared to age-matched controls, one-tailed Student's t test). (H) Representative bioenergetics profile of three independent experiments of aged cells treated with young and aged serum with or without EVs. (I) Seahorse analysis of aged MPCs treated with young and aged serum depleted of EVs (**p<0.01 ##p<0.01 when compared to age-matched controls, one-tailed Student's t test). Seahorse data are represented as the fold change of mean±sem of the three time points prior to oligomycin treatment, performed in 3-8 wells per experiment across three independent experimental setup.

FIG. 24 shows that aging drives a preferential loss of CD63⁺ EV subpopulation. (A) Histogram of concentration of nanoparticles in young and aged serum EVs. (B) ImageStream gating strategy of EVs based on the Root Mean Square gradient histogram. (C) Validation of the best classifier used for computational analysis of EV profile based on parameters such as area under the curve (AUC), classifier accuracy (CA), precision and recall. (D) Confusion matrix for validating machine learning based algorithms. (E) Bubble plot predicting the best EV marker out of CD63, CD81 and CD9 using Gini coefficient (information gain) and χ² (ANOVA) parameters. (F) Quantification of average CD63 expression per EV using ImageStream analysis (****p<0.0001, one-tailed Mann Whitney test, n=4042-5484 EVs/group). (G) Quantification of CD63 expression per EV using in-cell western blot (*p≤0.05, one-tailed Mann Whitney test). (H) Quantification of EV CD63 expression using Surface Plasmon Resonance imaging. Yellow bar indicates end of injection of EVs (*p<0.05, one-tailed Student's t-test, n=4-6/group). (I, J) Quantification of MyoD-positive aged muscle progenitor cells (MPCs) and cardiolipin content of aged MPCs upon exposure to young and aged EVs. (*p<0.05, one-tailed Student's t-test, 97-183 cells/group).

FIG. 25 shows that aging results in a distinct biochemical fingerprint of circulating EVs. (A) Average Raman spectra with standard deviation (grey band) of young and aged serum EVs. (B) Subtraction spectrum of the differences between the average spectra acquired for young and aged serum EVs. (C) Quantification of protein content per nanoparticle isolated using BCA assay. (D) Principal Component Analysis (PCA) with confidence interval of 95% and (E) Linear Discriminant Analysis of data acquired from aged and young serum EVs (n=5/group; ***p<0.001, Mann Whitney test).

FIG. 26 shows that Klotho mRNAs are preferentially contained within CD63⁺ EVs in an age-dependent manner. (A) Immunofluorescent imaging of Klotho and DAPI in aged muscle progenitor cells (MPCs) cultured in the presence or absence of young EVs in culturing media. Scale: 50 μm. (B) Quantification of Klotho in aged MPCs treated with and without EVs (***p<0.001, one-tailed Mann Whitney test, n=66-119 cells/group). (C) ELISA based quantification of Klotho in conditioned media of aged MPCs treated with and without young EVs (*p<0.05, one-tailed Student's t test, n=7-8/group). (D) Quantification of Klotho in aged MPCs receiving EVs isolated from Kl^{+/+} and Kl^{+/-} serum (*p<0.05, one-tailed Welch's t test, n=126-144/group). (E) Surface Plasmon Resonance imaging (SPRi) analysis of Klotho protein in young and aged serum EVs (p>0,05, one-tailed Student's t test, n=4-7/group). (F) TSNE maps of Klotho mRNA distribution within the CD63⁺ EVs and ImageStream analysis on Klotho mRNA/EV (**p<0.01, one-tailed Student's t test, n=22605-28338 EVs/group). (G) Quantification of Klotho mRNA in young and aged EVs based on digital PCR. Data represent the total Klotho mRNA abundance after pooling of four different samples. (H) Histogram representing the preferential storage of Klotho mRNA in CD63⁺ EVs (***p<0.001, one-tailed Student's t test). (I) Quantification of Klotho protein in Kl^{-/-} MPCs receiving young EVs and young EVs treated with siRNA to Klotho (***p<0.001, one-tailed Mann Whitney test, n=68-104 cells/group). Datasets of Kl^{-/-} MPCs treated with young EVs and EVs treated with non-targeting siRNA were pooled since there was no statistical difference between the two groups.

FIG. 27 shows the beneficial effect of young serum injections on tibialis anterior muscle contractile force production 11 days post injury which is abrogated when serum is depleted of EVs. A total of eight serum injections were administered to aged animals via tail vein every three days. Please note that the dotted lines are average of our historical data of specific force of young and aged animals, 14 days post injury. (**p<0.01, one-tailed Student's t-test, n=6/group).

FIG. 28 shows that Klotho mRNA within EVs contribute to the functional regeneration of aged animals. (A) Schematic of the in vivo administration of EVs to injured aged mice. (B, C) Histological analysis of fiber cross-sectional area of injured TAs of aged mice receiving young EVs. (*p<0.05, Mann Whitney test, n=5-8/group). (D) Quantification of specific tetanic force at 100 Hz for control aged animals and aged animals receiving intramuscular injection of young EVs when compared to saline-injected controls (*p<0.05, Mann Whitney test, n=5-11/group). (E) Specific peak tetanic force of aged animals receiving EVs isolated from Klotho^{+/+} or Klotho^{+/-} serum (p=0.06, one-tailed Student's t test, n=6/group).

FIG. 29 shows an in-cell western blot image supporting the analysis in FIG. 2G.

FIG. 30 demonstrates the impact of EV age on target cell Klotho protein expression. (A) Quantification of Klotho protein in aged muscle progenitor cells following culture in the presence of young or aged EVs over 24 hours.

FIG. 31 shows the quantification of relative abundance Klotho mRNA EVs using digital PCR. (A) Quantification of Klotho mRNA in EVs isolated from young Klotho^{+/-} mice as compared to young Klotho mice. (B) Quantification of Klotho mRNA in young EVs treated with siRNA to Klotho and compared to young serum EVs. Data is representative of 4 independent samples pooled together for digital PCR analysis.

FIG. 32 shows the overall body endurance of animals used in the study. Overall body endurance as determined by the hanging grid test is variable one-day post injury (1 dpi) as revealed by the hang-grid impulse scores at 1 dpi normalized to baseline scores. Only animals having a score within 25-75% percentile of the median (range: 0.40-0.74) were included in the study.

FIG. 33 shows an example of engineering EVs with synthetic Klotho mRNA. (A) Representative images of aged MPCs incubated in EVs isolated from Kl+/− mice(Kl+/− EVs) or Kl+/− EVs that have been loaded with synthetic Klotho mRNA (Kl+/− EVs +KL). Scale: 50 μm. (B) Quantification of cytosolic Klotho protein expression after incubating aged MPCs with engineered Kl^{+/-} EVs or Kl^{+/-} EVs loaded with Klotho mRNA (KL) (**p<0.01, one-tailed Student's t-test). (C) Quantification of specific tetanic force at 100 HZ of tibialis anterior muscles from aged animals that received saline intramuscular injections or aged animals that received EVs isolated from Kl^{+/-} mice and subsequently loaded with synthetic KL mRNA (p=0.07, one-tailed Student's t-test). Contractile force measurements were taken two weeks after injury.

FIG. 34 is an exemplary nucleic acid sequence encoding a human Klotho polypeptide (SEQ ID NO:3). The start site (ATG) is bolded. A region containing methylation sites within the 5′ sequence is underlined (SEQ ID NO:4).

### DETAILED DESCRIPTION

This document provides methods and materials for treating aging (e.g., for treating aged mammals). For example, a mammal having, or at risk for developing, an age-related impairment (e.g., sarcopenia and/or age-related cognitive decline) can be treated by increasing the level of one or more myokine polypeptides (e.g., one or more Klotho polypeptides) in one or more cells within the mammal. In some cases, one or more myokine polypeptides (e.g., one or more Klotho polypeptides) can be administered to a mammal having, or at risk for developing, an age-related impairment (e.g., sarcopenia and/or age-related cognitive decline) to treat the mammal. In some cases, nucleic acid encoding one or more myokine polypeptides (e.g., nucleic acid encoding one or more Klotho polypeptides) can be administered to a mammal having, or at risk for developing, an age-related impairment (e.g., sarcopenia and/or age-related cognitive decline) to treat the mammal. In some cases, an exosome containing (a) one or more myokine polypeptides (e.g., one or more Klotho polypeptides) and/or (b) nucleic acid (e.g., mRNA) encoding one or more myokine polypeptides (e.g., one or more Klotho polypeptides) can be administered to a mammal having, or at risk for developing, an age-related impairment (e.g., sarcopenia and/or age-related cognitive decline) to treat the mammal. In some cases, a gene editing system designed to reduce or eliminate methylation of a promoter that directs expression of a myokine polypeptide (e.g., an α-Klotho polypeptide) can be administered to a mammal having, or at risk for developing, an age-related impairment (e.g., sarcopenia and/or age-related cognitive decline) to treat the mammal.

This document also provides methods and materials for increasing the ability of stem cells (e.g., MPCs) to regenerate more differentiated cells (e.g., muscle cells) within a mammal (e.g., a human) having an impairment or injury (e.g., a muscle impairment). For example, one or more myokine polypeptides (e.g., one or more Klotho polypeptides such as an α-Klotho polypeptide), nucleic acid encoding one or more myokine polypeptides (e.g., nucleic acid encoding one or more Klotho polypeptides such as an α-Klotho polypeptide), exosomes containing one or more myokine polypeptides and/or nucleic acid encoding one or more myokine polypeptides, and/or a gene editing system designed to reduce or eliminate methylation of a promoter that directs expression of a myokine polypeptide (e.g., an α-Klotho polypeptide) can be used to increase the ability of stem cells (e.g., MPCs) to regenerate more differentiated cells (e.g., muscle cells) within a mammal (e.g., a human) having an impairment or injury (e.g., a muscle impairment) to treat that an impairment or injury. Examples of muscle impairments that can be treated as described herein include, without limitation, sarcopenia, traumatic muscle injury, myopathies, and post-surgical muscle injury.

In general, the ability of stem cells (e.g., MPCs) to regenerate more differentiated cells (e.g., muscle cells) within a mammal (e.g., a mammal having a muscle impairment) can be increased by increasing the level of one or more myokine polypeptides (e.g., a Klotho polypeptide such as an α-Klotho polypeptide) within a muscle progenitor cell. The level of one or more myokine polypeptides (e.g., a Klotho polypeptide such as an α-Klotho polypeptide) within a stem cell (e.g., an MPC) can be increased using any of the methods or materials described herein. For example, the methods or materials described herein for increasing the level of one or more myokine polypeptides (e.g., a Klotho polypeptide such as an α-Klotho polypeptide) to treat an age-related impairment (e.g., sarcopenia and/or age-related cognitive decline) can be applied to stem cells (e.g., MPCs) to increase the ability of stem cells (e.g., MPCs) to regenerate more differentiated cells (e.g., muscle cells) within a mammal. The level of one or more myokine polypeptides (e.g., a Klotho polypeptide such as an α-Klotho polypeptide) within a stem cell (e.g., an MPC) can be increased by administering one or more myokine polypeptides (e.g., one or more Klotho polypeptides) to a mammal having stem cells, by administering nucleic acid (e.g., mRNA) encoding one or more myokine polypeptides (e.g., one or more Klotho polypeptides) to a mammal having stem cells, and/or by administering an exosome containing (a) one or more myokine polypeptides (e.g., one or more Klotho polypeptides) and/or (b) nucleic acid (e.g., mRNA) encoding one or more myokine polypeptides (e.g., one or more Klotho polypeptides) to a mammal having stem cells. The administration of exosomes containing (a) one or more myokine polypeptides (e.g., one or more Klotho polypeptides) and/or (b) nucleic acid (e.g., mRNA) encoding one or more myokine polypeptides (e.g., one or more Klotho polypeptides) can result in the exosomes crossing the blood brain barrier to deliver the contents to cells (e.g., neurons, microglia, and astrocytes) within the brain of a mammal (e.g., a human).

Examples of stem cells that can be treated as described herein to increase regenerative capabilities include, without limitation, epithelial stem cells, MPCs, neuronal stem cells, and hematopoietic stem cells. In some cases, increasing the ability of epithelial stem cells to regenerate epithelial cells can be used to improve wound healing. In some cases, the methods and materials described herein can be used to increase the ability of aged stem cells (e.g., aged MPCs) to regenerate more differentiated cells (e.g., muscle cells) within a mammal (e.g., a human) having an impairment or injury (e.g., a muscle impairment). For example, the methods and materials described herein can be used to increase the ability of aged stem cells (e.g., aged MPCs) to regenerate more differentiated cells (e.g., muscle cells) within a human that is over the age of 20, 30, 40, 50, 60, or 70 years and has an impairment or injury (e.g., a muscle impairment).

The term “increased level” as used herein with respect to a level of a myokine polypeptide (e.g., a Klotho polypeptide) refers to any level that is greater than the median level of that myokine polypeptide as typically observed in a mammal that does not have an age-related impairment. Control samples can include, without limitation, samples from young mammals. It will be appreciated that levels from comparable samples or tissues are used when determining whether or not a particular level is an increased level.

Any appropriate mammal having, or at risk for developing, an age-related impairment can be treated as described herein. Examples of mammals having, or at risk for developing, an age-related impairment that can be treated as described herein (e.g., by increasing the level of one or more myokine polypeptides such as Klotho polypeptides in one or more cells within the mammal) include, without limitation, humans, non-human primates (e.g., monkeys), dogs, cats, horses, cows, pigs, sheep, mice, and rats. For example, a human having, or at risk for developing, an age-related impairment can be treated by increasing the level of one or more myokine polypeptides such as Klotho polypeptides in one or more cells within that human.

When treating a mammal (e.g., a human) having, or at risk for developing, an age-related impairment as described herein (e.g., by increasing the level of one or more myokine polypeptides such as Klotho polypeptides in one or more cells within the mammal), the age-related impairment can be any type of age-related impairment. In some cases, an age-related impairment can be associated with reduced or eliminated levels of one or more myokine polypeptides. For example, an age-related impairment can be associated with methylation of a promoter that directs expression of one or more myokine polypeptides such that the methylated promoter results in reduced or eliminated levels of one or more myokine polypeptides. In some cases, an age-related impairment can be a degenerative disease or condition. Examples of age-related impairments include, without limitation, declines in cellular regeneration, cognitive declines, atrophy, wound healing, arteriosclerosis, osteoporosis, muscle impairments associated with aging (e.g., sarcopenia), and impaired regenerative responses. An age-related impairment can affect any part of a mammal (e.g., any part of a mammal's body). Examples of parts of a mammal that can be affected by an age-related impairment include, without limitation, muscles (e.g., skeletal muscles, smooth muscles, and cardiac muscles), blood vessels (e.g., arteries), nerves, bones, or skin. In some cases, an age-related impairment can be an age-related decline in muscle regeneration (e.g., impaired muscle regeneration). In some cases, an age-related impairment can be an age-related cognitive decline (e.g., impaired cognitive function).

In some cases, methods described herein can include identifying a mammal (e.g., a human) as having, or as being at risk for developing, an age-related impairment. Any appropriate method can be used to identify a mammal as having an age-related impairment. For example, a reduced level of one or more myokine polypeptides in a sample obtained from a mammal can be used to identify a mammal having an age-related impairment. For example, the presence of methylation on a promoter that directs expression of a myokine polypeptide (e.g., a methylated myokine promoter such as a methylated Klotho promoter) in a sample obtained from a mammal can be used to identify a mammal having an age-related impairment. A sample can be any appropriate sample. In some cases, a sample can be a fluid sample (e.g., a blood sample). In some cases, a sample can be tissue sample (e.g., a biopsy). Examples of samples that can be obtained from a mammal and assessed for a reduced level of one or more myokine polypeptides and/or the presence of methylation on myokine promoter include, without limitation, blood samples (e.g., whole blood, serum, and plasma), muscle tissue samples, and urine samples.

Once identified as having, or as being at risk for developing, an age-related impairment, a mammal (e.g., a human) can be administered, or instructed to self-administer, one or more myokine polypeptides (e.g., one or more Klotho polypeptides). For example, one or more myokine polypeptides can be administered to a mammal in need thereof (e.g., a mammal having, or at risk for developing, an age-related impairment). For example, a human having, or at risk for developing, an age-related impairment can be treated by administering one or more myokine polypeptides, nucleic acid encoding one or more myokine polypeptides, and/or a gene editing system designed to reduce or eliminate methylation of a promoter that directs expression of one or more myokine polypeptides to increase the level of one or more myokine polypeptides such as Klotho polypeptides in one or more cells within that human.

In some cases, one or more myokine polypeptides (e.g., one or more Klotho polypeptides) can be administered to a mammal having, or at risk for developing, an age-related impairment to treat the mammal. For example, a mammal having, or at risk for developing, an age-related impairment can be administered or can self-administer a composition containing one or more myokine polypeptides. In some cases, a composition containing one or more myokine polypeptides (e.g., a composition containing a Klotho polypeptide) can be administered to a mammal having an age-related impairment to increase the level of one or more myokine polypeptides (e.g., a Klotho polypeptide) within that human.

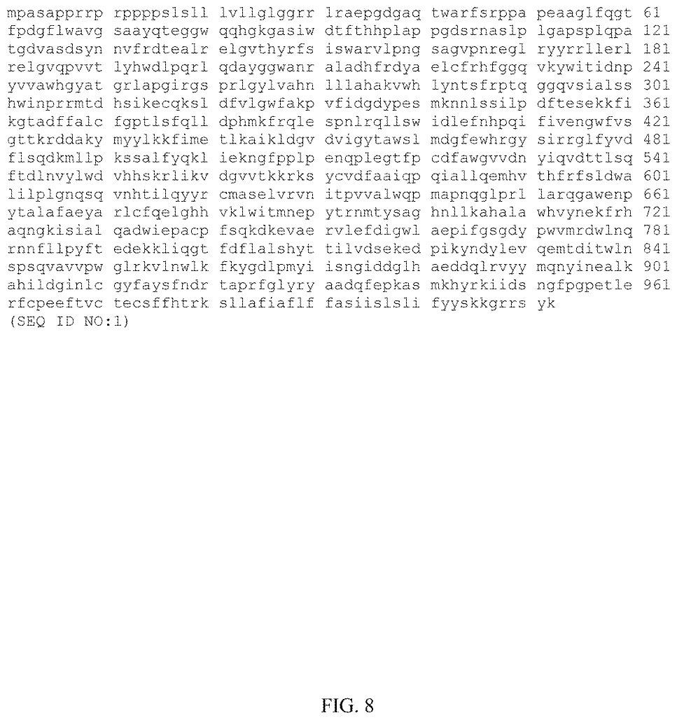

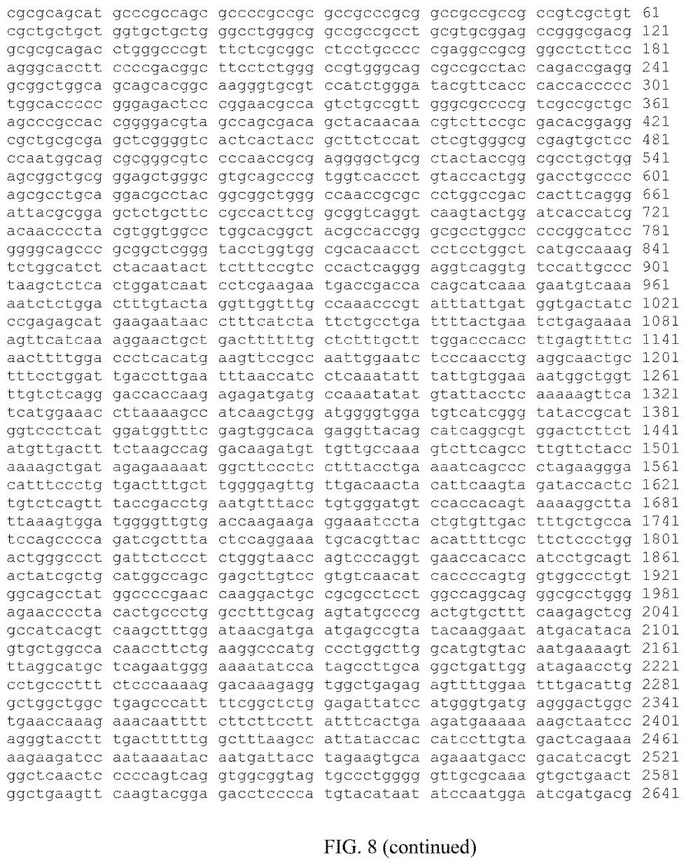

A myokine polypeptide can be any appropriate myokine polypeptide. In some cases, a myokine polypeptide can be an anti-aging myokine. In some cases, a myokine polypeptide can be an exercise-induced myokine polypeptide (e.g., a myokine polypeptide whose cellular expression of the myokine polypeptide is driven by physical exertion and/or skeletal muscle contraction). In some cases, a myokine polypeptide can be a circulating myokine polypeptide (e.g., a myokine polypeptide present in the bloodstream of a mammal). In some cases, a myokine polypeptide can be from about 5 kDa to about 140 kDa (e.g., from about 5 kDa to about 135 kDa, from about 15 kDa to about 140 kDa, from about 50 kDa to about 140 kDa, or from about 120 kDa to about 135 kDa). In some cases, a myokine polypeptide can have autocrine, paracrine and/or endocrine effects. Examples of myokine polypeptides that can be used as described herein (e.g., to treat a mammal having, or at risk for developing, an age-related impairment) include, without limitation, interleukins (ILs; e.g., IL-6), Klotho (e.g., α-Klotho, β-Klotho, and γ-Klotho), GDF-11, and brain-derived neurotrophic factor (BDNF). For example, a mammal having, or at risk for developing, an age-related impairment can be administered or can self-administer one or more Klotho polypeptides (e.g., one or more α-Klotho polypeptides). An example of a Klotho polypeptide that can be used as described herein includes, without limitation, a human Klotho polypeptide having the amino acid sequence set forth in National Center for Biotechnology Information (NCBI) GenBank® Accession No. NP_004786.2. A representative human Klotho polypeptide sequence is set forth in FIG. 8 as SEQ ID NO:1.

In some cases, once identified as having, or as being at risk for developing, an age-related impairment, a mammal (e.g., a human) can be administered, or instructed to self-administer, nucleic acid encoding one or more myokine polypeptides (e.g., one or more Klotho polypeptides). For example, nucleic acid encoding one or more myokine polypeptides (e.g., one or more Klotho polypeptides) can be administered to a mammal having, or at risk for developing, an age-related impairment to treat the mammal. In some cases, nucleic acid encoding one or more myokine polypeptides can be administered to a mammal having, or at risk for developing, an age-related impairment to increase the level of one or more myokine polypeptides such as Klotho polypeptides in one or more cells within that human.

A nucleic acid encoding a myokine polypeptide (e.g., a Klotho polypeptide) can be any appropriate nucleic acid. A nucleic acid encoding a myokine polypeptide can encode any myokine polypeptide described herein. In some cases, a nucleic acid encoding a myokine polypeptide can encode a Klotho polypeptide (e.g., an α-Klotho polypeptide). An example of a nucleic acid encoding a Klotho polypeptide includes, without limitation, nucleic acid encoding a human Klotho sequence as set forth in GenBank® Accession No. NM_004795.3. A representative nucleic acid sequence encoding a human Klotho polypeptide is set forth in FIG. 8 as SEQ ID NO:2.

In some cases, a nucleic acid encoding a myokine polypeptide (e.g., a Klotho polypeptide) can be in a nucleic acid vector (e.g., an expression vector). In some cases, a vector can be a plasmid. In some cases, a vector can be viral vector (e.g., a lentiviral vector). Examples of viral vectors that can be used to deliver nucleic acid encoding one or more myokine polypeptides (e.g., a Klotho polypeptide) to a mammal to treat an age-related impairment as described herein include, without limitation, adenoviral vectors, adeno-associated viral vectors (e.g., AAV8 viral vectors and chimeric AAV2/AAV8 viral vectors), lentiviral vectors, herpes viral vectors, retroviral vectors, and vaccinia viral vectors.

An expression vector (e.g., viral vector) can include one or more elements necessary for expressing a polypeptide (e.g., a myokine polypeptide) from a nucleic acid sequence within the vector (e.g., a ribosomal binding site and start codon, a termination codon, and a transcription termination sequence). In cases where a nucleic acid encoding a myokine polypeptide is a vector, the vector also can include one or more regulatory elements (e.g., enhancers and promotes) that can enhance expression of a polypeptide (e.g., a myokine polypeptide) from a nucleic acid sequence within the vector. A promoter can be a constitutive promoter or an inducible promoter. A promoter can be a ubiquitous promoter or a tissue/cell-specific promoter (e.g., a muscle-specific promoter). An example of a promoter that can increase expression of a polypeptide (e.g., a myokine polypeptide) from a nucleic acid sequence within a vector includes, without limitation, a Pitx3 muscle-specific promoter.

In cases where a nucleic acid encoding a myokine polypeptide is a vector, the vector also can include an origin of replication, a selectable marker, and/or a nucleic acid encoding a detectable label.

In some cases, once identified as having, or as being at risk for developing, an age-related impairment, a mammal (e.g., a human) can be administered, or instructed to self-administer, exosomes containing (a) one or more myokine polypeptides (e.g., one or more Klotho polypeptides) and/or (b) nucleic acid encoding one or more myokine polypeptides (e.g., one or more Klotho polypeptides). For example, exosomes containing (a) one or more myokine polypeptides (e.g., one or more Klotho polypeptides) and/or (b) nucleic acid encoding one or more myokine polypeptides (e.g., one or more Klotho polypeptides) can be administered to a mammal in need thereof (e.g., a mammal having, or at risk for developing, an age-related impairment). For example, a human having, or at risk for developing, an age-related impairment can be treated by administering exosomes containing (a) one or more myokine polypeptides (e.g., one or more Klotho polypeptides) and/or (b) nucleic acid encoding one or more myokine polypeptides (e.g., one or more Klotho polypeptides). In some cases, an exosome can contain mRNA encoding one or more myokine polypeptides (e.g., one or more Klotho polypeptides).

Any appropriate method can be used to obtain exosomes containing (a) one or more myokine polypeptides (e.g., one or more Klotho polypeptides) and/or (b) nucleic acid encoding one or more myokine polypeptides (e.g., one or more Klotho polypeptides). For example, cells (e.g., muscle cells) in cultured in a manner to accumulate exosomes in culture. In some cases, the cultured cells can be genetically manipulated to express one or more myokine polypeptides (e.g., one or more Klotho polypeptides) from exogenously added nucleic acid. Once produced, the exosomes can be isolated from the cell culture supernatants. Any appropriate method can be used to obtain or enrich a sample for exosomes. For example, cell sorting techniques can be used to obtain particular exosomes.

In some cases, exosomes isolated from cells can be treated in a manner that loads particular contents into the exosome. For example, engineered skeletal muscle or muscle stem cell exosomes can be loaded with exogenous myokine polypeptides (e.g., an α-Klotho polypeptide) or nucleic acid encoding a myokine polypeptide (e.g., nucleic acid encoding an α-Klotho polypeptide). In some cases, exosomes can be designed to have one or more rabies virus glycoprotein (RVG) peptides attached to their surface to deliver the myokine cargo to neurons, microglia, and oligodendrocytes after administration (e.g., an intravenous injection). Other exosome surface modifications or attachments of molecules on exosomal surfaces can be used to fine tune the stability of the exosomes in vivo, the pharmacokinetics of the exosomes, and/or the biodistribution of the exosomes.

In some cases, synthetically generated vesicles can be used in place of exosomes. For example, a synthetically generated vesicle having dimensions similar to those of exosomes can be made to contain (a) one or more myokine polypeptides (e.g., one or more Klotho polypeptides) and/or (b) nucleic acid encoding one or more myokine polypeptides (e.g., one or more Klotho polypeptides) and used as described herein. Any appropriate method can be used to make synthetically generated vesicles containing (a) one or more myokine polypeptides (e.g., one or more Klotho polypeptides) and/or (b) nucleic acid encoding one or more myokine polypeptides (e.g., one or more Klotho polypeptides). For example, high payloads of (a) one or more myokine polypeptides (e.g., one or more Klotho polypeptides) and/or (b) nucleic acid encoding one or more myokine polypeptides (e.g., one or more

Klotho polypeptides) can be stably accommodated by several synthetic liposomal formulations or exosomes-mimetics of semi-synthetic origin (e.g., using lipids extracted from specific tissues, cells, or extracellular vesicles) that overcome cellular/tissue uptake obstacles and promote better biodistribution. In some cases, synthetic liposomal formulations or exosomes-mimetics can provide efficient systemic delivery within a mammal's brain or other target distal organs in a manner that minimizes any collateral systemic side effects.

In some cases, an exosome (and/or synthetically generated vesicle) containing (a) one or more myokine polypeptides (e.g., one or more Klotho polypeptides) and/or (b) nucleic acid encoding one or more myokine polypeptides (e.g., one or more Klotho polypeptides) can be administered systemically to deliver contents to cells within a mammal's brain or other distal organs.

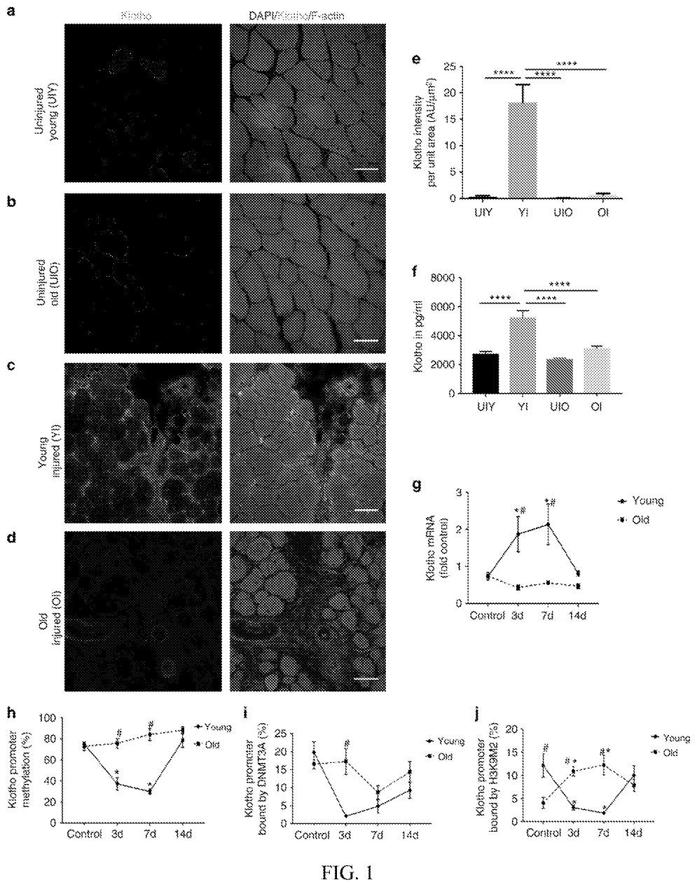

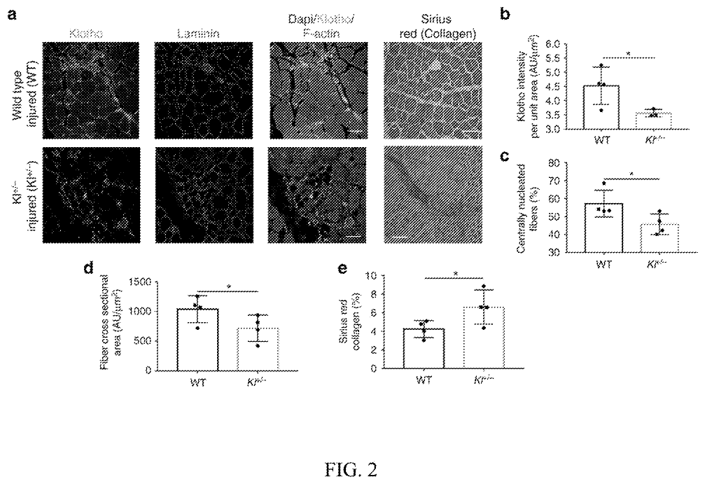

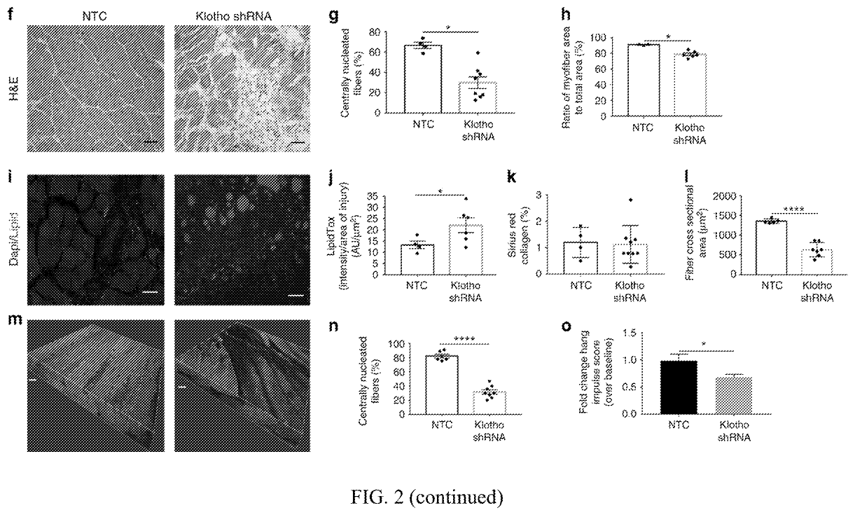

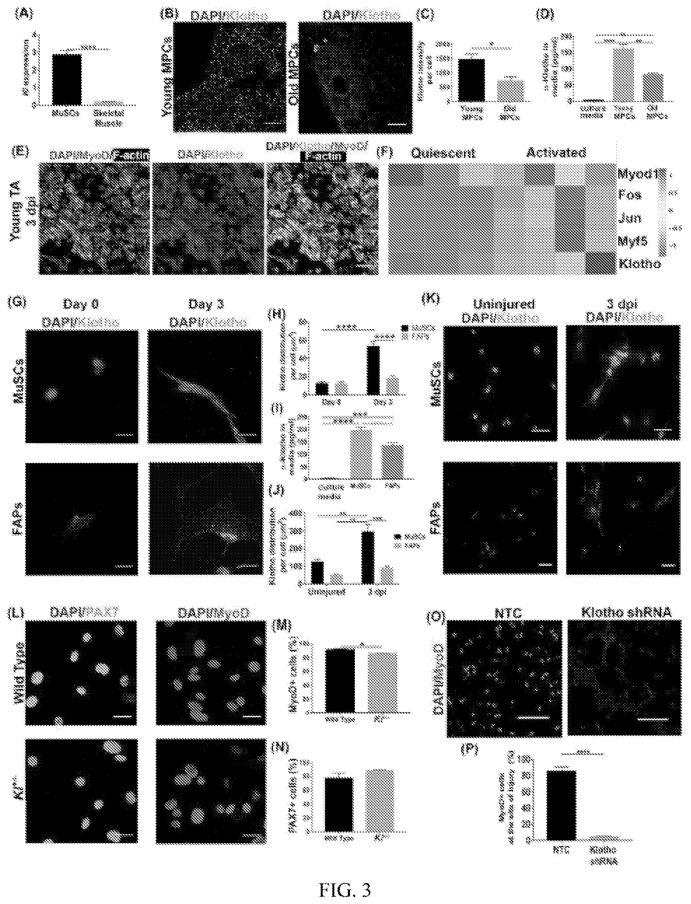

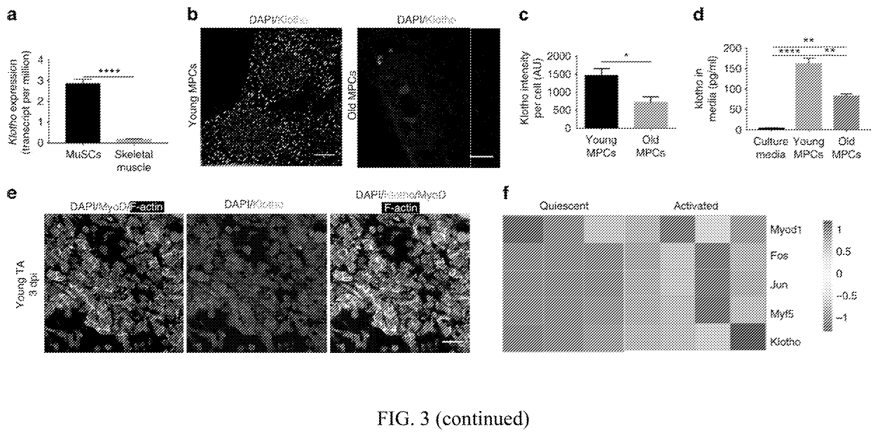

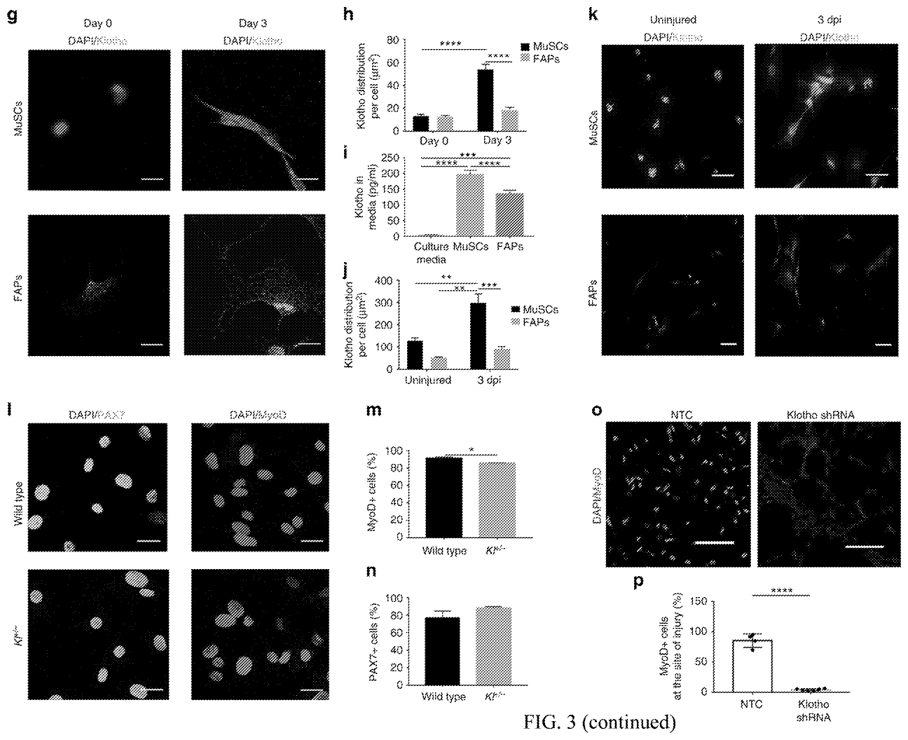

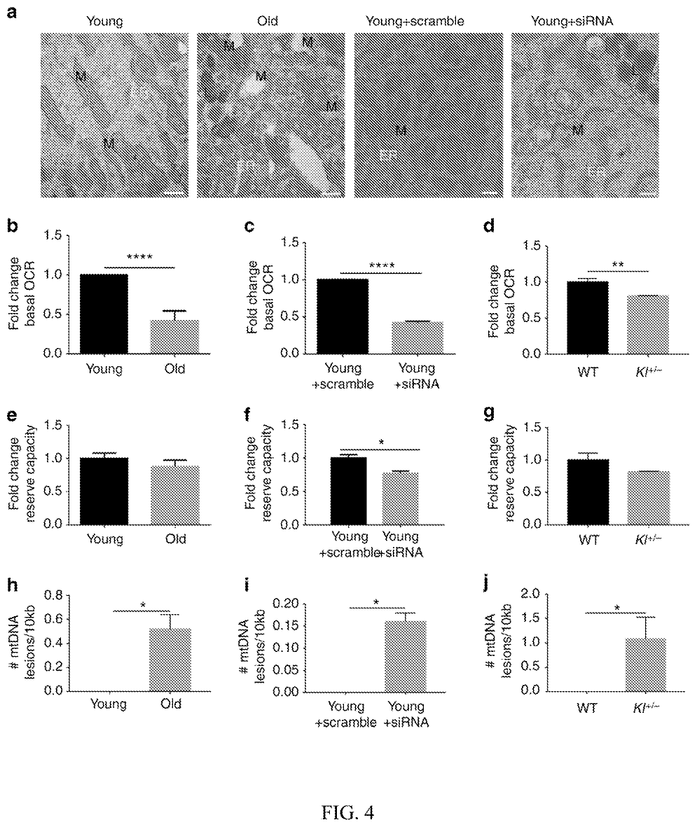

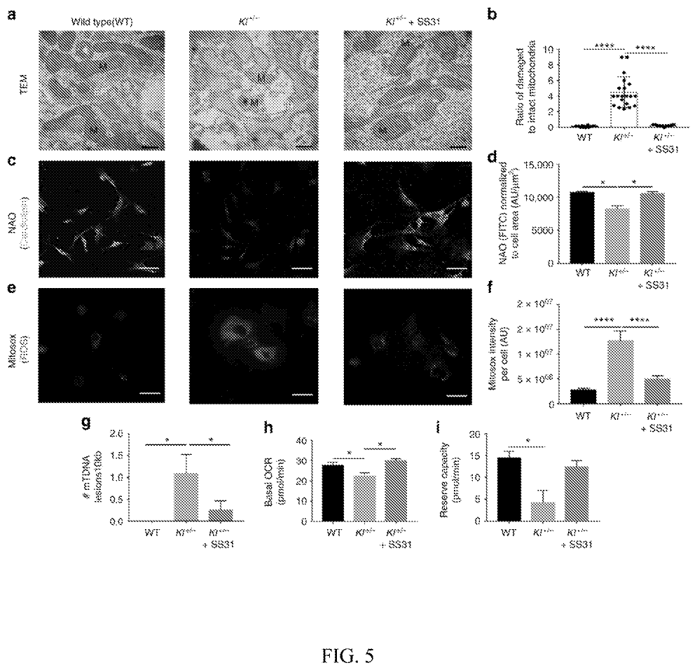

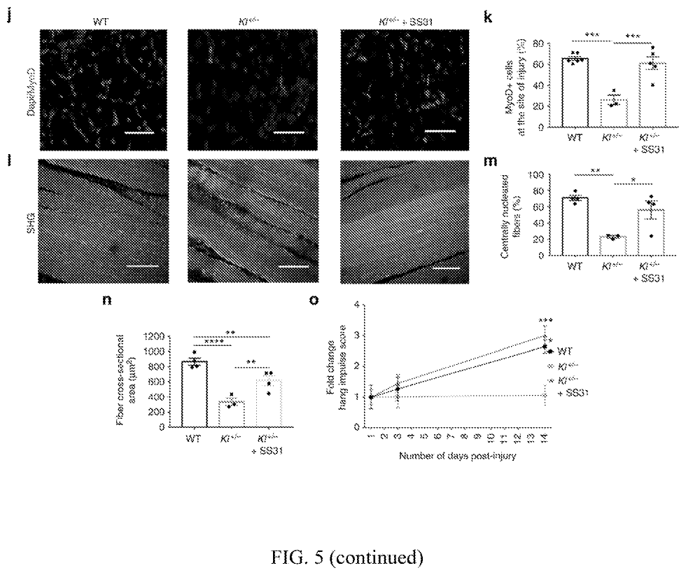

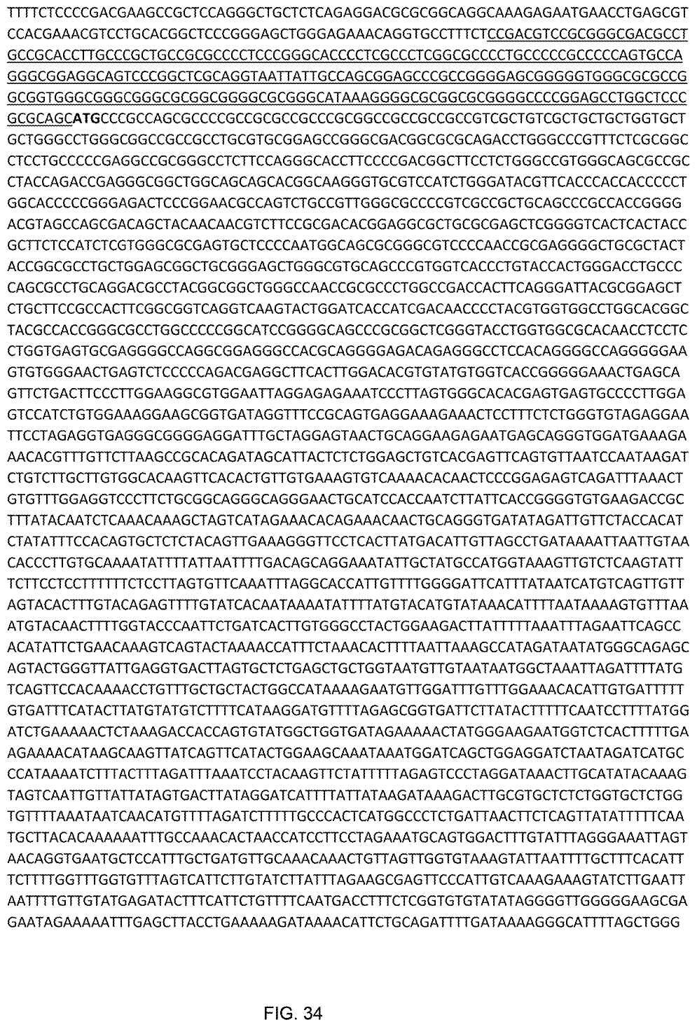

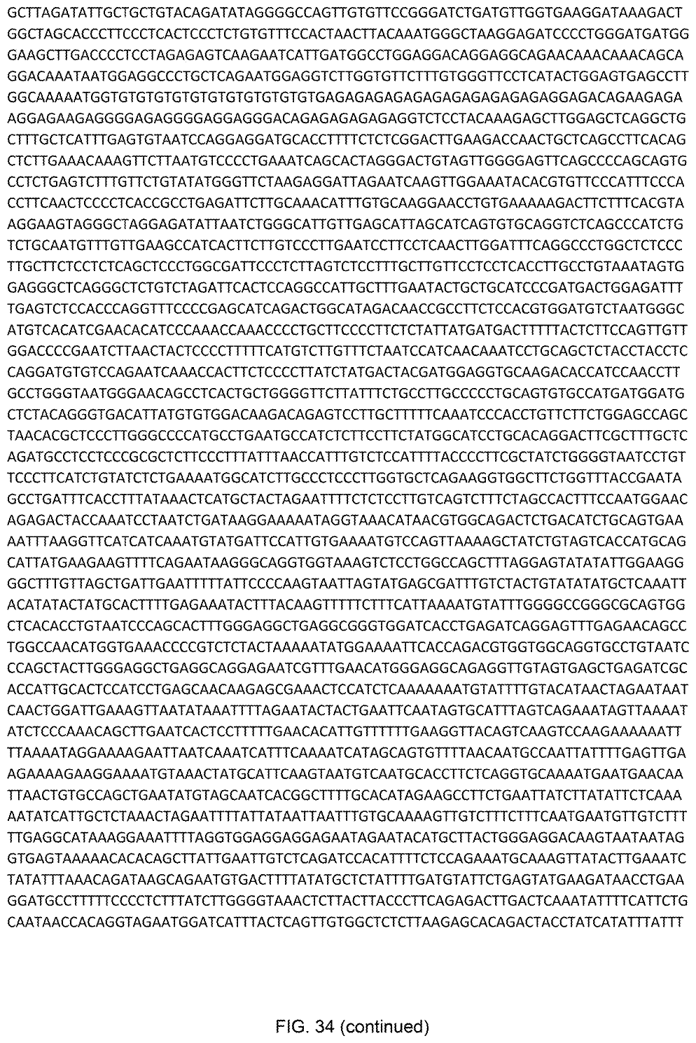

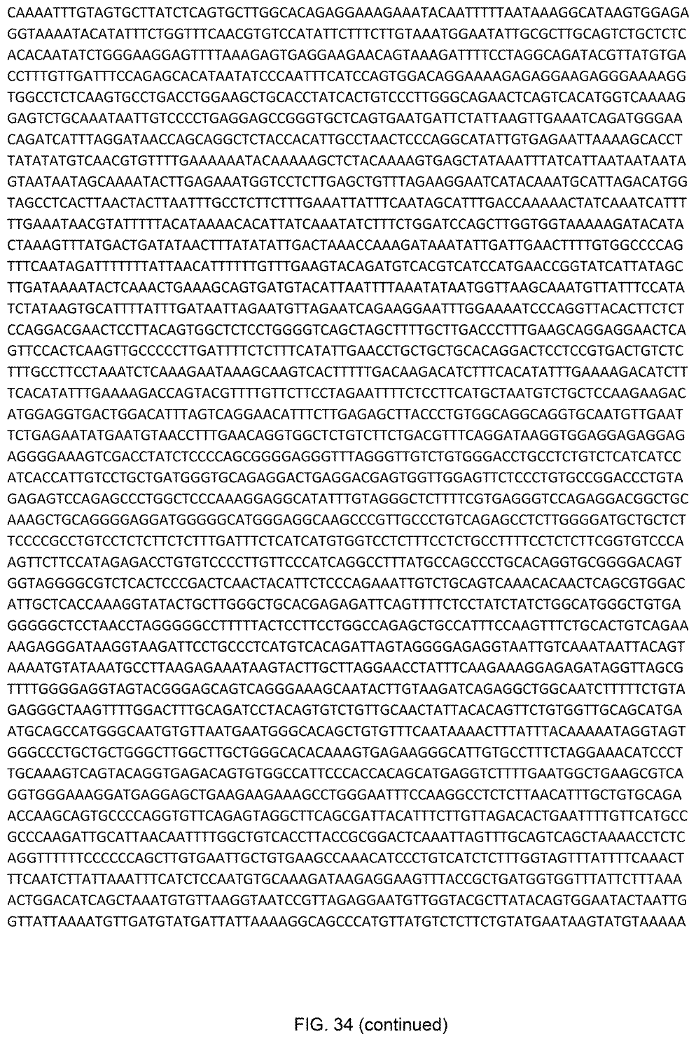

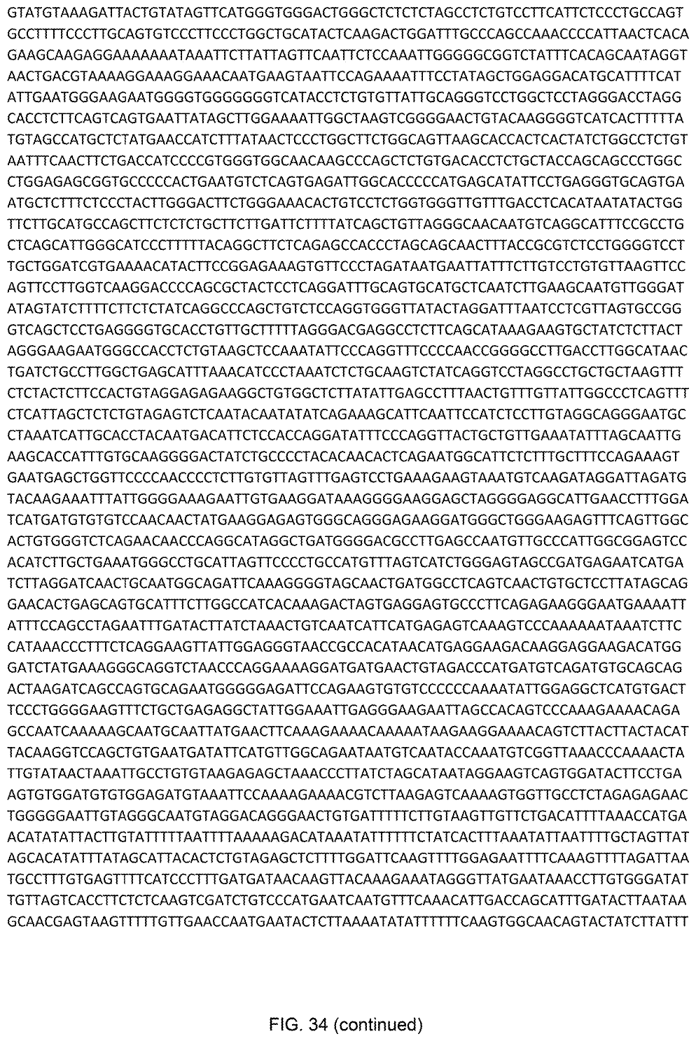

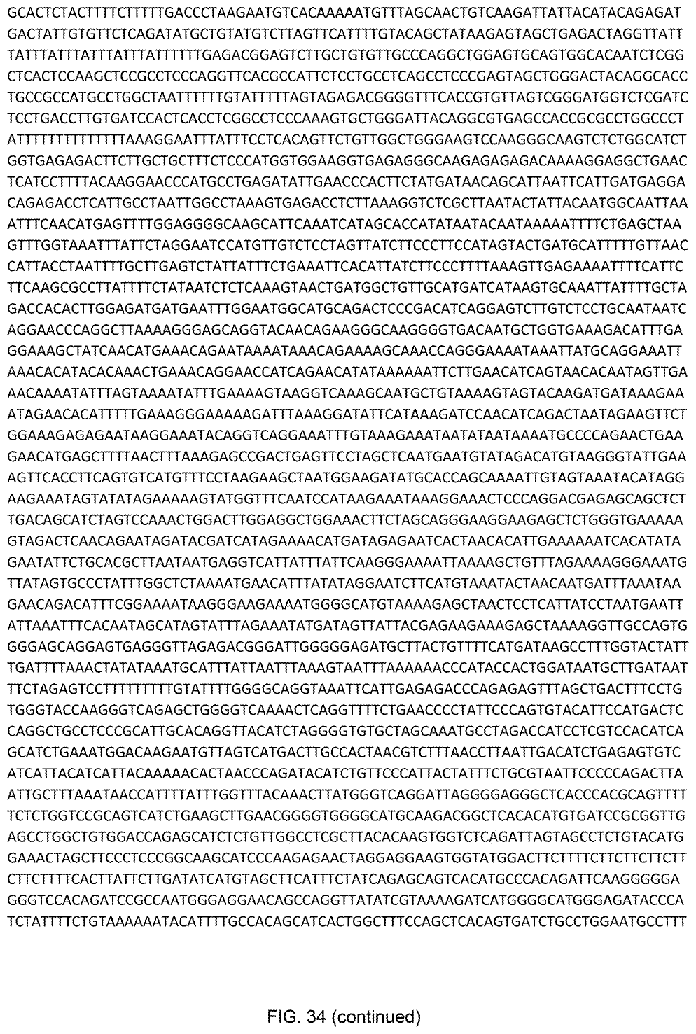

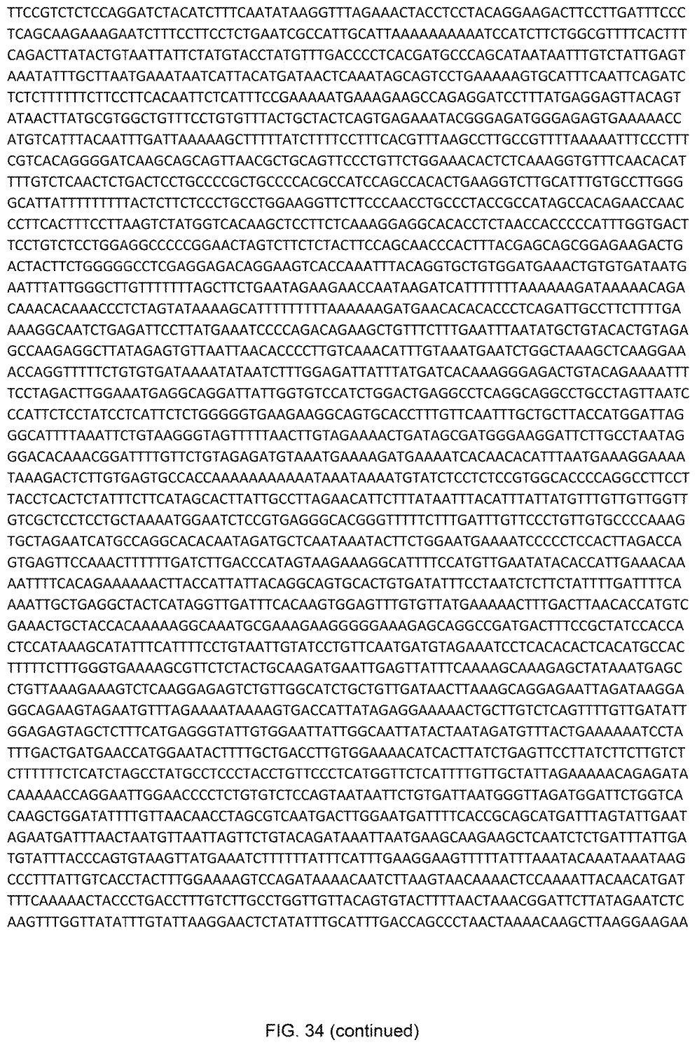

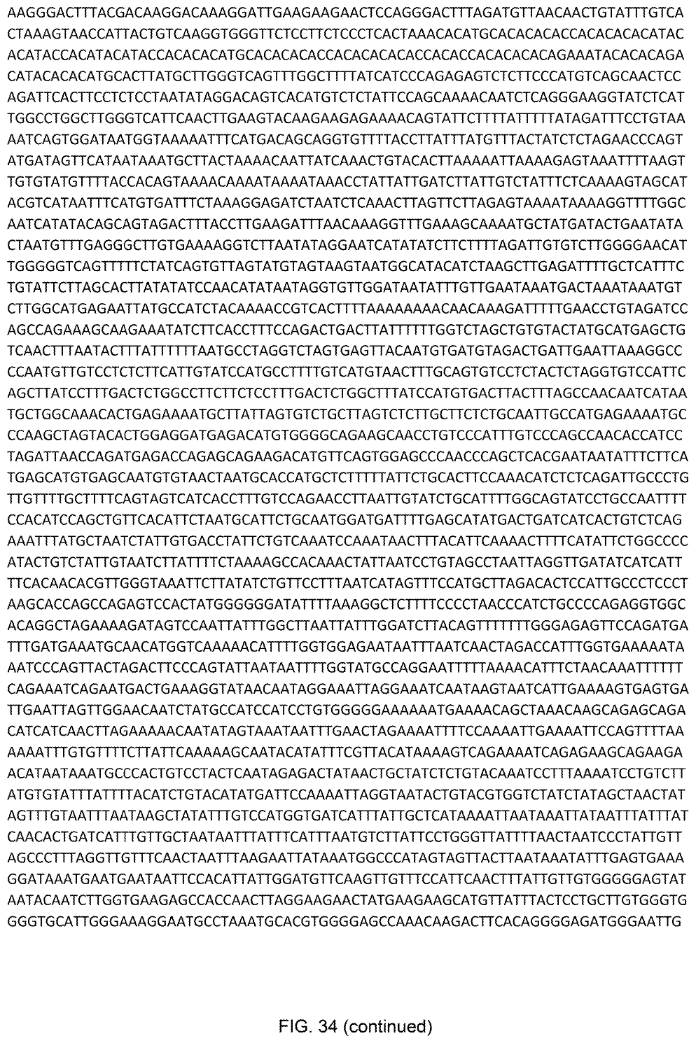

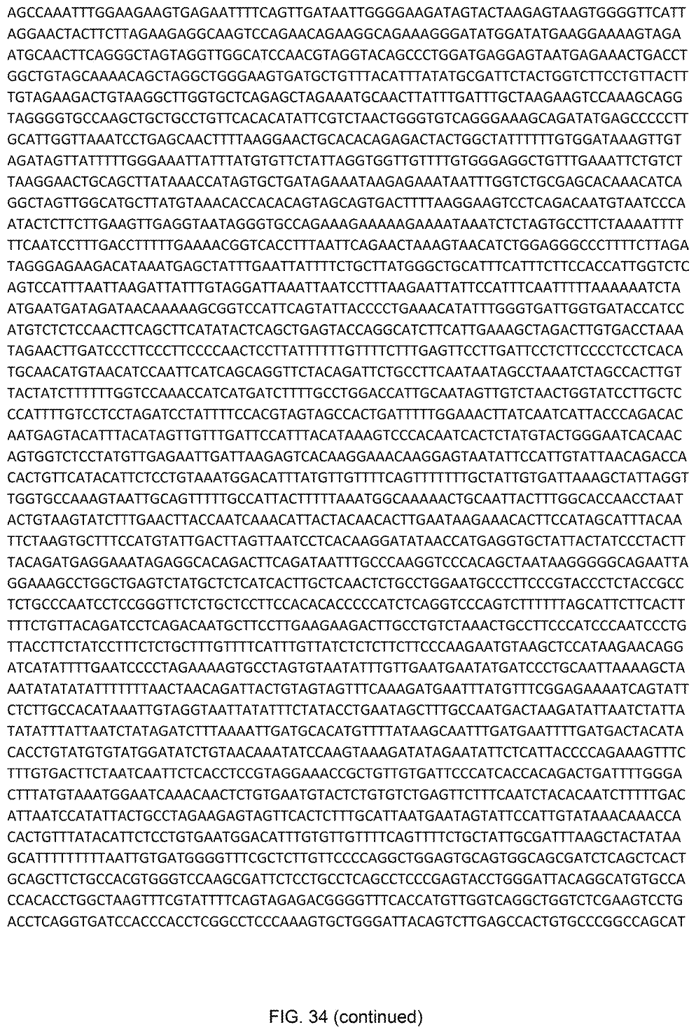

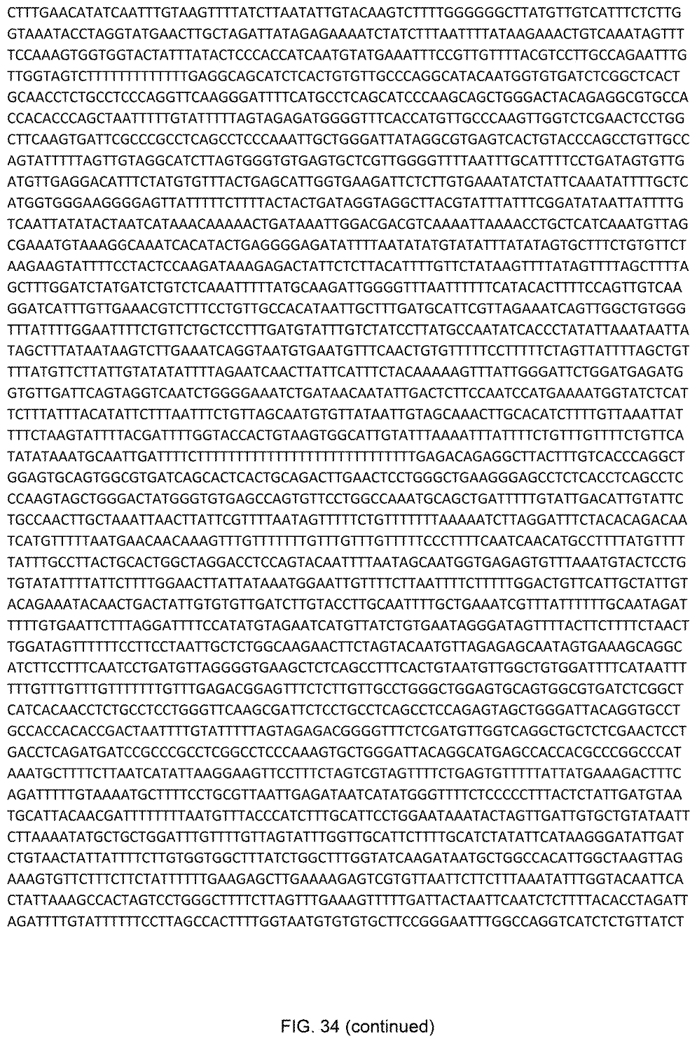

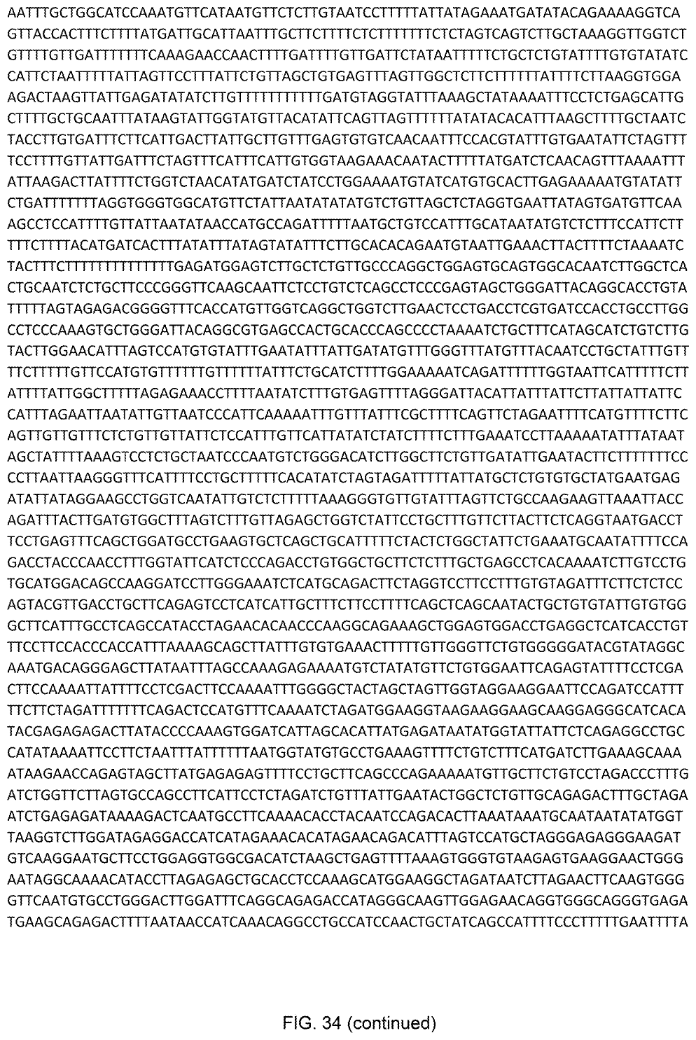

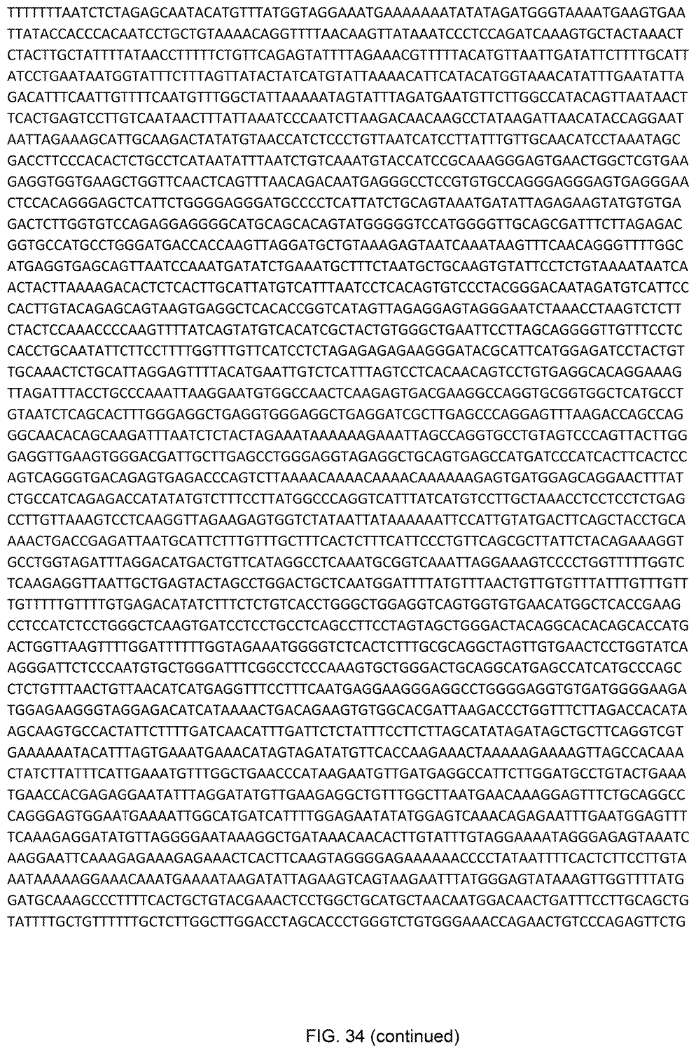

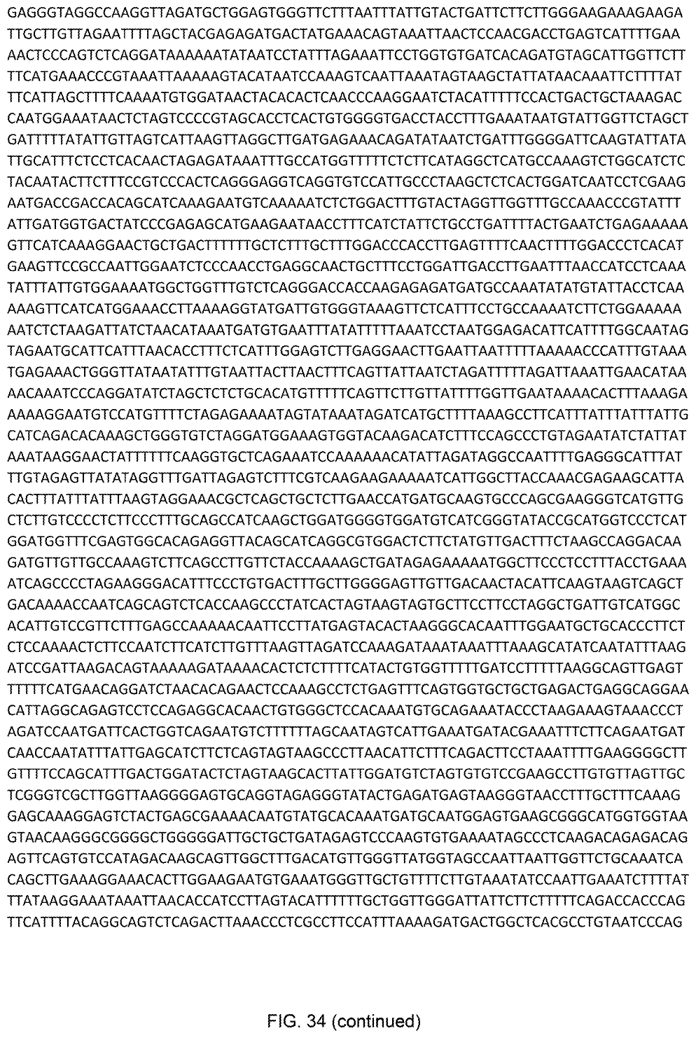

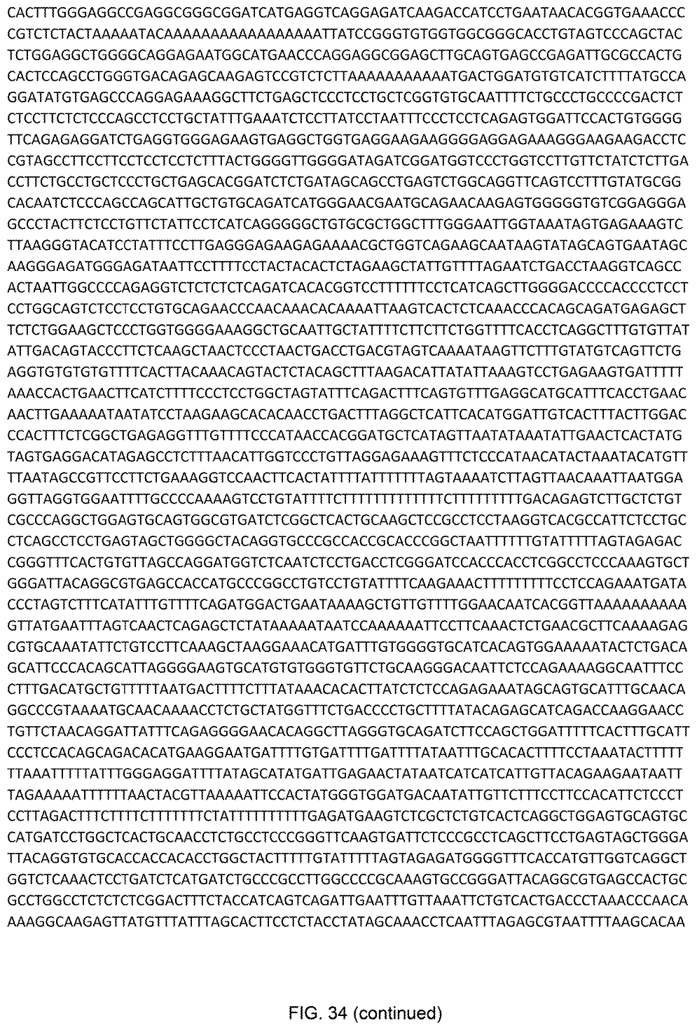

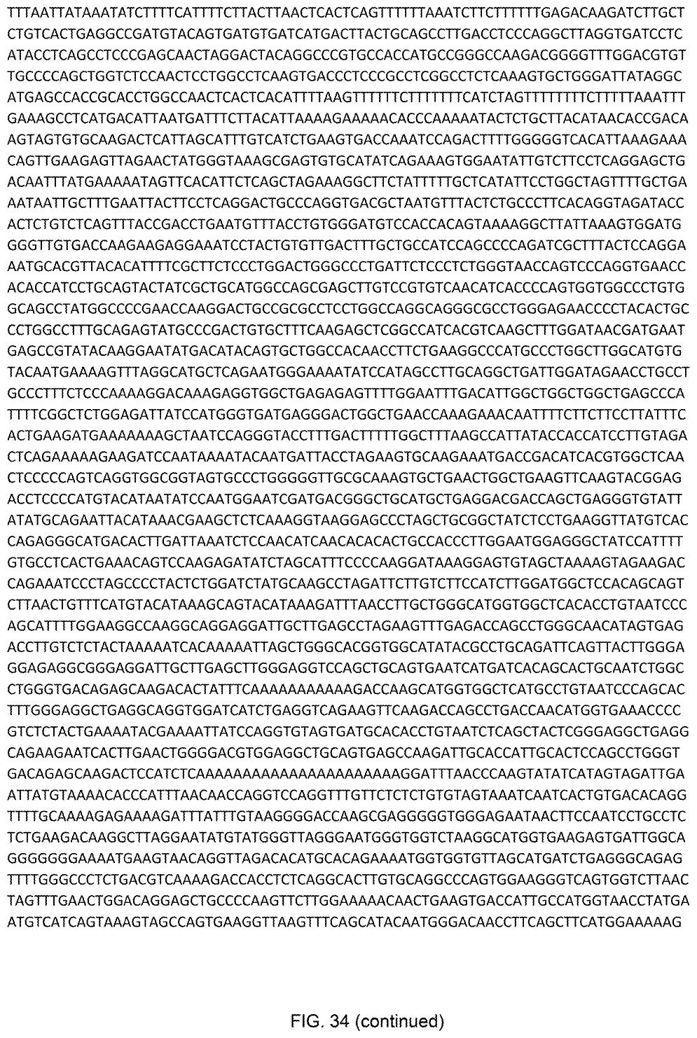

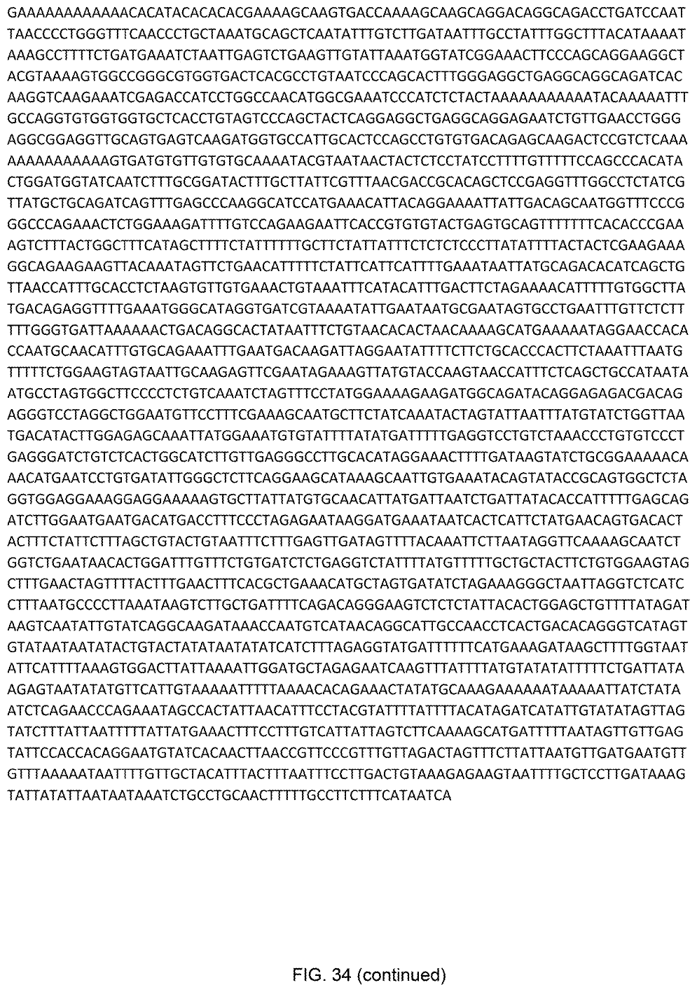

In some cases, a gene editing system designed to alter or eliminate one or more methylation sites (e.g., a 10 to 50 base pair region containing multiple CpG sites, a 10 to 100 base pair region containing multiple CpG sites, a 10 to 150 base pair region containing multiple CpG sites, a 10 to 200 base pair region containing multiple CpG sites, a 25 to 50 base pair region containing multiple CpG sites, a 25 to 100 base pair region containing multiple CpG sites, a 25 to 150 base pair region containing multiple CpG sites, a 25 to 200 base pair region containing multiple CpG sites, a 50 to 100 base pair region containing multiple CpG sites, a 50 to 150 base pair region containing multiple CpG sites, a 50 to 200 base pair region containing multiple CpG sites, a 100 to 150 base pair region containing multiple CpG sites, or a 100 to 200 base pair region containing multiple CpG sites) of a promoter that directs expression of one or more myokine polypeptides (e.g., one or more Klotho polypeptides) can be administered to a mammal having, or at risk for developing, an age-related impairment to treat the mammal. For example, a mammal (e.g., a human) having, or at risk for developing, an age-related impairment can be administered or can self-administer a gene editing system designed to replace one or more methylation sites of a promoter that directs expression of one or more myokine polypeptides with a promoter sequence that lacks one or more methylation sites to increase the expression level of one or more myokine polypeptides (e.g., a Klotho polypeptide) in one or more cells within that mammal (e.g., human). In some cases, the region from about −1 (with respect to the ATG start site) to about −500 (e.g., from −1 to −500, from −1 to −450, from −1 to −400, from −1 to −350, from −1 to −300, from −1 to −250, from −1 to −200, from −1 to −150, from −1 to −100, from −1 to −50, from −10 to −500, from −10 to −450, from −10 to −400, from −10 to −350, from −10 to −300, from −10 to −250, from −10 to −200, from −10 to −150, from −10 to −100, from −10 to −50, from −50 to −500, from −50 to −450, from −50 to −400, from −50 to −350, from −50 to −300, from −50 to −250, from −50 to −200, from −50 to −150, or from −50 to −100) of a promoter that directs expression of one or more myokine polypeptides (e.g., one or more Klotho polypeptides) can be removed. Examples of methylation sites (e.g., CpG dinucleotides) within a promotor that can drive expression of a human Klotho polypeptide that can be removed or replaced as described herein to increase the expression of Klotho polypeptides include, without limitation, those methylation sites within the underlined sequence (SEQ ID NO:4) of the sequence set forth in FIG. 34, those set forth in the Asuma et al. reference (FASEB J, 26(10):4264-4274 (2012), see, e.g., FIG. 2B), those set forth in the King et al. reference (Age (Dordr), 34(6):1405-1419 (2012), see, e.g., FIG. 4), those methylation sites within the −334 to −122 region of a ‘5 sequence of a Klotho gene as set forth in the Jin et al. reference (Eur Rev. Med. Pharmacol. Sci., 19:2544-2551 (2015), see, e.g., FIG. 3), those set forth in the Chen et al. reference (PLoS One, 8(11):e79856 (2013), see, e.g., FIGS. 1 and 2), those methylation sites within the 5′ regions of a Klotho gene as set forth in the Lee et al. reference (Mol. Cancer, 9:109 (2010), see, e.g., FIG. 2), those set forth in the Seo et al. reference (Anim. Cells Syst. (Seoul), 21(4):223-232 (2017), see, e.g., Table 1 and FIG. 5A), those methylation sites within the 5′ region of a Klotho gene as set forth in the Perveez et al. reference (Mol. Biol. Res. Commun., 4(4):217-224 (2015)), those methylation sites set forth in the Wehling-Henricks et al. reference (Hum. Mol. Genet., 25:2465-2482 (2016)), those methylation sites within the 5′ region of a Klotho gene as set forth in the Perveez et al. reference (Am. J. Cancer Res., 1(1):111-119 (2011), see, e.g., FIG. 3A), and those methylation sites set forth in the Xu et al. reference (Endocr. Rev., 36(2):174-193 (2015), see, e.g., FIG. 2). In some cases, a promotor region such as a region from about −1 (with respect to the ATG start site) to about −500 (e.g., from −1 to −500, from −1 to −450, from −1 to −400, from −1 to −350, from −1 to −300, from −1 to −250, from −1 to −200, from −1 to −150, from −1 to −100, from −1 to −50, from −10 to −500, from −10 to −450, from −10 to −400, from −10 to −350, from −10 to −300, from −10 to −250, from −10 to −200, from −10 to −150, from −10 to −100, from −10 to −50, from −50 to −500, from −50 to −450, from −50 to −400, from −50 to −350, from −50 to −300, from −50 to −250, from −50 to −200, from −50 to −150, or from −50 to −100) of a promoter that directs expression of one or more myokine polypeptides (e.g., one or more Klotho polypeptides) can be replaced with a heterologous promotor sequence. Any appropriate heterologous promotor sequence can be used.

A gene editing system designed to remove or replace one or more methylation sites of a promoter that directs expression of one or more myokine polypeptides can be any appropriate gene editing system. Examples of gene editing systems that can be designed to reduce or eliminate methylation of a promoter that directs expression of one or more myokine polypeptides include, without limitation, zinc finger nucleases (ZFNs), TALE nucleases (TALENs), and clustered regularly interspaced palindromic repeats (CRISPR)/Cas9 systems. When a CRISPR/Cas9 system is used to reduce or eliminate methylation of a promoter that directs expression of one or more myokine polypeptides, the Cas9 component of a CRISPR/Cas9 system can be any appropriate Cas9 (e.g., a Staphylococcus aureus Cas9 (saCas9)). The nucleic acid and/or polypeptide sequences of such genome editing molecules can be as described elsewhere (see, e.g., Mani et al., Biochemical and Biophysical Research Communications, 335:447-457 (2005); Campbell et al., Circulation Research, 113:571-587 (2013); Cong et al., Science, 339:819-823 (2013); and Ran et al., Nature, 520:186-191 (2015)).

A gene editing system designed to remove or replace one or more methylation sites of a promoter that directs expression of one or more myokine polypeptides can be designed to target a promoter that directs expression of any appropriate myokine described herein. In some cases, a gene editing system designed to remove or replace one or more methylation sites of a promoter that directs expression of one or more myokine polypeptides can target a promoter that directs expression of a Klotho polypeptide (e.g., a promoter that directs expression of an α-Klotho polypeptide such as a Klotho promoter).

Any appropriate method can be used to deliver a gene editing system (e.g., a CRISPR/Cas9 system) described herein or nucleic acid encoding a gene editing system described herein to a cell (e.g., a cell within a mammal). For example, a vector (e.g., a viral vector) can be used to deliver nucleic acid encoding a CRISPR/Cas9 system described herein to cells within a mammal (e.g. a human). In some cases, a single vector can be designed to deliver both a nucleic acid encoding the Cas9 component (e.g., an saCas9) and the targeting guide RNA of a CRISPR/Cas9 system.

In some cases, a demethylation agent that promotes expression of a myokine polypeptide (e.g., a Klotho polypeptide) can be administered to a mammal having, or at risk for developing, an age-related impairment to treat the mammal as described herein. Examples of such demethylation agents that can be administered to a mammal having, or at risk for developing, an age-related impairment to treat the mammal include, without limitation, rhein (see, e.g., Zhang et al., Kidney Int., 91(1):144 (2017)) and N-(2-chlorophenyl)-1H-indole-3-caboxamide (see, e.g., Jung et al., Oncotarget, 8(29):46745-46755 (2017)).

In some cases, treating a mammal having, or at risk for developing, an age-related impairment as described herein (e.g., by increasing the level of one or more myokine polypeptides such as Klotho polypeptides in one or more cells within the mammal) can be effective to restore the healing capacity of an aged cell (e.g., an aged skeletal muscle cell) within a mammal. For example, increasing the level one or more myokine polypeptides in one or more cells within a mammal can be effective to promote healing of an aged cell (e.g., an aged skeletal muscle cell) within a mammal after an injury (e.g., an acute injury). When an aged cell is a muscle cell having damaged myofibers, increasing the level one or more myokine polypeptides (e.g., an α-Klotho polypeptide) in one or more cells within a mammal can be effective to induce myofiber regeneration (e.g., functional myofiber regeneration) in the aged muscle cell. When an aged cell is a muscle cell having damaged myofibers, increasing the level one or more myokine polypeptides (e.g., an α-Klotho polypeptide) in one or more cells within a mammal can be effective to restore the muscle cell myofiber architecture in the aged muscle cell.

In some cases, treating a mammal having, or at risk for developing, an age-related impairment as described herein (e.g., by increasing the level of one or more myokine polypeptides (e.g., an α-Klotho polypeptide) in one or more cells within the mammal) can be effective to reduce cellular senescence. For example, increasing the level one or more myokine polypeptides (e.g., an α-Klotho polypeptide) in one or more cells within a mammal can be effective to reduce levels of one or more senescence markers (e.g., p16^{Ink4a} polypeptides, p21^{Cip1} polypeptides, p53, H2AX, and/or SAHF) in a cell within the mammal. Any appropriate method can be used to determine a level of one or more senescence markers expressed by cells within a mammal. Examples of methods that can be used to determine a level of senescence marker expression include, without limitation, western blotting techniques, ELISA, real-time PCR, immunofluorescence, and flow cytometry.

In some cases, treating a mammal having, or at risk for developing, an age-related impairment as described herein (e.g., by increasing the level of one or more myokine polypeptides (e.g., an α-Klotho polypeptide) in one or more cells within the mammal) can be effective to increase cellular bioenergetics (e.g., mitochondrial bioenergetics). For example, increasing the level one or more myokine polypeptides (e.g., an α-Klotho polypeptide) in one or more cells within a mammal can be effective to decrease mtDNA damage in a cell within the mammal. For example, increasing the level one or more myokine polypeptides (e.g., an α-Klotho polypeptide) in one or more cells within a mammal can be effective to increase the oxygen consumption rate (OCR) in a cell within the mammal. In some cases, increasing the level one or more myokine polypeptides (e.g., an α-Klotho polypeptide) in one or more cells within a mammal can be effective to increase the reserve capacity in a cell within the mammal.

In some cases, treating a mammal having, or at risk for developing, an age-related impairment as described herein (e.g., by increasing the level of one or more myokine polypeptides (e.g., an α-Klotho polypeptide) in one or more cells within the mammal) can be effective to induce cellular division, to reduce fibrosis, and/or to enhance muscle progenitor cell (MPC) lineage progression.

This document also provides compositions containing (a) one or more myokine polypeptides (e.g., an α-Klotho polypeptide), (b) nucleic acid encoding one or more myokine polypeptides (e.g., a nucleic acid vector encoding an α-Klotho polypeptide), (c) exosomes containing (i) one or more myokine polypeptides (e.g., one or more Klotho polypeptides) and/or (ii) nucleic acid encoding one or more myokine polypeptides (e.g., one or more Klotho polypeptides), and/or (d) a gene editing system designed to reduce or eliminate methylation of a promoter that directs expression of a myokine polypeptide (e.g., an α-Klotho polypeptide). For example, one or more myokine polypeptides (e.g., an α-Klotho polypeptide), nucleic acid encoding one or more myokine polypeptides (e.g., a nucleic acid vector encoding an α-Klotho polypeptide), one or more exosomes provided herein, and/or a gene editing system designed to reduce or eliminate methylation of a promoter that directs expression of a myokine polypeptide (e.g., an α-Klotho polypeptide) can be formulated into a composition (e.g., a pharmaceutically acceptable composition) for administration to a mammal having, or at risk for developing, an age-related impairment. For example, one or more myokine polypeptides (e.g., an α-Klotho polypeptide), nucleic acid encoding one or more myokine polypeptides (e.g., a nucleic acid vector encoding an α-Klotho polypeptide), one or more exosomes provided herein, and/or a gene editing system designed to reduce or eliminate methylation of a promoter that directs expression of a myokine polypeptide (e.g., an α-Klotho polypeptide) can be formulated together with one or more pharmaceutically acceptable carriers (additives) and/or diluents. Pharmaceutically acceptable carriers, fillers, and vehicles that may be used in a pharmaceutical composition described herein include, without limitation, saline, dimethyl sulfoxide (DMSO), ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, and wool fat.

A composition including one or more myokine polypeptides (e.g., an α-Klotho polypeptide), nucleic acid encoding one or more myokine polypeptides (e.g., a nucleic acid vector encoding an α-Klotho polypeptide), one or more exosomes provided herein, and/or a gene editing system designed to reduce or eliminate methylation of a promoter that directs expression of a myokine polypeptide (e.g., an α-Klotho polypeptide) can be designed for oral or parenteral (including intraperitoneal, subcutaneous, intramuscular, intravenous, and intradermal) administration to a mammal (e.g., a human) having, or at risk for developing, an age-related impairment. Compositions suitable for oral administration include, without limitation, liquids, tablets, capsules, pills, powders, gels, and granules. Compositions suitable for parenteral administration include, without limitation, aqueous and non-aqueous sterile injection solutions that can contain anti-oxidants, buffers, bacteriostats, and solutes that render the formulation isotonic with the blood of the intended recipient. In some cases, a composition including one or more myokines and/or nucleic acid encoding one or more myokines can be formulated for intraperitoenal administration (e.g., intraperitoneal injection).

A composition including one or more myokine polypeptides (e.g., an α-Klotho polypeptide), nucleic acid encoding one or more myokine polypeptides (e.g., a nucleic acid vector encoding an α-Klotho polypeptide), one or more exosomes provided herein, and/or a gene editing system designed to reduce or eliminate methylation of a promoter that directs expression of a myokine polypeptide (e.g., an α-Klotho polypeptide) can be designed for any type of release (e.g., release of the one or more myokine polypeptides, nucleic acid encoding one or more myokine polypeptides, and/or a gene editing system designed to reduce or eliminate methylation of a promoter that directs expression of a myokine polypeptide from the composition) into the mammal that the composition is administered to (e.g., a mammal having, or at risk for developing, an age-related impairment). For example, a composition including one or more myokine polypeptides (e.g., an α-Klotho polypeptide), nucleic acid encoding one or more myokine polypeptides (e.g., a nucleic acid vector encoding an α-Klotho polypeptide), one or more exosomes provided herein, and/or a gene editing system designed to reduce or eliminate methylation of a promoter that directs expression of a myokine polypeptide (e.g., an α-Klotho polypeptide) can be designed for immediate release, slow release, or extended release of the myokine polypeptide, nucleic acid, or gene editing system.

A composition including one or more myokine polypeptides (e.g., an α-Klotho polypeptide), nucleic acid encoding one or more myokine polypeptides (e.g., a nucleic acid vector encoding an α-Klotho polypeptide), one or more exosomes provided herein, and/or a gene editing system designed to reduce or eliminate methylation of a promoter that directs expression of a myokine polypeptide (e.g., an α-Klotho polypeptide) can be administered locally or systemically to a mammal (e.g., a human) having, or at risk for developing, an age-related impairment. For example, a composition including one or more myokine polypeptides (e.g., an α-Klotho polypeptide), nucleic acid encoding one or more myokine polypeptides (e.g., a nucleic acid vector encoding an α-Klotho polypeptide), one or more exosomes provided herein, and/or a gene editing system designed to reduce or eliminate methylation of a promoter that directs expression of a myokine polypeptide (e.g., an α-Klotho polypeptide) can be administered systemically by intraperitoneal administration to a mammal having, or at risk for developing, an age-related impairment.

A composition including one or more myokine polypeptides (e.g., an α-Klotho polypeptide), nucleic acid encoding one or more myokine polypeptides (e.g., a nucleic acid vector encoding an α-Klotho polypeptide), one or more exosomes provided herein, and/or a gene editing system designed to reduce or eliminate methylation of a promoter that directs expression of a myokine polypeptide (e.g., an α-Klotho polypeptide) can be administered to a mammal (e.g., a human) having, or at risk for developing, an age-related impairment in any appropriate dose(s). Effective doses can vary depending on the severity of the age-related impairment, the risk for developing an age-related impairment, the route of administration, the age and general health condition of the subject, excipient usage, the possibility of co-usage with other therapeutic treatments such as use of other agents, and the judgment of the treating physician. In cases where a composition includes one or more myokine polypeptides (e.g., an α-Klotho polypeptide), an effective dose of that composition can be from about 5 picograms of myokine polypeptides per milliliter (pg/mL) liquid (e.g., saline) to about 6 μg/mL (e.g., from about 5 pg/mL to about 5 μg/mL, from about 5 pg/mL to about 5 μg/mL, from about 5 pg/mL to about 1 μg/mL, from about 5 pg/mL to about 0.5 μg/mL, from about 5 pg/mL to about 0.1 μg/mL, from about 5 pg/mL to about 0.05 μg/mL, from about 50 pg/mL to about 1 μg/mL, from about 500 pg/mL to about 1 μg/mL, from about 1 ng/mL to about 1 μg/mL, or from about 100 ng/mL to about 500 ng/mL). In some cases, a composition including a Klotho polypeptide (e.g., α-Klotho polypeptide) can be from about 100 pg/mL to about 500 pg/mL (e.g., about 324 pg/mL). In some cases where a composition includes an α-Klotho polypeptide, the composition can be administered to deliver from about 0.001₁1g to about 500 μg (e.g., from about 0.01 μg to about 500 μg, from about 0.05 μg to about 500 μg, from about 0.1 μg to about 500 μg, from about 1 μg to about 500 μg, from about 10 μg to about 500 μg, from about 100 μg to about 500 μg, from about 0.001 μg to about 250 μg, from about 0.001 μg to about 100 μg, from about 0.001 μg to about 50 μg, from about 0.001 μg to about 5 μg, from about 0.1 μg to about 250 μg, from about 1 μg to about 100 μg, from about 5 μg to about 50 μg, from about 10 μg to about 50 λg, or from about 10 μg to about 30 μg) of an α-Klotho polypeptide per kg body weight of a mammal (e.g., a human).

An effective amount of a composition including one or more myokine polypeptides (e.g., an α-Klotho polypeptide), nucleic acid encoding one or more myokine polypeptides (e.g., an α-Klotho polypeptide), one or more exosomes provided herein, and/or a gene editing system designed to reduce or eliminate methylation of a promoter that directs expression of a myokine polypeptide can be any amount that reduces the severity and/or one or more symptom of a condition being treated (e.g., an age-related impairment) without producing significant toxicity to the mammal. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, severity of the age-related impairment, and risk for developing an age-related impairment may require an increase or decrease in the actual effective amount administered.

A composition including one or more myokine polypeptides (e.g., an α-Klotho polypeptide), nucleic acid encoding one or more myokine polypeptides (e.g., a nucleic acid vector encoding an α-Klotho polypeptide), one or more exosomes provided herein, and/or a gene editing system designed to reduce or eliminate methylation of a promoter that directs expression of a myokine polypeptide (e.g., an α-Klotho polypeptide) can be administered to a mammal (e.g., a human) having, or at risk for developing, an age-related impairment in any appropriate frequency. The frequency of administration can be any frequency that reduces the severity of the age-related impairment and/or one or more symptoms of the age-related impairment without producing significant toxicity to the mammal. For example, the frequency of administration can be from about every three days to about ten times a day, from about every other day to about five times a day, or from about one time a day to about two times a day. In some cases, the frequency of administration can be once a day. The frequency of administration can remain constant or can be variable during the duration of treatment. As with the effective amount, various factors can influence the actual frequency of administration used for a particular application. For example, the effective amount, duration of treatment, use of multiple treatment agents, route of administration, severity of the age-related impairment, and risk for developing an age-related impairment may require an increase or decrease in administration frequency.

A composition including one or more myokine polypeptides (e.g., an α-Klotho polypeptide), nucleic acid encoding one or more myokine polypeptides (e.g., a nucleic acid vector encoding an α-Klotho polypeptide), one or more exosomes provided herein, and/or a gene editing system designed to reduce or eliminate methylation of a promoter that directs expression of a myokine polypeptide (e.g., an α-Klotho polypeptide) can be administered to a mammal (e.g., a human) having, or at risk for developing, an age-related impairment for any appropriate duration. An effective duration for administering a composition including one or more myokine polypeptides (e.g., an α-Klotho polypeptide), nucleic acid encoding one or more myokine polypeptides (e.g., a nucleic acid vector encoding an α-Klotho polypeptide), one or more exosomes provided herein, and/or a gene editing system designed to reduce or eliminate methylation of a promoter that directs expression of a myokine polypeptide (e.g., an α-Klotho polypeptide) can be any duration that reduces the severity of the age-related impairment and/or one or more symptoms of the age-related impairment without producing significant toxicity to the mammal. For example, the effective duration can vary from several days to several months or years to a lifetime. In some cases, the effective duration for the treatment of an age-related impairment can range in duration from about 2 days to about a week. Multiple factors can influence the actual effective duration used for a particular treatment. For example, an effective duration can vary with the frequency of administration, effective amount, use of multiple treatment agents, route of administration, severity of the age-related impairment, and risk for developing an age-related impairment.

In some cases, one or more myokine polypeptides (e.g., an α-Klotho polypeptide), nucleic acid encoding one or more myokine polypeptides (e.g., a nucleic acid encoding an α-Klotho polypeptide), one or more exosomes provided herein, and/or a gene editing system designed to reduce or eliminate methylation of a promoter that directs expression of a myokine polypeptide (e.g., an α-Klotho polypeptide) can be administered to a mammal having, or at risk for developing, an age-related impairment as the sole active ingredient. For example, one or more myokine polypeptides (e.g., an α-Klotho polypeptide), nucleic acid encoding one or more myokine polypeptides (e.g., a nucleic acid encoding an α-Klotho polypeptide), one or more exosomes provided herein, and/or a gene editing system designed to reduce or eliminate methylation of a promoter that directs expression of a myokine polypeptide (e.g., an α-Klotho polypeptide) can be administered to a mammal having, or at risk for developing, an age-related impairment as the sole active ingredient used to treat an age-related impairment. In some cases, one or more myokine polypeptides (e.g., an α-Klotho polypeptide) or nucleic acid encoding one or more myokine polypeptides (e.g., a nucleic acid encoding an α-Klotho polypeptide) can be administered as the sole active ingredient to a mammal in need thereof (e.g., a mammal such as a human having, or at risk for developing, an age-related impairment).

In some cases, one or more myokine polypeptides (e.g., an α-Klotho polypeptide), nucleic acid encoding one or more myokine polypeptides (e.g., a nucleic acid encoding an α-Klotho polypeptide), one or more exosomes provided herein, and/or a gene editing system designed to reduce or eliminate methylation of a promoter that directs expression of a myokine polypeptide (e.g., an α-Klotho polypeptide) can be administered to a mammal having, or at risk for developing, an age-related impairment with one or more additional agents and/or therapies. For example, one or more myokine polypeptides (e.g., an α-Klotho polypeptide) and/or nucleic acid encoding one or more myokine polypeptides (e.g., a nucleic acid encoding an α-Klotho polypeptide) can be administered to a mammal having, or at risk for developing, an age-related impairment with one or more additional agents or therapies used to treat an age-related impairment. Examples of additional agents or therapies used to treat an age-related impairment include, without limitation, senolytics, metformin, and rapamycin. In cases where one or more myokine polypeptides (e.g., an α-Klotho polypeptide), nucleic acid encoding one or more myokine polypeptides (e.g., a nucleic acid encoding an α-Klotho polypeptide), and/or a gene editing system designed to reduce or eliminate methylation of a promoter that directs expression of a myokine polypeptide (e.g., an α-Klotho polypeptide) are used in combination with one or more additional agents or therapies, the one or more additional agents or therapies can be administered at the same time or independently. For example, a composition including one or more myokine polypeptides (e.g., an α-Klotho polypeptide), nucleic acid encoding one or more myokine polypeptides (e.g., a nucleic acid encoding an α-Klotho polypeptide), and/or a gene editing system designed to reduce or eliminate methylation of a promoter that directs expression of a myokine polypeptide (e.g., an α-Klotho polypeptide) can be administered first, and the one or more additional agents or therapies can be administered second, or vice versa.

In certain instances, a course of treatment and the severity of one or more symptoms related to the condition being treated (e.g., an age-related impairment) can be monitored. Any appropriate method can be used to determine whether or not the severity of a symptom is reduced. For example, the severity of an age-related impairment can be assessed using any appropriate methods and/or techniques and can be assessed at different time points. For example, muscle strength, muscle endurance, and/or fine motor control can be assessed to determine the severity of an age-related impairment.

The invention will be further described in the following examples, which do not limit the scope of the invention described in the claims.

### EXAMPLES

### Example 1: Age-Related Declines in α-Klotho Drive Progenitor Cell Mitochondrial Dysfunction and Impaired Muscle Regeneration

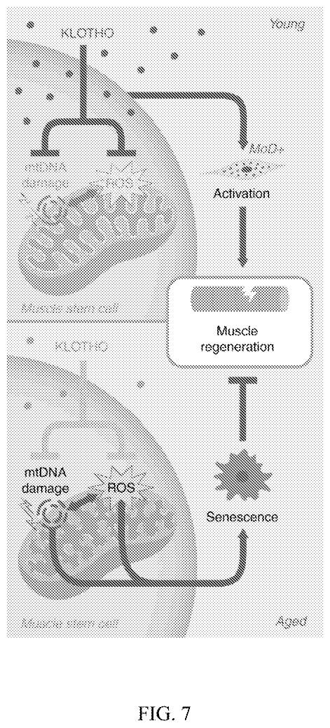

While young muscle is capable of restoring the original architecture of damaged myofibers, aged muscle displays a markedly reduced regeneration. This example shows that expression of the “anti-aging” protein, α-Klotho, is up-regulated within young injured muscle as a result of transient Klotho promoter demethylation. However, epigenetic control of the Klotho promoter is lost with aging. Genetic inhibition of α-Klotho in vivo disrupts muscle progenitor cell (MPC) lineage progression and impairs myofiber regeneration, revealing a critical role for α-Klotho in the regenerative cascade. Genetic silencing of Klotho in young MPCs drives mitochondrial DNA (mtDNA) damage and decreased cellular bioenergetics. Conversely, supplementation with α-Klotho restores mtDNA integrity and bioenergetics of aged MPCs to youthful levels in vitro and enhances functional regeneration of aged muscle in vivo in a temporally-dependent manner. These studies identify a role for α-Klotho in the regulation of MPC mitochondrial function and implicate α-Klotho declines as a driver of impaired muscle regeneration with age (FIG. 7).

### Results

α-Klotho is Highly Expressed in Acutely Injured Skeletal Muscle and MPCs of Young Animals, but Expression is Decreased with Aging

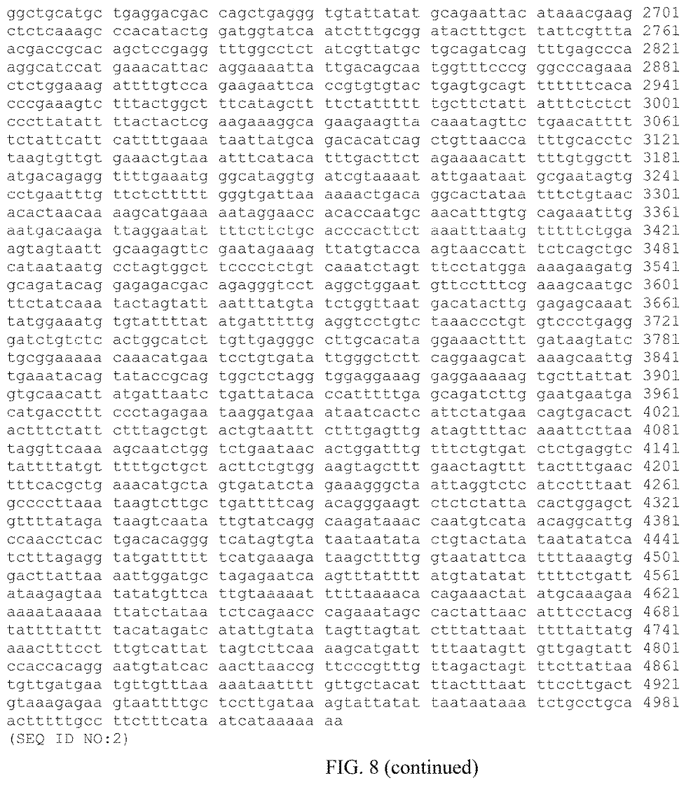

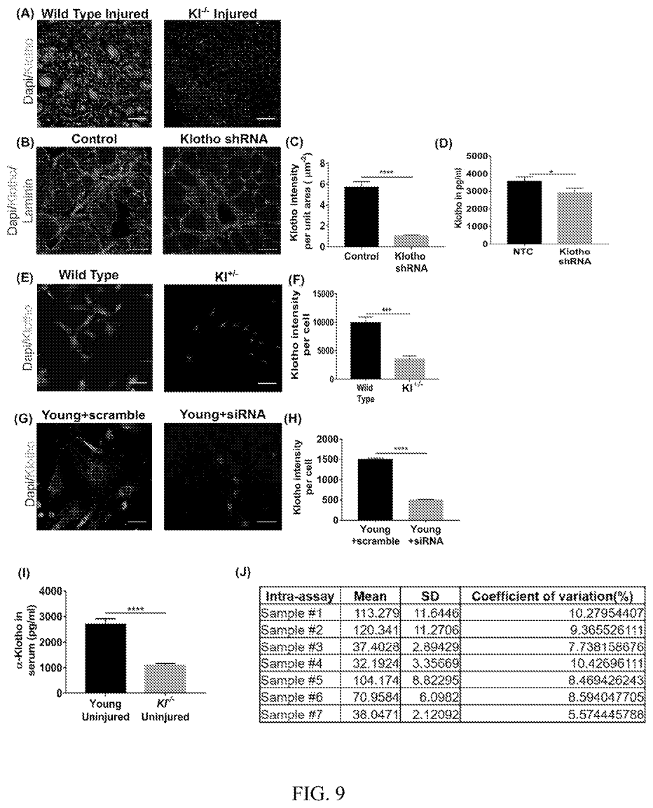

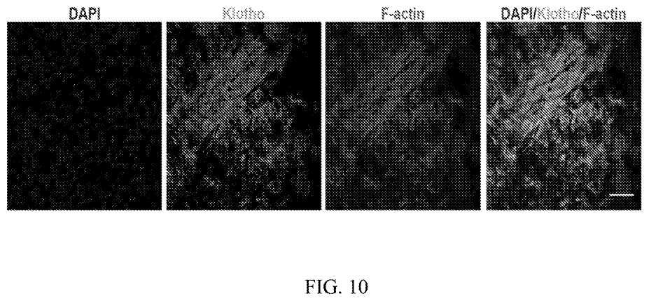

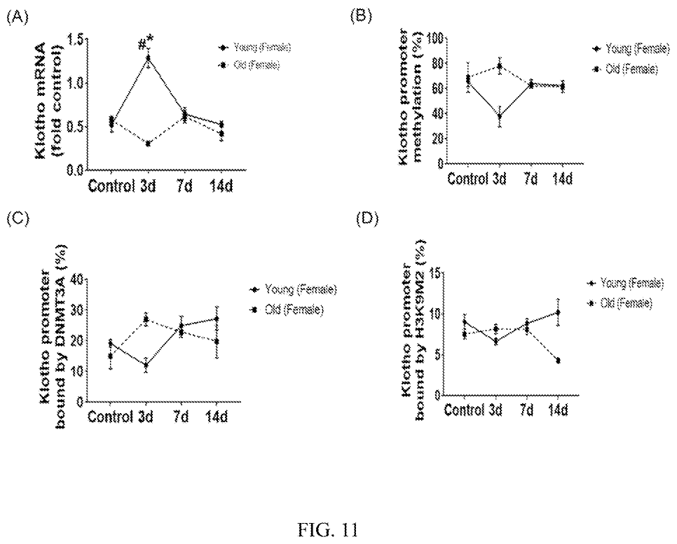

To determine whether α-Klotho is up-regulated locally in response to an acute muscle injury, immunofluorescence analysis of α-Klotho in the skeletal muscle was performed in young (4-6 months) and aged (22-24 months) male mice under conditions of homeostasis and following a cardiotoxin-induced injury. α-Klotho was virtually undetectable in healthy, uninjured muscle, regardless of age (FIG. 1A, B, E). In contrast, strong expression of α-Klotho was observed at the regenerating site of young muscle 14 days post injury (FIG. 1C, E; confirmation of antibody specificity is presented in FIG. 9). Aged muscle, however, displayed no appreciable increase in α-Klotho expression following an acute injury (FIG. 1D, E). Serum α-Klotho levels followed a similar expression pattern according to age and injury status (FIG. 1F). RT-qPCR findings revealed that Klotho transcript expression increases significantly at three and seven days post-injury in the skeletal muscle of young male mice (FIG. 1G). Despite the fact that α-Klotho protein is still detected in young muscle at 14 days post-injury (FIG. 1C, E), gene expression approached baseline levels at this later time point. On the other hand, aged counterparts display unaltered gene expression across all the time points tested (FIG. 1G). Young mice exposed to a severe contusion injury displayed a robust α-Klotho response at the protein level 14 days after injury (FIG. 10). Young female mice displayed a similar, yet blunted, increase in Klotho expression in response to injury, but the response is lost with aging (FIG. 11).

Epigenetic silencing of the α-Klotho gene contributes to the impaired regenerative potential of dystrophic skeletal muscle, and a differentially methylated region (DMR) of 110 nucleotides within the Klotho promoter region was identified in the muscles of aged mdx mice (Wehling-Henricks et al., 2016 Hum Mol Genet, 25:2465-2482). Therefore, methylation levels of the DMR after injury were measured in young and aged muscle of mice. An acute injury to young muscle triggered demethylation of the DMR in the Klotho promoter three and seven days after injury (FIG. 1H). Injury-induced demethylation was, however, absent in the Klotho promoter within aged muscle (FIG. 1H).

To examine whether modifying enzymes for DNA methylation contribute to methylation changes in the Kl promoter, a chromatin immunoprecipitation (ChIP) assay was used to measure the enrichment of DNMT3a methyltransferases in the Klotho promoter region (see, e.g., Wehling-Henricks et al., 2016 Hum Mol Genet, 25:2465-2482). There was a decrease of DNMT3a binding to the Klotho promoter in the injured young muscle that, at the time points evaluated, reached a nadir three days after injury, and was increased by day 14 (FIG. 1I). In contrast, the injury-induced decrease in DNMT3a binding was delayed and blunted in injured aged muscle (FIG. 1I). H3K9me2 binding to the Klotho promoter region was decreased after injury in young muscle (FIG. 1J). Taken together, these findings suggest that acute injury drives the reactivation of Klotho by reducing DNA methylation and H3K9 dimethylation in the promoter of young muscle, but that Klotho remains epigenetically repressed after injury in aged muscle.

### Genetic Inhibition of α-Klotho Impairs Skeletal Muscle Regeneration

To directly implicate a functional role for α-Klotho in skeletal muscle regeneration, the regenerative response to acute muscle injury in adult mice that are heterozygously deficient for Klotho (Kl+/− mice) was evaluated. Kl+/− mice displayed a significantly decreased local α-Klotho expression at the site of injury (FIG. 2A, B). Kl+/− mice also displayed a decreased regenerative index, smaller myofiber cross-sectional area, and increased fibrosis when compared to age- and sex-matched wild type counterparts (FIG. 2A-E). These findings are consistent with a recent study of muscle regeneration in α-Klotho hypomorphs (Ahrens et al., 2018). Together, the data demonstrate that α-Klotho is necessary for effective skeletal muscle regeneration after injury.

Given our findings of a significantly increased Klotho expression within the injured muscles of young mice (FIG. 1C, E, G), the contribution of local α-Klotho in functional muscle regeneration was next evaluated. The tibialis anterior (TA) muscles of young mice were injected with GFP-tagged SMARTpool® lentiviral particles carrying shRNA to α-Klotho, whereas control counterparts were injected with an equal volume of the non-targeting control (NTC) lentivirus. After four weeks, TAs were injured via a local cardiotoxin injection, and regeneration was evaluated two weeks after injury. Muscles treated with shRNA to α-Klotho displayed a significant decrease in α-Klotho expression at the site of injury (FIG. 9B). Circulating α-Klotho was also significantly decreased (FIG. 9D). This suggests that muscle-derived α-Klotho may contribute to the increased circulating levels observed in young mice after injury (FIG. 1F).

Histological analysis revealed that knock down of α-Klotho expression resulted in a decreased number of regenerating fibers, a decrease in the percentage of myofiber area/total area, and an increased adiposity (FIG. 2F-I). There was, however, no difference in the fibrosis across groups (FIG. 2K). The cross-sectional area of regenerating (centrally-nucleated) fibers in muscles treated with shRNA to α-Klotho was also significantly smaller than NTC controls (FIG. 2L), further confirming a defective regenerative response. Unexpectedly, the presence of a number of large, nonregenerating myofibers were also observed at the injury site of muscles treated with shRNA to α-Klotho (FIG. 2F). To evaluate the structural integrity of these myofibers, second harmonic generation (SHG) imaging was performed, which allows for 3-dimensional visualization of myofiber structure and organization. Consistent with histological findings, SHG analysis revealed that muscles treated with shRNA to α-Klotho contain a decreased number of centrally-nucleated fibers (FIG. 2M, N). This decreased evidence of active regeneration was concomitant with pathologic myofiber architecture and integrity (FIG. 2M). The impaired regenerative response and disrupted myofiber structure was concomitant with a decreased functional recovery after injury (FIG. 20).

### α-Klotho is Expressed by MuSCs and Their Progeny

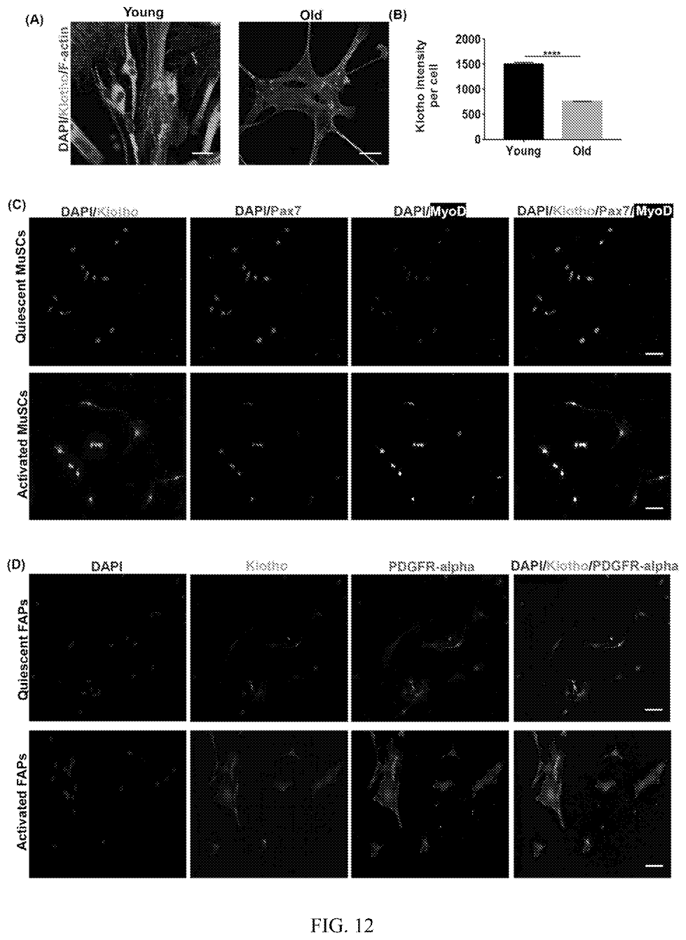

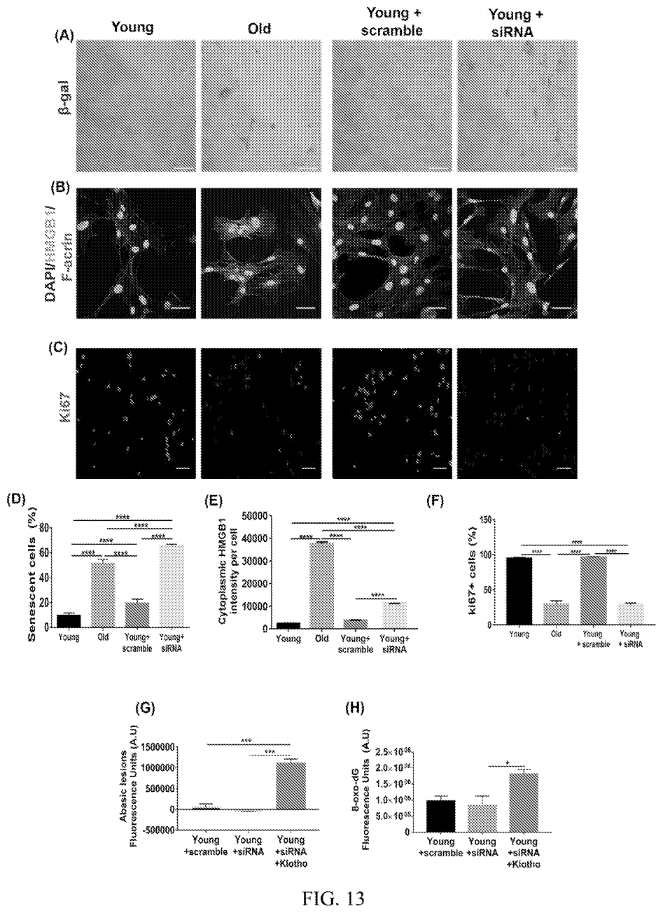

α-Klotho expression in young muscle is elevated at 3 days after injury (FIG. 1G)—a time point that corresponds with MuSC activation, so whether MuSCs express α-Klotho and whether α-Klotho is necessary for the MuSC response to injury was investigated. RNAseq data was accessed from a recent study (van Velthoven et al., 2017 Cell Rep, 21:1994-2004), which is stored on the Gene Expression Omnibus (GEO) publicly accessible database. Analysis of archived data revealed a 10-fold increase in Klotho expression of freshly sorted MuSCs as compared to whole muscle lysates (FIG. 3A). Structured illumination microscopy (SIM) confirmed robust α-Klotho in MPCs isolated from young mice (FIG. 3B, C). MPCs were cultured for no more than three passages prior to analysis and were confirmed to be >90% MyoD+. MPCs isolated from aged muscle, however, displayed a markedly decreased α-Klotho protein expression (FIG. 3B, C). α-Klotho expression in MuSCs isolated was also evaluated by fluorescence activated cell sorting. As observed in MPCs, it was found that young MuSCs displayed a robust α-Klotho expression, but that α-Klotho expression was decreased in aged MuSCs (FIG. 12).

To determine whether MPCs secrete α-Klotho, ELISA of the conditioned media from MPCs isolated from young and old muscle was performed. Conditioned media derived from young MPCs contained significantly more α-Klotho than the conditioned media obtained from aged MPCs (FIG. 3D), suggesting that there is an age-related declines in the capacity of MPCs to secrete α-Klotho.

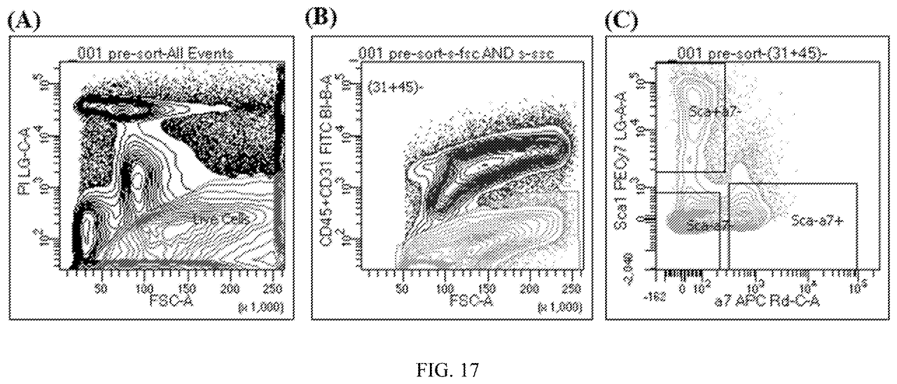

Immunohistochemical analysis of young muscle three days after injury revealed that 96.7% of MyoD+cells express α-Klotho (FIG. 3E). MuSCs RNAseq data housed on the GEO repository (van Velthoven et al., 2017 Cell Rep, 21:1994-2004) were accessed and analyzed and it was found that activated MuSCs display significantly increased Klotho expression when compared to quiescent MuSC counterparts that were isolated from 1% PFA-perfused mice (FIG. 3F). These findings suggest that α-Klotho expression is increased in the transition from MuSC quiescence to activation. To investigate this more directly, α-Klotho expression in freshly sorted (quiescent) MuSCs and MuSCs that were maintained in culture for three days was compared, thereby promoting activation in vitro (see gating strategy in FIG. 17). Activated MuSCs displayed a 4.5-fold increase in α-Klotho, when compared to quiescent counterparts (FIG. 3G, H). Conditioned media derived from activated MuSCs also contained significantly more α-Klotho, when compared to conditioned media derived from quiescent MuSCs (FIG. 3I). MuSCs from uninjured young muscle and young muscle three days post-injury were also isolated, yielding a population of quiescent and activated MuSCs, respectively (FIG. 12C). As observed when MuSCs were activated in vitro, there was a significant increase in α-Klotho expression in MuSCs that were activated in vivo (FIG. 3J, K). As a comparison, fibroadipogenic progenitor cells (FAPs), which also play a critical role in the skeletal muscle regenerative cascade, displayed no change in α-Klotho over three days of activation in culture, nor was α-Klotho expression increased when FAPS were isolated from acutely injured muscle (FIG. 3 G, H, J, K, FIG. 12D). The conditioned media from activated FAPs also contained significantly less α-Klotho when compared to the conditioned media from activated MuSCs (FIG. 3I). Therefore, at least some of the α-Klotho protein detected in muscle after injury could come from MPCs themselves, although other neighboring cell populations may also express and secrete α-Klotho in response to an acute injury.

It was next asked whether α-Klotho is necessary for normal MuSC lineage progression. MPCs isolated from the skeletal muscle of Kl+/− mice displayed a small, but significant, decrease in the percentage of MyoD+cells when compared to age-matched wild type counterparts. There was, however, no difference in Pax7 expression across groups (FIG. 3L-N). In vivo, lentiviral shRNA inhibition of α-Klotho resulted in a decreased MyoD expression at the site of injury (FIG. 3O, P). Taken together, these data suggest that a loss of α-Klotho disrupts MuSC lineage progression.

### Loss of α-Klotho Expression in MPCs Drives Cellular Senescence and Mitochondrial Dysfunction

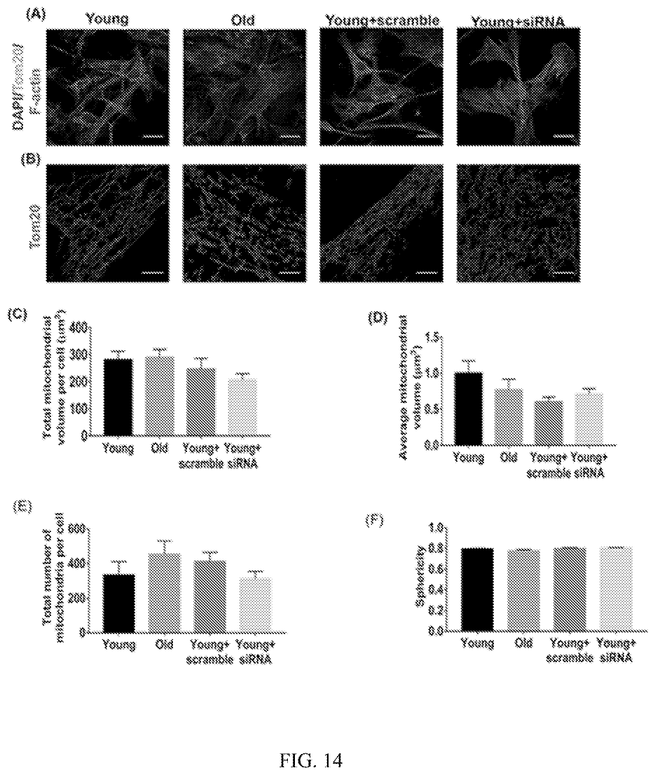

It was investigated whether the decreased MuSC activation in α-Klotho-deficient muscles may be attributed to increased cellular senescence. To confirm an inhibitory role for α-Klotho in cellular senescence in myogenic cells, small interference RNA (siRNA) inhibition was used, which resulted in a ˜3-fold decrease in α-Klotho (FIG. 9G, H). As expected, knockdown of α-Klotho induced a senescent phenotype in young MPCs, as evidenced by the percentage of senescence associated (SA)-βgal positive cells, increased cytosolic HMGB1 levels (an indicator of cellular stress) and decreased cellular proliferation (FIG. 13A, B, D, E). These findings mimicked the phenotype of MPCs isolated from aged muscle (FIG. 13).

LXRepair multiplex technology (see, e.g., Garreau-Balandier et al., 2014 FEBS Lett, 588:1673-9; and Sauvaigo et al., 2004 Anal Biochem, 333:182-92) was used to evaluate the DNA base excision repair (BER) enzyme activities of OGG1 and APE1, which work on two common oxidative DNA lesions, 8-oxodG and abasic sites, respectively. When compared to scramble siRNA-treated young MPCs, there was no significant decline in base excision repair (BER) in the nucleus of cells treated with siRNA to α-Klotho (FIG. 13G, H). However, just 48 hours of α-Klotho supplementation to siRNA-treated MPCs from young animals dramatically stimulated the activity of these BER enzymes (FIG. 13G, H). These data suggest that α-Klotho's role in MPC senescence may be attributed to induction of key enzymes in the BER pathway, which is rapid and can occur even within a couple of hours (Chen et al., 1998).

While α-Klotho's role in cellular senescence has been demonstrated in multiple systems, the mechanisms underlying this role are incompletely understood. The possibility that α-Klotho may regulate MPC mitochondrial structure and function was evaluated. Using an antibody against the mitochondrial membrane protein, Tom20, no difference in mitochondrial morphology was observed, as determined by sphericity, the number of mitochondria per cell, or mitochondrial volume according to age or α-Klotho levels (FIG. 14A, C-F). This was further supported qualitatively using STimulation Emission Depletion (STED) microscopy to visualize the mitochondrial network (FIG. 14B). These findings suggest that a loss of α-Klotho does not affect mitochondrial morphology or mass. However, detailed analysis by transmission electron microscopy (TEM) revealed a striking alteration of ultrastructural integrity of the mitochondrial cristae and endoplasmic reticulum, in addition to lipid droplet accumulation when young MPCs were treated with siRNA to α-Klotho (FIG. 4A).

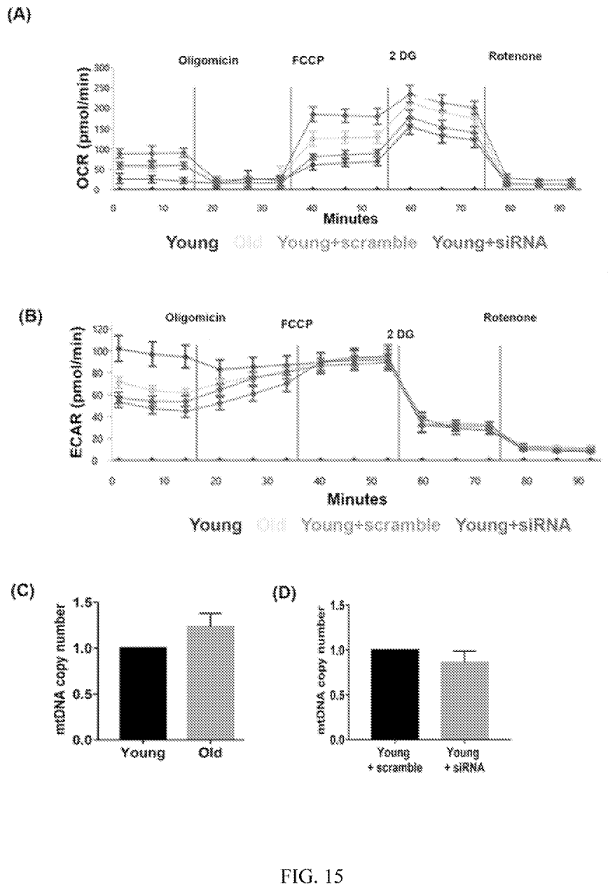

In light of the loss of mitochondrial ultrastructural resulting from decreased α-Klotho expression, we next evaluated whether loss of α-Klotho drives MPC mitochondrial dysfunction. The bioenergetic profiles of young and aged MPCs were studied using a Seahorse XFe96 Flux analyzer, which measures oxygen consumption rate (OCR), a measure of oxidative phosphorylation. OCR was measured again after injection of oligomycin, FCCP, 2-deoxyglucose (2-DG), and rotenone. These data demonstrate that, when normalized to total number of cells, MPCs from older animals display dramatically decreased levels of basal OCR as compared to young counterparts (FIG. 4B; FIG. 15). When young MPCs were treated with siRNA to α-Klotho, basal OCR was reduced to ˜25% values of scramble-treated counterparts (FIG. 4C, FIG. 14). MPCs isolated from uninjured Kl+/− mice display a similarly blunted bioenergetic profile (FIG. 4D). Though there was no appreciable decrease in reserve capacity in aged MPCs, young MPCs treated with siRNA to α-Klotho and MPCs isolated from Kl+/− mice both showed a decrease of ˜25% in the reserve capacity (FIG. 4E-G; FIG. 15). Reserve capacity represents the spare bioenergetic capacity, is calculated as the difference between the basal and maximal OCR, and indicates the ability of a cell to respond to stress. Taken together, these data support the hypothesis that age-related declines in α-Klotho drive impaired MPC mitochondrial bioenergetics.

mtDNA integrity was next examined in MPCs isolated from young or aged mice, using a qPCR-based assay. The method used is based on the principle that a wide variety of types of DNA damage have the propensity to block DNA polymerase progression (see, e.g., Furda et al., 2012 DNA Repair (Amst), 11:684-92). Therefore, this assay detects numerous kinds of base DNA damage or DNA repair intermediates such as abasic sites, as well as single and double DNA strand breaks.

There was no difference in steady-state mtDNA copy number across groups (FIG. 15C, D), consistent with immunocytochemical analysis showing no difference in mitochondrial mass (FIG. 14C). However, it was found that MPCs isolated from aged mice displayed higher levels of mtDNA damage as compared to young counterparts (FIG. 4H). Accordingly, young MPCs receiving siRNA to α-Klotho and MPCs isolated from Kl+/− mice displayed increased numbers of mtDNA lesions when compared to scramble-treated and wild type controls, respectively (FIG. 4I, J).

α-Klotho Preserves Mitochondrial Function through Maintenance of Mitochondrial Ultrastructure

The disrupted mitochondrial cristae structure within cells displaying decreased α-Klotho expression (FIG. 4A) suggests that α-Klotho is necessary for the maintenance of mitochondrial matrix integrity. Loss of matrix integrity disrupts mitochondrial respiration and induces the accumulation of reactive oxygen species (ROS). ROS accumulation, in turn, drives mtDNA damage and, ultimately, cellular senescence. Thus, it was investigated whether α-Klotho may preserve mitochondrial function through maintenance of mitochondrial ultrastructure. Indeed, TEM analysis reveals a significantly greater number of vacuolated mitochondria with disrupted cristae in the MPCs of Kl+/− mice(FIG. 5A, B).

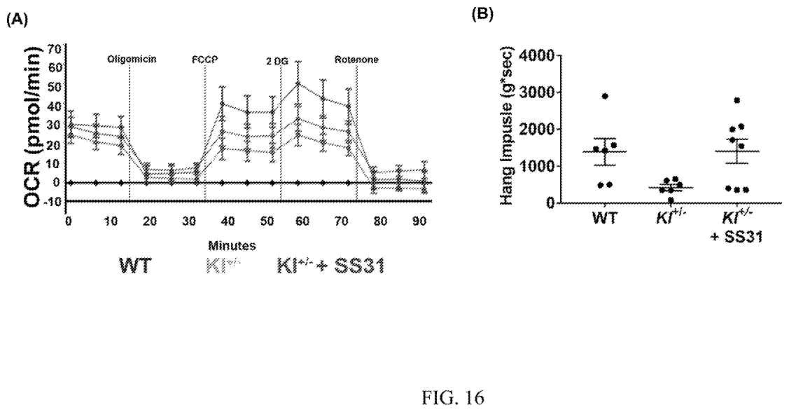

Cardiolipin is an anionic phospholipid that is confined almost exclusively to the inner mitochondrial membrane where it is synthesized. It was found that cardiolipin content is significantly depleted in MPCs isolated from Kl+/− mice, as compared to wild type counterparts (FIG. 5C, D). However, treatment with SS-31, a mitochondrially-targeted peptide that mitigates cardiolipin peroxidation (see, e.g., Szeto et al., Br. J. Pharmacol., 171:2029-50 (2014)), restored Kl+/− MPC ROS generation, mtDNA damage and cellular bioenergetics to levels comparable to wild type counterparts (FIG. 5A-I and FIG. 16). Administration of SS-31 to Kl+/− micealso resulted in an increased number of MyoD+ cells at the site of injury, an enhanced regenerative index, and an increased myofiber cross-sectional area (FIG. 5J-N). Accordingly, Kl+/− mice treated with SS-31 display an increased strength recovery after injury that parallels that of wild type counterparts (FIG. 5O). Of note, treatment of wild type mice with SS-31 did not alter strength recovery after injury when compared to saline-injected counterparts. Taken together, these findings suggest that the defect in MPC mitochondrial bioenergetics and the accumulation of mtDNA lesions resulting from a loss of α-Klotho are mediated by disruption of mitochondrial ultrastructure, the restoration of which results in improved skeletal muscle regeneration.

Supplementation with α-Klotho Restores MPC Bioenergetic Profile In Vitro and Enhances Muscle Regeneration In Vivo

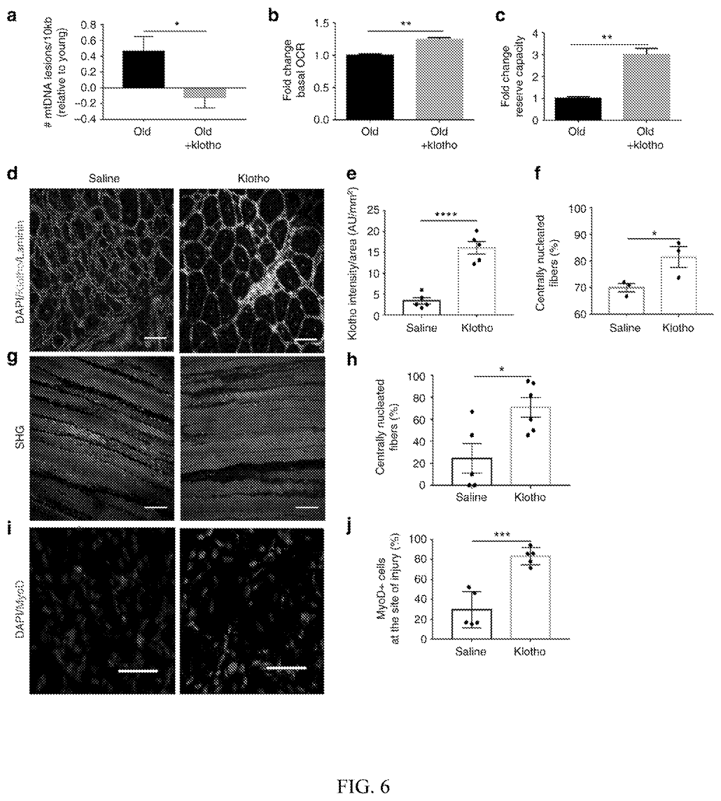

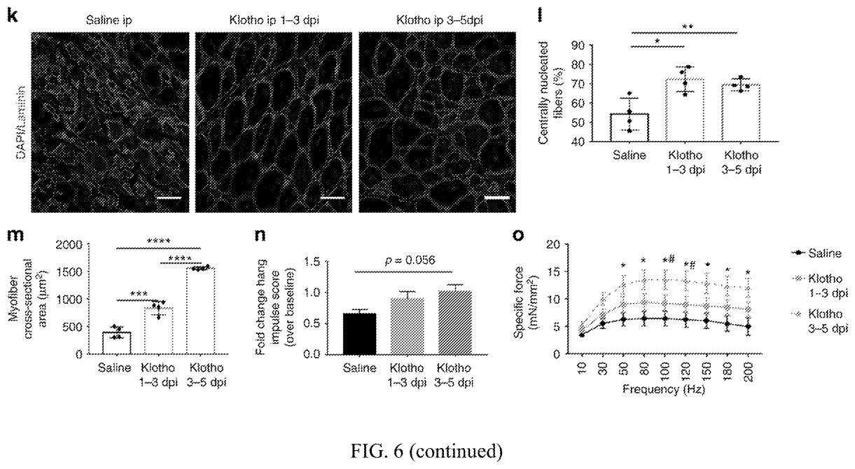

Given the established hormonal role of α-Klotho, it was next investigated whether supplementation of α-Klotho may restore mitochondrial function in aged MPCs. It was found that when MPCs isolated from aged skeletal muscle were cultured in the presence of recombinant α-Klotho for 48 hours, the aged mitochondrial phenotype was improved, as determined by decreased mtDNA damage, an increased OCR, and increased reserve capacity (FIG. 6A-C).

These encouraging in vitro findings led us to next probe whether α-Klotho supplementation may enhance skeletal muscle regeneration in vivo. To do this, α-Klotho was administered to aged mice via osmotic pump delivery. Osmotic pumps were implanted three days prior to injury and were maintained for 14 days post-injury. At the dose tested, a significant increase in local α-Klotho was observed within the injured muscle areas (FIG. 6D, E). Systemic administration of α-Klotho in aged muscle resulted in an increased number of regenerating fibers after injury, as determined both by histology and SHG imaging (FIG. 6D, F-H). These findings were consistent with an approximately 3.5-fold increase in the number of MyoD+ cells at the site of injury in animals that received supplementation with α-Klotho (FIG. 61, J). However, osmotic pump delivery of α-Klotho yielded no significant increase in myofiber cross sectional area or total muscle area, when compared to saline counterparts.

Given that osmotic pump administration delivers α-Klotho continuously, it was next tested whether the timing of α-Klotho administration may be critical for functional tissue regeneration. To do this, daily intraperitoneal injections of recombinant α-Klotho to aged mice either from 1-3 days post injury (dpi) or from 3-5 dpi (i.e. the time point at which we found Klotho to be highly expressed in young muscle) were performed (FIG. 1G). Similar to the findings using osmotic pump administration, α-Klotho delivery both from 1-3 dpi and 3-5 dpi resulted in a significant increase in the number of regenerating fibers when compared to vehicle controls (FIG. 6K, L). Both treatment groups also displayed a significant increase in myofiber area, though the magnitude of the improvement was greater in mice receiving α-Klotho over 3-5 dpi (FIG. 6M). Importantly, only animals receiving α-Klotho over 3-5 dpi displayed an improved functional recovery as determined by the wire hang test and in situ contractile testing (FIG. 6N, O).

Taken together, the data suggest that α-Klotho is required for an adequate regenerative response to an acute injury, and that supplementation with α-Klotho via the circulatory system promotes MuSC commitment and myofiber regeneration in aged mice when administered at the appropriate time point. These findings implicate declines in this longevity protein as a contributor to a defective muscle regenerative response with aging and raise the possibility of systemic administration of α-Klotho as a therapeutic approach to promote the healing of aged skeletal muscle after injury.

### Methods

### Animals

C57BL/6 young (4-6 months) and old (22-24 months) mice were received from the Jackson laboratories and NIA rodent colony, respectively. Kl+/− mice were obtained from MMRRC, UC Davis and were genotyped prior to inclusion in the studies. All animals were ear-tagged, randomly assigned to intervention group, and compared to age-matched littermate controls whenever possible. Mice were evaluated prior to inclusion in the study, and animals with obvious health problems were eliminated. Animal experiments were repeated across a minimum of two separate cohorts of the experimental groups. All primary endpoints were prospectively selected prior to analyses and investigators performing endpoint analysis were blinded to the experimental group whenever possible.

### Animal Injury Model and Histological Analysis of Muscle Regeneration

Wild-type male C57BL/6 young, Kl+/− mice, or old mice received injuries to bilateral Tibialis Anterior (TA) muscles via an intramuscular injection of cardiotoxin (10 μL of 1 mg/mL). Fourteen days following the injury, TAs were harvested for histological analysis of α-Klotho, fibrosis (picrosirius red), degenerating myofibers (IgG) and myofiber regeneration (laminin). Second Harmonic Generation (SHG) imaging was performed on isolated TA muscles treated with a non-targeting control or lentiviral knockdown of α-Klotho, as well as pump-administered animals in order to visualize collagen and myofibers within the muscle 14 days post injury, as described elsewhere (see, e.g., Zhang et al., 2016 Stem Cells 34:732-742).

### Primary Muscle Cell Isolation

MPCs were isolated from young, Kl+−, and aged mice, as described elsewhere (see, e.g., Zhang et al., 2016 Stem Cells 34:732-742). MuSCs were sorted using FACS for surface markers CD31−, CD45−, Sca1− and VCAM+ as described elsewhere (see, e.g., Cheung et al., 2012 Nature, 482:524-8). A modified protocol was used to isolate MuSCs and FAPs as CD31−, CD45−, α-7 integrin+ for MuSCs and CD31−, CD45− and α-7 integrin- for FAPs as described elsewhere (see, e.g., Yi and Rossi, 2011 J Vis Exp. 16:2476).

### Primary Muscle Cell Imaging

Immunofluorescence staining (α-Klotho, Tom20 (mitochondrial marker), ki67, MyoD, Pax7 and HMGB1) and senescence-associated beta-galactosidase staining was performed in isolated cells. Transmission electron microscopy of fixed cells was performed, as described elsewhere (see, e.g., Zhang et al., 2016 Stem Cells 34:732-742). Structured illumination microscopy was performed in young and old cells stained for α-Klotho and DAPI.

### ELISA

The levels of α-Klotho protein were measured by a colorimetric sandwich enzyme immunoassay (SEH757Mu, Cloud-Clone Corp), according to manufacturer's instructions. Each sample was measured in duplicate.

### Hanging-Wire Test

Strength endurance was tested using the hang-wire test as described elsewhere (see, e.g., Aartsma-Rus et al., J. Vis. Exp 85:51303 (2014)). The Hang Impulse (HI) score was calculated as bodyweight (grams)×time hung (seconds). Male mice were used for all testing using C57B1/6 mice. Wild type and Kl+/− were females for testing.

Inhibition of and Supplementation with α-Klotho

MPCs were treated with 25 nmol of silencing RNA (siRNA) to α-Klotho (GE Dharmacon, Product no.SO2462181G) for 48 hours. As a control, young MPCs were treated with a non-targeting (scramble) siRNA. Aged MPCs were treated with 0.05μg/mL exogenous α-Klotho (R & D systems, Product no. aa 34-981), added to the culture media for 48 hours.

### Epigenetic Regulation of α-Klotho

At baseline, three, seven, and fourteen days after injury, TAs were snap frozen using liquid nitrogen for gene expression, methylation specific PCR (MSPCR), and chromatin immunoprecipitation (ChIP) analysis, essentially as described elsewhere (see, e.g., Lin et al., 2016 Am J Respir Cell Mol Biol, 54:241-9).

### Analysis of MPC Bioenergetics and Mitochondrial DNA Damage

Oxygen consumption rate (OCR) and extracellular acidification rate (ECAR) were measured in real time using a Seahorse XFe96 Extracellular Flux Analyzer (Billerica, Mass.) as described elsewhere (see, e.g., de Moura and Van Houten, 2014 Methods Mol Biol, 1105:589-602). The basal OCR was measured by averaging the OCR values before treating the cells with oligomycin. Total reserve capacity was calculated by the differences of OCR between treatment with FCCP and 2DG and basal values. Mitochondrial DNA damage was quantified as described elsewhere (see, e.g., Sanders et al., 2014 Toxicol Sci, 142:395-402; and Sanders et al., 2014 Neurobiol Dis, 62:381-6).

### SS-31 Administration

Isotonic saline or SS-31 (3 mg/kg dissolved in saline at 0.3 mg/mL) was administered daily via an i.p. injection to wild-type and Kl+/− animals for the entire duration of injury. For in vitro experiments, 100 nM of SS-31 was administered to MPCs isolated from Kl+/− animals for 48 hours. Dosing was based on studies demonstrating the effectiveness in a mouse model of chronic cardiomyopathy (see, e.g., Dai et al., 2011 Am Coll Cardiol, 58:73-82) as well as in vitro dose ranging studies performed in C2C12s to evaluate inhibition of stress-induced mitochondrial membrane hyperpolarization and ROS generation.

### In Vivo Lentiviral Knockdown of α-Klotho

In-vivo α-Klotho knockdown was done using lentiviral vectors for a SMARTpool of 2.0×10⁵ TU/TA or 3.82×10⁶ TU/TA shRNA to α-Klotho per TA muscle. Given that there was no significant difference in the local α-Klotho expression between the two treatment groups, samples across the two treatment groups were pooled for analysis. Control animals received equal volumes of empty lentiviral vector. Knockdown was maintained for three weeks, after which time bilateral TAs were injured. Histology or SHG imaging was performed 14 days after injury.

### Supplementation of α-Klotho In Vivo

Mini osmotic pumps containing either saline or α-Klotho (324 pg/ml in saline vehicle) were inserted subcutaneously into aged mice. After 2 days, bilateral TA muscles were injured by intramuscular CTX injection (as above). Osmotic pumps remained implanted until euthanasia 14 days post injury. Isotonic saline or α-Klotho (10 μg/kg body weight) was administered to aged animals via daily intraperitoneal injections over days 1-3 post-injury or 3-5 days post-injury. The TAs were then harvested 14 days post injury and preserved for histology or SHG analysis. Blood serum was also collected to evaluate circulating α-Klotho levels via ELISA. The activity of α-Klotho was confirmed as described elsewhere (see, e.g., Shalhoub et al., 2011 Calcif Tissue Int, 89:140-50).

### Example 2: Exosome-Mediated Delivery of Klotho to Improve Muscle and Brain Function

Isolation of Exosomes from Brain, Plasma and CSF

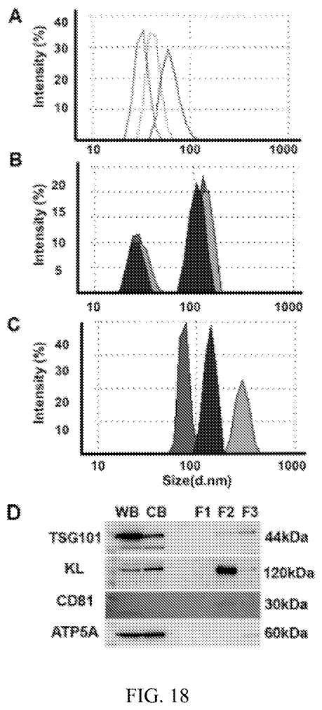

Exosomes were isolated from the brain tissue using a method described elsewhere (see, e.g., Vella et al., 2017 J Extracell Vesicles. 6:1348885). Brain slices from young WT mice (0.5-1 g) were enzymatically digested (collagenase type III in Hybernate E). Exosomes were isolated from CSF (15 μl sample) and plasma (250 μl sample) using a method described elsewhere (see, e.g., Filant et al., Methods Mol Biol. 1740:43-57). Exosomes from these three compartments were isolated using density gradient ultracentrifugation (sucrose) and analyzed using Zetasizer Nano ZS to determine size distribution of each fraction. In the exosome fraction (F2) vesicles detected had average size of 70 nm (FIGS. 18A-C). Other collected fractions were F1 (particles below 20 nm) and F3 (above 300 nm). In the exosome fraction vesicle size was between 30-120 nm. All 3 fractions were immunoblotted for ATP5A (Mitochondrial ATP synthase), which was enriched in non-exosome fractions (see FIG. 18D, WB and CB). This implies that F2 fraction was not contaminated with cellular debris and the used procedure kept cells maximally intact. Enrichment of internal exosome marker TSG101 and tetraspanin CD81, were used to show enrichment of endosome-derived exosomes and separation of particles of the same diameter. Klotho (Kl) is known to be produced in choroid plexus and secreted through exosomes to biofluids. FIG. 18D shows strong enrichment in F2, which clearly demonstrates successful enrichment of exosome proteins in F2.

### Exosomal Delivery of Klotho

Klotho mRNA is Detected in Exosomes Isolated from Human Serum; Expression is Generally Decreased in Individuals with Coronary Heart Disease

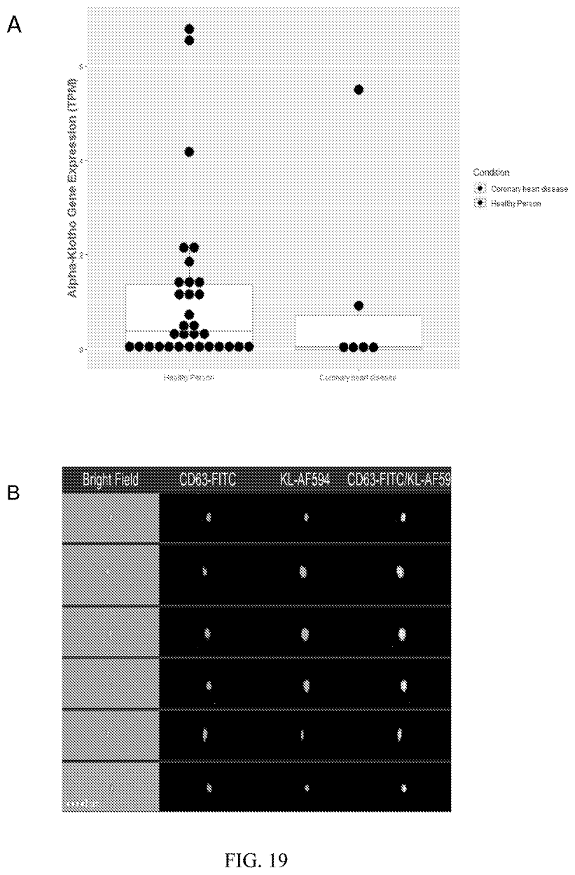

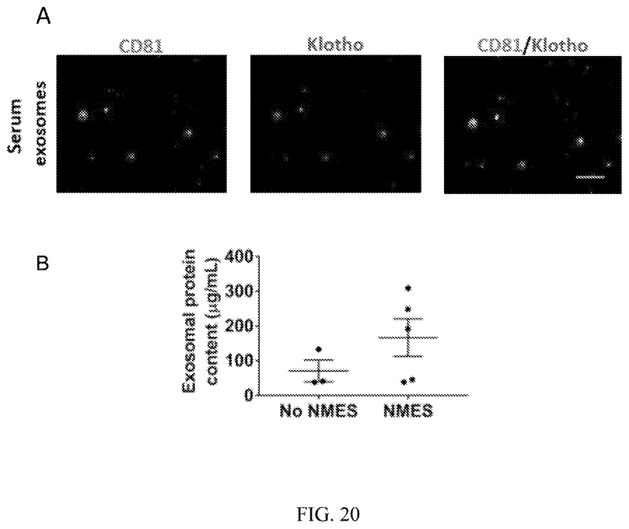

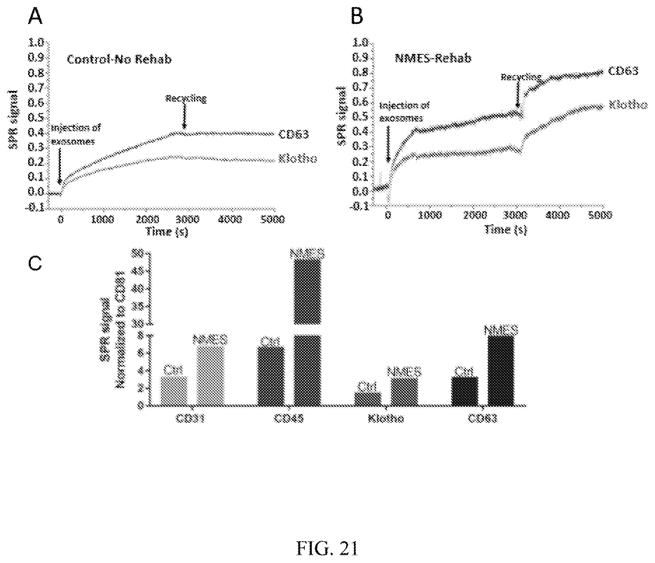

RNAseq data which is housed in a public repository of experimentally validated RNA-seq data analyses from the published literature of human blood exosomes were accessed. Analysis of archived data revealed that Klotho mRNA was detectable in exosomes isolated from human sera (FIG. 19A). However, individuals with coronary heart disease displayed generally lower levels of Klotho mRNA within exosomes (FIG. 19A).

Klotho Protein is Detected in Exosomes Isolated from Brain, Cerebrospinal Fluid (CSF) and Mouse Serum.

Brain slices from young wild type mice (0.5-1 g) were enzymatically digested using collagenase type III in Hybernate E. To isolate exosomes from CSF (15 μL sample) and for plasma (250 μL sample), a method described elsewhere was used (Filant et al., Protocol Exchange (2017)). Exosomes were isolated using density gradient ultracentrifugation (sucrose) and analyzed using Zetasizer Nano ZS to determine size distribution of each fraction. In the exosome fraction (F2), the average size of the vesicles was 70 nm (FIG. 18). Other collected fractions were F1 (particles below 20 nm) and F3 (above 300 nm). In the exosome fraction, vesicle size between 30-120 nm was expected. All three fractions were immunoblotted for ATP5A (Mitochondrial ATP synthase), which was enriched in non-exosome fractions (see FIG. 18D, WB and CB). This implied that F2 fraction was not contaminated with cellular debris, and the used procedure kept cells maximally intact. Enrichment of internal exosome marker TSG101 and tetraspanin CD81 were used to show enrichment of endosome-derived exosomes and separation of particles of the same diameter. Klotho is known to be produced in choroid plexus and is secreted through exosomes to biofluids. FIG. 18D shows strong enrichment in F2, which clearly demonstrated successful enrichment of exosome proteins in F2. In the approach, fractions with particles above or below exosomes size (F1 and F3) were not considered for further analysis. To further confirm the presence of Klotho in murine serum, exosomes were isolated from young mice using a kit such as a commercially available kit (e.g., ExoQuick). Analysis by multispectral flow imaging revealed the presence of Klotho within the CD63+ exosomes (FIG. 19B). Finally, immunofluorescent imaging of exosomes isolated from murine serum reveal co-localization of Klotho and CD81, a widely accepted exosome marker (FIG. 20A).

### Neuromuscular Electrical Stimulation Increases Klotho Protein Expression in Murine Circulating Exosomes.

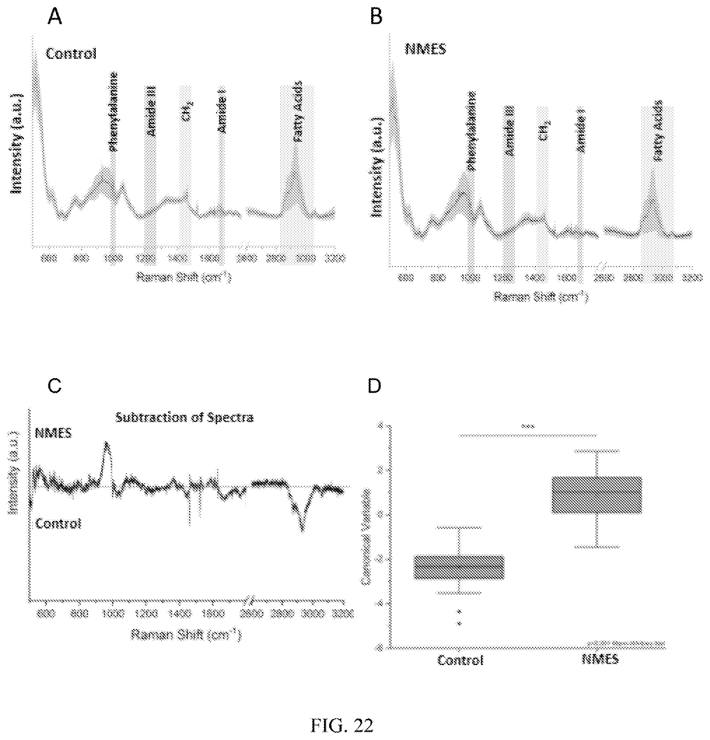

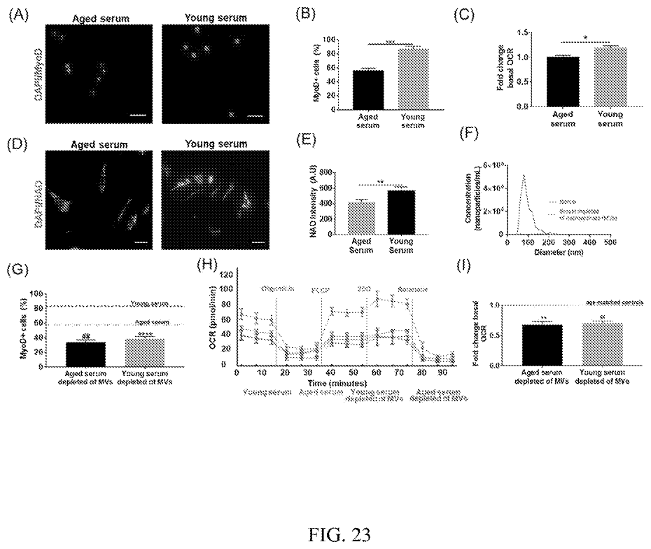

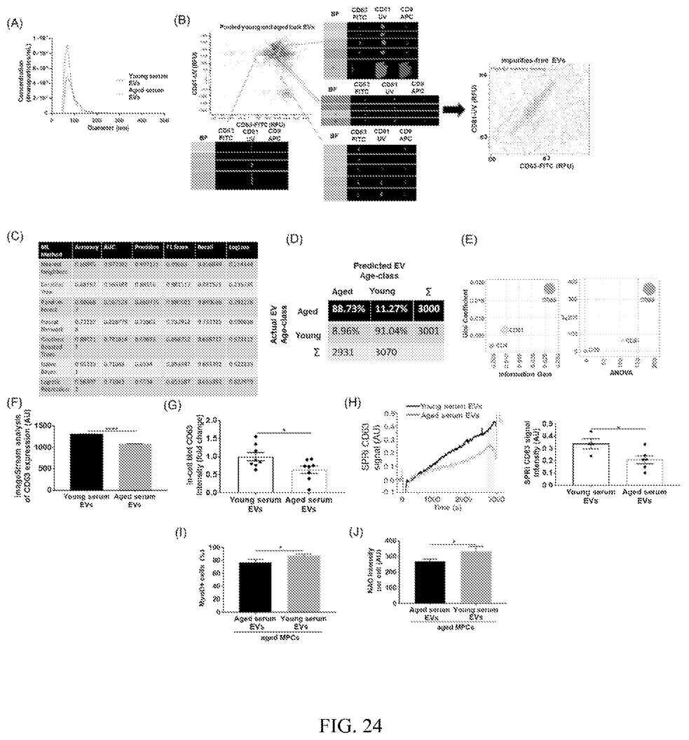

Next, young (4-6 months) mice were exposed to a neuromuscular electrical stimulation (NMES) protocol as described elsewhere (Ambrosio et al., J. Vis. Exp., e3914 (2012)). Exosomes were then isolated using size-exclusion chromatography from NMES stimulated as well as age- and sex-matched sedentary control mice. 2 weeks of NMES increased the amount of protein content within circulating exosomes, as determined by immunofluorescence (FIG. 20B). Raman spectroscopy (FIG. 22A-D) and Surface Plasmon Resonance (FIG. 21A-C) further confirmed an increased Klotho expression in exosomes isolated from serum of aged mice receiving a 2-week NMES protocol.

### Example 3: Extracellular Vesicle Delivery of Klotho Transcripts Rejuvenates Aged Stem Cell Progeny

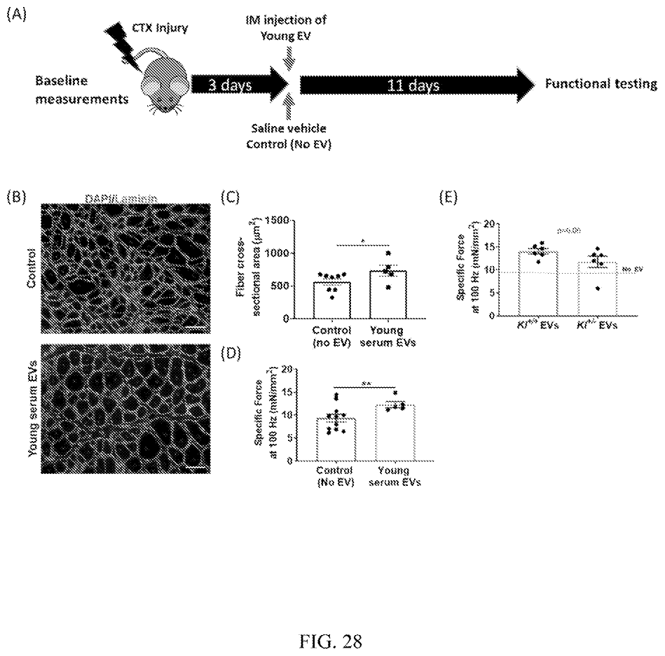

This Example shows that depletion of EVs eliminated the beneficial effect of young serum on the bioenergetics of target MuSC progeny, and that the impact of EVs on target cell mitochondrial function was a result of Klotho mRNA transfer. Machine learning classifiers further revealed that aging disrupts EV population heterogeneity through a selective loss of CD63⁺ extracellular vesicles, which preferentially contain Klotho mRNAs. In vivo, it was shown that Klotho mRNA content within EVs supported muscle regeneration after acute injury (FIG. 28). While transplantation of young EVs enhanced the functional recovery of aged muscle, this benefit was lost when young EVs were derived from Klotho^{+/-} mice (FIG. 28E). Using this gain- or loss-of-function approach, it was demonstrated that Klotho transcripts within CD63⁺ EVs mediated intercellular communications involved in the regenerative cascade.

### Results

### Circulating Extracellular Vesicles Modulate the Bioenergetics of Target Cells in an Age-Dependent Manner

It was found that expression of MyoD, a master regulator of myogenesis, was increased in aged MuSC progeny when cultured in the presence of young serum (FIGS. 23A and 23B). The impact of young versus aged serum on mitochondrial respiration was also evaluated. Aged muscle progenitor cells cultured in the presence of young serum displayed a significantly increased basal oxygen consumption rate when compared to cells exposed to aged serum (FIG. 23C). Young serum also improved mitochondrial integrity of aged progenitor cells, as demonstrated by a 1.5-fold increase in the mitochondrial inner membrane phospholipid, cardiolipin (FIGS. 23D and 23E). These findings demonstrate that serum-derived components preserve target cell mitochondrial ultrastructure and bioenergetics in an age-dependent manner.

To determine whether EVs contribute to the observed effects of young serum on muscle progenitor cell mitochondrial function, young and aged serum were depleted of circulating EVs (FIG. 23F). Immunocytochemical analysis of MyoD and live cell assessment of respiration revealed that the beneficial effect of young serum on target cell MyoD expression and bioenergetics was lost in the absence of circulating EVs (FIGS. 23G-I).

### Aging Shifts Circulating EV Population Heterogeneity Through Preferential Loss of CD63⁺ Extracellular Vesicles

The above findings demonstrate that circulating EVs may regulate the bioenergetics of target muscle progenitor cells, but that aging disrupts this information flow. The transfer of information between EVs and their target cells is mediated by a wide range of EV cargoes, including cytosolic proteins, membrane proteins, mRNAs, noncoding RNAs, and DNA. However, age-associated changes in the circulating EV structure and cargo have not yet been thoroughly investigated. Therefore, an in-depth analysis of young and aged EVs that were isolated using size-exclusion chromatography (qEVsingle, iZON columns) was performed. Nanoparticle tracking analysis using NanoSight apparatus (Product# NS300) confirmed that the size of isolated EVs was less than 200 nm in diameter (FIG. 24A). Interestingly, aged serum yielded a significantly higher concentration of nanoparticles when compared to young serum (FIG. 24A).

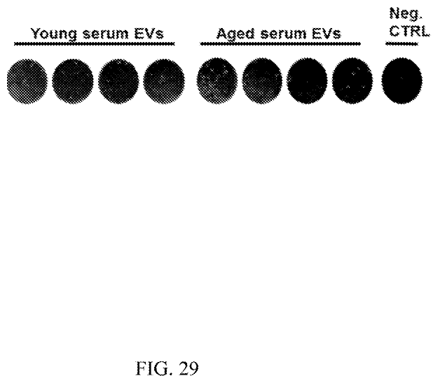

NanoSight quantifies the total concentration of nanoparticles in a non-discriminant manner to include microvesicles, exosomes, and apoptotic bodies. Multispectral flow cytometry imaging (IFC; ImageStream) was used to classify nanoparticles according to differential expression of the three EV markers: CD63, CD81, and CD9. In order to phenotypically resolve the EV signal using IFC, the Fischer's discriminant ratio was utilized as a class separability criterion, followed by feature selection and gating according to intensity-based clustering (FIG. 24B). Machine learning classifiers were then employed to determine whether EV surface markers could be used to predict age-class. Of all classifiers tested, Random Forest classifier had the highest predictive accuracy (˜70%) because of its robustness against mis-labeling and noise (Table 1). These data suggest that aging creates a notable shift in the membrane composition of circulating EVs. To test whether the accuracy of the model could be enhanced “in silico”, the bootstrap method was used, which is a statistical technique that iteratively resamples the dataset to randomly increase the number of observations (FIGS. 24C-D). This approach yielded a classification accuracy of almost 90%, suggesting that the predictive power of EV age-class is tied to population heterogeneity, which is best captured with larger samples. Next, in order to determine which surface EV marker ranked highest in the ability to distinguish between young and aged EVs, information- and statistically-based scoring methods were used, namely, information gain vs. Gini coefficient and ANOVA vs. Chi2As, respectively. Regardless of the method used, CD63 emerged as the most age-discriminative marker (FIG. 24E). Western blotting and Surface Plasmon Resonance imaging (SPRi) confirmed findings of an age-related decline in CD63 (FIGS. 24F-H, FIG. 29).

TABLE 1

| ML Method | Accuracy | AUC | Precision | F1 Score | Recall | LogLoss |
|---|---|---|---|---|---|---|
| Nearest Neighbors | 0.89885 | 0.972382 | 0.897171 | 0.89683 | 0.896849 | 0.214144 |
| Decision Tree | 0.88152 | 0.965188 | 0.88156 | 0.881517 | 0.881521 | 0.235735 |
| Random Forest | 0.880687 | 0.967128 | 0.880735 | 0.880683 | 0.880688 | 0.292276 |
| Neural Network | 0.733378 | 0.826779 | 0.73501 | 0.732918 | 0.733385 | 0.500638 |
| Gradient Boosted Trees | 0.696717 | 0.781014 | 0.69673 | 0.696712 | 0.696717 | 0.573117 |
| Naive Bayes | 0.653391 | 0.71043 | 0.6534 | 0.653387 | 0.653392 | 0.622839 |
| Logistic Regression | 0.563073 | 0.71043 | 0.6534 | 0.653387 | 0.653392 | 0.622839 |

Metrics used to evaluate classifier performance without bootstrapping of data. The training set (⅔ of the entire EV population) contained the known age-class output. The computational model learned on this dataset in order to be generalized to the test dataset.

To address the physiological relevance of age-related changes in EV composition, the direct effect of young or aged EVs on target muscle progenitor cell responses was evaluated. Consistent with serum co-culture experiments described above, it was found that aged cells cultured in the presence of young EVs, but not old EVs, displayed increased MyoD expression and mitochondrial cardiolipin content (FIGS. 24I and 24J). These findings demonstrate that EVs and/or EV cargoes are internalized by recipient progenitor cells to modulate cellular responses, and that functional communication is disrupted with aging.

EV Nucleic Acid Content is Compromised with Aging

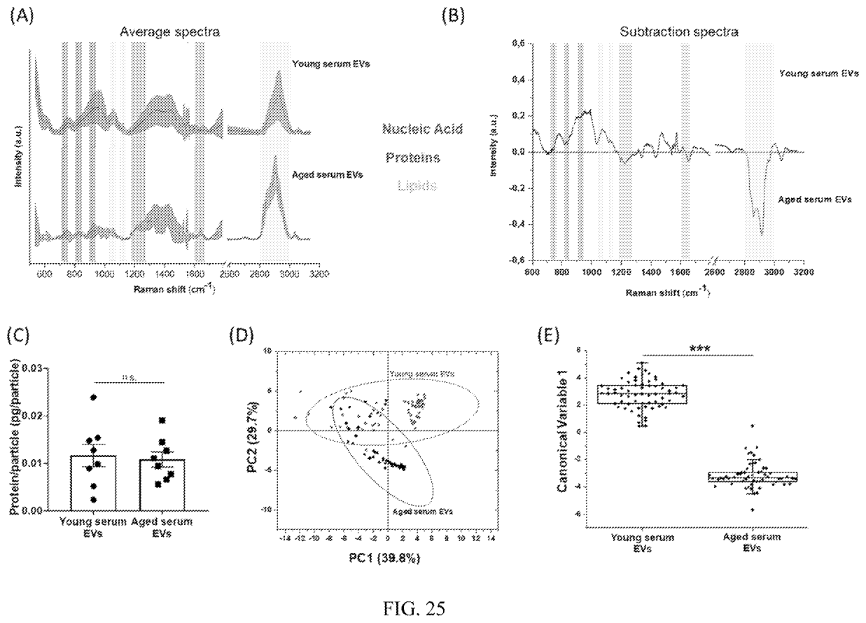

To better understand whether age-related alterations in EV cargo underlie alterations in target cell responses, Raman spectroscopy analysis of young and aged EVs was performed. This method allows for bulk characterization of EV biochemical composition according to light scattering properties. Qualitative EV differences as a function of age were evaluated by subtracting the aged EV spectra from the young, and the resulting Raman spectral peaks were assigned to functional chemical groups, as described elsewhere (see, e.g., Movasaghi et al., Applied Spectroscopy Reviews 42 (2007)). There were no appreciable differences in the protein content of EVs according to age, which was further confirmed by a bicinchoninic acid assay (FIGS. 25A-C). However, aged EVs displayed a marked decrease (Δ Intensity>0.2) in the Raman shift bands that were attributable to nucleic acids and a concomitant increase in lipid content. These findings demonstrate that aging drives remodeling of EV genetic cargo and membrane composition, respectively. A hybrid model of unsupervised (Principal Component Analysis) and supervised (Linear Discriminant Analysis) dimension reduction techniques were then used to graphically express the differential variance of EVs according to age (FIGS. 25D and 25E). Taken together, the label-free and non-invasive Raman fingerprints revealed a distinct compositional biochemical profile of aged EV cargoes.

Klotho Transcripts are Abundant in Young EVs, but Their Content is Decreased with Age

It was next sought to identify specific alterations in the EV cargo that may contribute to changes in target cell responses. The data presented thus far suggest that aging disrupts EV composition and the transfer of mitochondrially-targeted information to recipient MuSC progeny. It was examined whether the transfer of EV cargo alters Klotho protein within recipient cells, thereby regulating mitochondrial function.

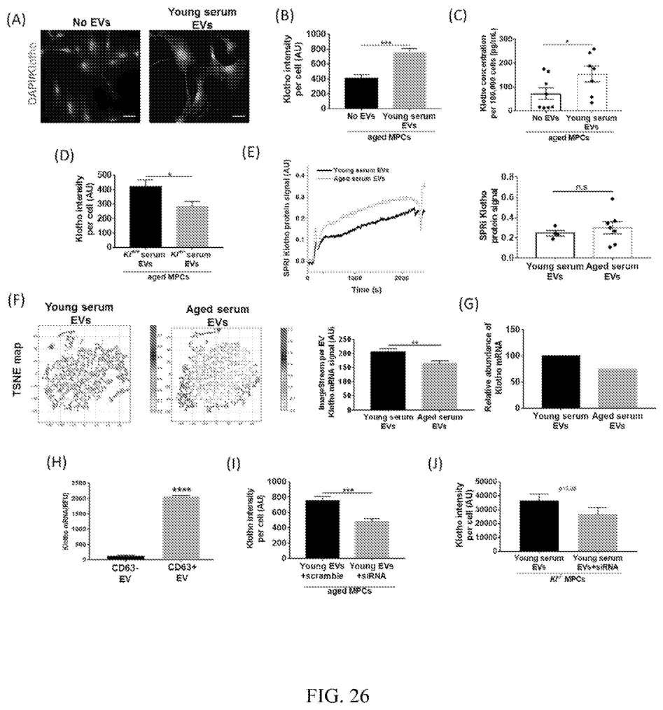

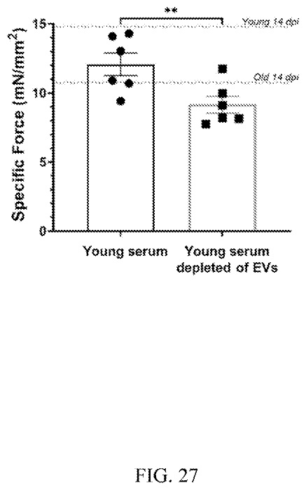

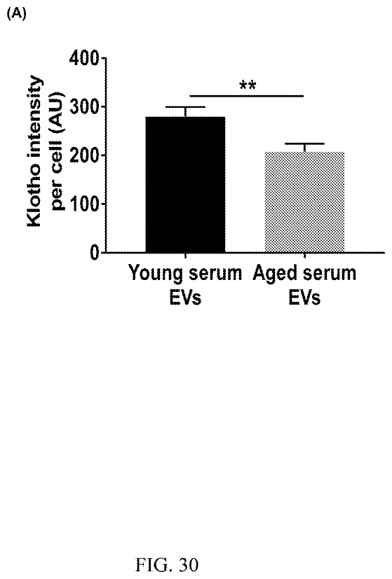

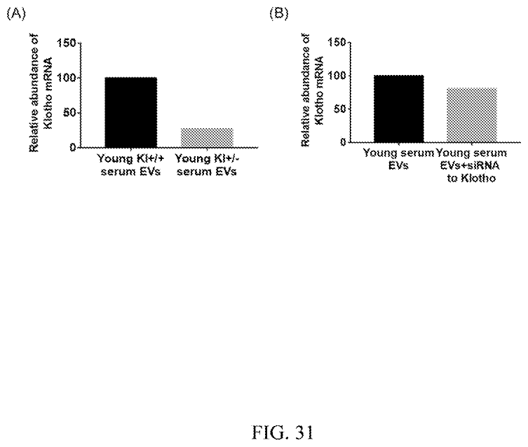

To test this, aged muscle progenitor cells were cultured either in the presence or absence of young serum-derived EVs. Immunofluorescence imaging revealed that Klotho protein in target cells increased by ˜40% when cells were exposed to young EVs (FIGS. 26A and 26B). ELISA analysis of conditioned media further demonstrated that cells cultured in the presence of young EVs displayed increased Klotho expression and secretion (FIG. 26C), suggesting that EVs also promoted Klotho secretion by recipient cells. Cells cultured with aged EVs, however, displayed significantly decreased Klotho protein levels when compared to cells cultured with young EVs (FIG. 30).

To more directly implicate Klotho signals originating from the EVs, the impact of EVs isolated from Klotho^{-/-} mice on Klotho protein levels in target muscle progenitors was tested. It was found that EVs from Klotho^{-/-} mice were toxic to the cells. Cells were then cultured in the presence of EVs isolated from the serum of either Klotho^{+/-} mice or age- and sex-matched wild type control mice. The increased Klotho protein levels observed when aged cells were incubated with young Klotho^{+/+} EVs was blunted when the EVs were isolated from young Klotho^{+/-} mice (FIG. 26D).

Given these data, it was examined whether EVs carry and transmit the Klotho protein in an age-dependent manner. The presence of Klotho protein within serum-derived EVs appears unknown. Surface Plasmon Resonance imaging (SPRi) analysis, however, revealed that circulating EVs do indeed contain Klotho protein on their membranes (FIG. 26E). However, Klotho protein levels were unaltered with aging (FIG. 26E). These findings suggest that Klotho protein within EVs is not likely to explain the age-related changes in EV function on target cells.

The Raman spectroscopy-based finding that aged EVs display a marked decline in nucleic acid content prompted further analysis into whether the increase in Klotho protein by target muscle progenitors may be a result of the transfer of genetically encoded information by EVs. mRNAs packaged within the EVs are functional and capable of being translated when in the presence of the requisite protein machinery of the target cells. To detect and quantify Klotho mRNA within peripheral EVs, a set of Klotho oligonucleotides was designed to probe for Klotho transcripts. IFC revealed that young EVs contained abundant levels of Klotho mRNAs, but that Klotho mRNA content was significantly decreased in aged EVs (FIG. 26F). These findings were confirmed by digital PCR, which demonstrated a decrease of approximately 25% in the Klotho mRNA content in aged EVs (FIG. 26G). EVs isolated from serum of Kl^{+/-} mice expressed about 70% less Klotho mRNA content compared to wild type controls (FIG. 3IA). Intriguingly, it was found that Klotho mRNAs were preferentially carried within CD63⁺ EVs (FIG. 26H), which were found to be decreased in aged serum (FIGS. 24E-H).

To more directly test whether EV-derived Klotho mRNA is a source of Klotho protein within target cells, whether treatment of young EVs with a small interfering RNA to Klotho would abate the Klotho response of recipient progenitors was evaluated. Digital PCR confirmed a decrease in Klotho mRNA of approximately 20% when EVs were treated with siRNA to Klotho (FIG. 3IB). After 48 hours of co-culture, it was found that Klotho protein in aged muscle progenitors exposed to siRNA treated EVs was significantly lower than that from cells cultured in the presence of EVs treated with a scrambled siRNA control (FIG. 26I).

To rule out the possibility that the effect of EVs on progenitor cell Klotho protein expression may result from the transmission of some transcriptional regulator that promotes endogenous Klotho mRNA expression in target cells, young EVs were delivered to muscle progenitor cells isolated from Klotho^{-/-} mice, which lack a functional endogenous Klotho locus. Just as was observed in wild type cells, Klotho^{-/-} cells cultured in the presence of young EVs displayed a significant increase in Klotho protein, and the effect was blunted following treatment with siRNA to Klotho (FIG. 26J). Taken together, these findings demonstrate that circulating EVs increase Klotho protein within target cells via the transfer of Klotho mRNA.

Klotho mRNA Cargoes of Circulating EVs Enhance Muscle Functional Recovery after Injury

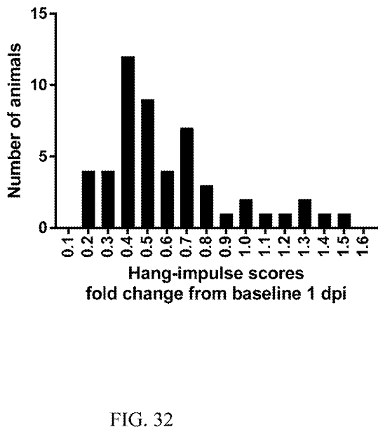

Next, a series of studies was designed to evaluate whether EVs isolated from the circulation may contribute, upon transplantation, to the skeletal muscle regenerative cascade after an acute injury. To induce a muscle injury, aged animals received cardiotoxin injections to bilateral tibialis anterior muscles. The average functional defect one day after injury was approximately 50% of baseline levels (FIG. 32), as determined by hanging grid impulse score. To minimize variability in the extent of injury in experimental groups, only those animals with a functional deficit within the 25-75% percentile one-day post-injury score were randomized to receive either an intramuscular injection of EVs or an equal volume of saline at three days after injury (FIG. 32; FIG. 28A). In situ contractile testing of tibialis anterior (TA) muscles two weeks after injury revealed an increase in peak tetanic force in muscles that received EV transplantation (FIGS. 28B-D). Histological analysis revealed that treatment with EVs also resulted in an increase in the cross-sectional area of regenerating fibers (FIGS. 28B-D), consistent with the enhanced force generation. To directly test whether these enhanced regenerative features were due to the presence of Klotho transcripts in the EVs, EVs isolated from young Klotho^{+/-} animals were injected into injured muscles. Results revealed that the transplantation of Klotho^{+/-} EVs failed to enhance the functional recovery of aged muscle (FIG. 28E).

### Methods

### Serum and Muscle Progenitor Cell Isolation

Serum of young and aged C57/BL6 mice (obtained from Jackson laboratories and NIA Rodent colony, respectively) as well as Klotho^{+/+}, Klotho^{+/-} and Klotho^{-/-} mice (original breeders obtained from MMRCC, UC Davis) was obtained from animals using a cardiac puncture. Skeletal muscle progenitor cells were isolated from aged C57/BL6 (22-24 months) and Klotho^{-/-} male mice (8 weeks) as described elsewhere (see, e.g., Sahu et al., Nat. Commun., 9:4859 (2018)).

### Immunofluorescence Imaging

Immunofluorescence staining for Klotho (R&D systems, MAB1819) and MyoD (SCBT, sc-760) as well as nonyl acridine orange (NAO) staining (Thermofisher, A1372) for cardolipin content was performed on aged cells across experimental groups. Muscle sections were analyzed for fiber cross-sectional area using an antibody against Laminin (Abcam, ab11575). Imaging was performed at 20× magnification on Zeiss-Axiovision microscope.

### Analysis of Cellular Bioenergetics

Oxygen consumption rate (OCR) was measured in real time using a Seahorse XFe96 Extracellular Flux Analyzer (Billerica, Mass.) as described elsewhere (see, e.g., de Moura et al., Methods Mol. Biol., 1105:589-602 (2014)).

### EV Isolation and Characterization

EVs were isolated from serum of young, aged, Klotho^{+/+} and Klotho^{+/-} animals using size-exclusion chromatography (qEVsingle-35 nm iZON columns) according to manufacturer's instructions. The EVs were characterized for size by Nanoparticle Tracking Analysis on NanoSight NS300. EVs were then characterized for CD63 (SCBT 5275), CD81 (SCBT 23962), and CD9 (SCBT 13118) markers using multispectral flow cytometry based ImageStream analysis. EV marker CD63 was further confirmed using SPRi and in-cell western blot.

### ImageStream Imaging and PrimeFlow™ RNA Assay

EVs isolated from young and aged serum were analyzed using Amnis® ImageStream®XMark II (Luminex Corporation). First, samples were processed with filtered sheath buffers to ensure the removal of big particulates and debris (≥1 μm). Flow cytometry was then performed using a 60× objective at a resolution of 0.3 μm²/pixel. Both brightfield and fluorescent images of the EVs were captured using the INSPIRE® software with the highest resolution (sensitivity) and lowest speed. An integrative technical computing framework with multiple machine learning modules and statistical analyses was utilized using R/Python and Wolfram programming languages to analyze the EV signals.

PrimeFlow™ was performed according to the manufacturer's instructions. Two standard 20bDNAs Mouse Klotho oligos probe sets (VB1-6001084 (Part No. 6003837) and VB10-6001085 (Part No. 6003838)) tagged with Type 1 Alexafluor (AF)647 and Type 10 Alexafluor (AF) AF568 dyes, respectively were utilized. mRNA expression was reported based on the mean fluorescence intensity (MFI) at the single EV resolution.

### SPR Imaging and Analysis

EVs were injected into the flow cell of the SPRi instrument XelPleX (Horiba Scientific SAS). The EVs were then injected over a gold chip (SPRi-Biochip, Palaiseau, France) onto which antibodies against CD63 and Klotho were spotted using a micro-spotter (SPRi Arrayer, Horiba). EzSuite software and OriginLab software were used to analyze the collected sensograms.

Raman Spectroscopy Young and aged EVs isolated by size-exclusion chromatography were concentrated by an ultracentrifugation step (100,000 g×70 minutes). These EVs were then analyzed by means of Raman spectroscopy (LabRAM, Horiba Jobin Yvon S. A. S. Lille, France) as described elsewhere (see, e.g., Gualerzi et al., Sci. Rep., 7:9820 (2017)).

### ELISA

Aged muscle progenitor cells were cultured at 10,000 cells per well of an 8-well chamber slide, for 24 hours prior to treatment with young EVs. The conditioned media was collected 48 hours post-administration, and levels of Klotho protein in conditioned media were measured by a colorimetric sandwich enzyme immunoassay (SEH757Mu, Cloud-Clone Corp), according to manufacturer's instructions. The protein concentration was then normalized to total number of cells per well.

### Functional and Histological Analysis of Muscle Regeneration of Injured Animals

Wild-type male C57BL/6 (22-24 months) and Klotho^{+/-} mice (4-7 months) mice received injuries to bilateral Tibialis Anterior (TA) muscles via an intramuscular (i.m.) injection of cardiotoxin (10 μL of 1 mg/mL). Three days post-injury, the animals received 20-30 μL of bilateral i.m. injections of EVs, and in situ contractile testing was performed two weeks after injury as described elsewhere (see, e.g., Zhang et al., Stem Cells 34:732-742 (2016)). The overall muscle endurance of mice was tested at one- and 13-days post injury using a modified hanging-grid test (see, e.g., Sahu et al., Nat. Commun., 9:4859 (2018); and Aartsma-Rus et al., J. Vis. Exp., 85:e51303 (2014)). The hang time for each mouse was normalized to mouse weight. TA muscles were harvested for histological analysis of myofiber regeneration using an antibody against Laminin (Abeam, ab11575). All animals were randomly assigned to intervention group based on their baseline hang-impulse scores and were compared to age-matched littermate controls whenever possible.

### Steps to Ensure Rigor

For all experiments, investigators performing endpoint analyses were blinded to the treatment group. To do this, animals were ear-tagged for in vivo analyses, and samples were number-coded. Animals with obvious health problems were eliminated prior to inclusion in the study. All animals meeting criteria for inclusion were then randomly assigned to treatment groups.

### Code Availability

The computational code used to perform machine learning based analyses on EVs are available online at github.com/SelfHorizonsWork/Nature-Extracellular-vesicle-delivery-of-Klotho-transcripts-rejuvenates-aged-stem-cell-progeny. To generate PCAs, the OriginLab plugin called “Principal Component Analysis for Spectroscopy” was used.

### Example 4: Engineering EVs with Synthetic Klotho mRNA

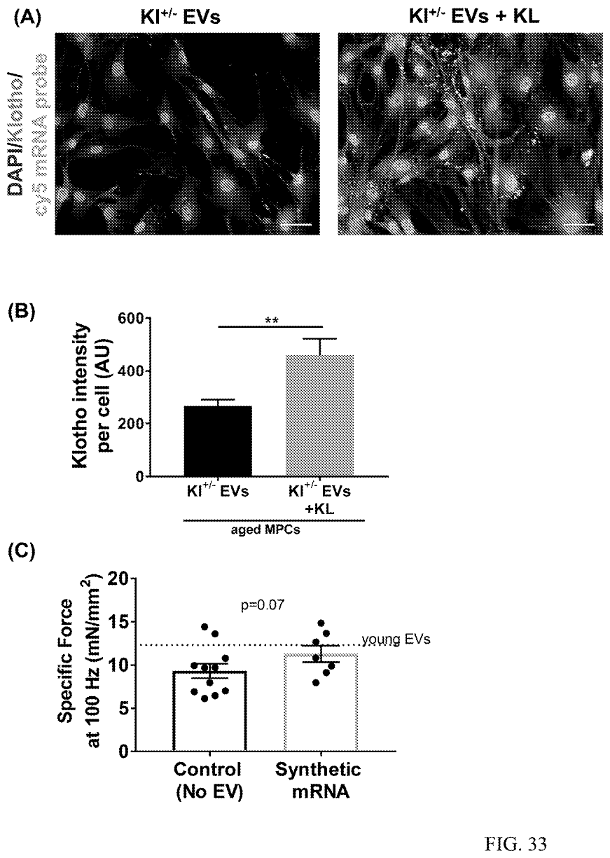

EVs with synthetic Klotho mRNA were engineered. Briefly, EVs were transfected with the synthetic mRNA sequences (Klotho oligos) using Exo-Fect™ Exosome Transfection Reagent from System Biosciences (Cat. No. EXFT-10A1). The transfection was done as per manufacturer's suggested protocol. Briefly, ˜e9 EVs received 150 μL exofect solution and 1 μg amount of synthetic mRNA. This solution was mixed well by flicking the tube 3 times. Samples were incubated at 37° C. for 10 minutes and then put on ice for 30 minutes to stop the reaction. Samples were then centrifuged at 13k-14k rpm. The supernatant was removed, and sample was resuspended in media for in vitro application. 10k aged muscle progenitors were incubated with loaded EVs for 48 hours (FIGS. 33A, B). For in vivo applications, the loaded EVs (˜7.5e8-e9 EVs) were administered to aged animals through intramuscular injections on third and fifth days post injury. In situ contractile testing was performed on the injured TAs 14 dpi (FIG. 33C).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for reducing sarcopenia or age-related cognitive decline within a mammal, wherein said method comprises administering exosomes comprising an α-Klotho polypeptide or a nucleic acid encoding said α-Klotho polypeptide to said mammal.

2. The method of claim 1, wherein said mammal is a human.

3. The method of claim 1, wherein said method comprises administering exosomes comprising said α-Klotho polypeptide to said mammal.

4. The method of claim 1, wherein said method comprises administering exosomes comprising said nucleic acid to said mammal.

5-9. (canceled)

10. A method for increasing the ability of muscle progenitor cells to regenerate muscle cells within a mammal having a muscle impairment, wherein said method comprises:
(a) identifying said mammal as having said muscle impairment, and
(b) administering an α-Klotho polypeptide to said mammal.

11. (canceled)

12. The method of claim 10, wherein said mammal is a human.

13. The method of claim 10, wherein said muscle impairment is sarcopenia.

14-20. (canceled)

21. A method for increasing the ability of muscle progenitor cells to regenerate muscle cells within a mammal, wherein said method comprises administering an exosome comprising an α-Klotho polypeptide or a nucleic acid encoding said α-Klotho polypeptide to said mammal.

22. The method of claim 21, wherein said mammal is a human.

23. The method of claim 21, wherein said mammal has sarcopenia.

24. The method of claim 21, wherein said method comprises administering an exosome comprising said α-Klotho polypeptide to said mammal.

25. The method of claim 21, wherein said method comprises administering an exosome comprising said nucleic acid to said mammal.

26-49. (canceled)
